(19) United States
(12) Patent Application Publication
(10) Pub. No.: US 2005/0214376 A1
(43) Pub. Date: Sep. 29, 2005

(54) Hydrogel-containing medical articles and methods of using and making the same

(76) Inventors: Marie-Pierre Faure, Ville St. Laurent (CA); Marielle Robert, St-Hyacinthe (CA)

Correspondence Address: KIRKPATRICK & LOCKHART NICHOLSON GRAHAM LLP;(FORMERLY KIRKPATRICK & LOCKHART LLP), 75 STATE STREET, BOSTON, MA, 02109-1808, US

(21) Appl. No.: 10/970,349

(22) Filed: Oct. 21, 2004

Related U.S. Application Data
(60) Provisional application No. 60/512,866, filed on Oct. 21, 2003.

(51) Int. Cl. A61K 9/14; A61K 38/16

(52) U.S. Cl. 424/486; 514/12

(57) ABSTRACT

Medical articles including a hydrophilic water-swellable hydrogel and methods of using and making the articles are provided. The hydrogel may include a crosslinked mixture of a biocompatible polymer and a protein, such as polyethylene glycol and a soy protein. The hydrogel may further include an agent, such as diazolidinyl urea and iodopropynyl butylcarbamate, dispersed within the hydrophilic water-swellable hydrogel.

### CROSS-REFERENCE TO RELATED APPLICATIONS

The present application claims priority to and the benefit of commonly-owned U.S. Provisional Application No. 60/512,866, filed on Oct. 21, 2003, the entire disclosure of which is incorporated herein by reference.

### FIELD OF THE INVENTION

This invention relates generally to medical articles comprising a high-water-content hydrogel made by crosslinking a protein with activated polyethylene glycols. The medical articles may further include an active agent, such as an agent that confers antimicrobial, analgesic, and/or wound healing activities to the hydrogel. The invention further provides methods for treating a wound using the medical articles described. Such methods may include delivering an active agent to a wound or to an intact topical site.

### BACKGROUND OF THE INVENTION

Acute, infected and chronic wounds affect millions of patients a year. They significantly impair the quality of life of the affected patients and pose an enormous burden on society in terms of lost productivity and health care costs. Wounds can be caused by a variety of events, including surgery, prolonged bedrest, diseases (e.g., diabetes), and traumatic injuries. Characteristics of chronic wounds include a loss of skin or underlying tissue and the failure to heal with conventional types of treatment. This failure is mostly due to microbial contamination of the wounds.

The wound healing process involves a complex series of biological interactions at the cellular level and is generally considered to occur in several stages, known as the healing cascade. At the inflammatory phase, fibroblast cells are stimulated to produce collagen. During the proliferative phase, reepithelialization occurs as keratinocytes migrate from wound edges to cover the wound, and new blood vessels and collagen are laid down in the wound bed. Finally, at the maturation phase, collagen is remodeled into a more organized structure, eventually resulting in the formation of a scar.

It is commonly accepted that a moist environment helps to promote reepithelialization, which typically leads to faster healing of a wound. Traditional dry wound treatment with, for example, gauze compresses, are thus undesirable for the treatment of wounds although they are still used in hospitals.

Although improper wound treatment can contribute to poor wound healing, the most common cause of resisted wound healing is likely wound infection. Despite the fact that many of the microorganisms commonly found in wounds usually exist as commensals in their natural human habitats, cutaneous wounds of both acute and chronic origin provide an especially favorable environment for microbial growth. In particular, leg ulcers, pressure ulcers, diabetic foot ulcers, and fungating wounds typically harbor diverse and often dense microbial populations involving both aerobic and anaerobic microorganisms. The ability of the immune system to defend a wound infection in these cases is impaired, as trauma and necrosis of the skin decrease vascularization to a wound and the influx of immunologic proteins and white blood cells. The wound healing cascade, in turn, is delayed until the inflammatory and physiologic debridement phases have killed and removed contaminating microbes and necrotic tissues. Severe-burn victims therefore are particularly susceptible to microbial infections due to their compromised immune system, and present an especially challenging case for wound management.

While clinicians frequently focus on the type of microbes that may contaminate a wound, some studies suggest that the number of invading microbes is more important than the species. A microbial count in excess of 100,000 organisms per gram of tissue typically leads to a wound infection. Proliferating microbes cause additional and accelerated tissue damage through both direct (toxins and cellular damage) and indirect (edema and accumulation of pus) impairment of vascular supply. These changes further impair access of immune system components to the wound as well as reducing the clearance of necrotic debris and preventing systemically delivered antibiotics from reaching contaminated tissues. Collagenase and proteases that accumulate in association with degenerating inflammatory cells damage connective tissue proteins and further inhibit wound healing.

Meanwhile, nosocomial infection has long been recognized as one of the leading causes of death in United States. A large percentage of nosocomial infections are device-related. For example, many patients using a long-term in-dwelling urinary catheter will end up contracting urinary tract infections. Whenever an in-dwelling medical device punctuates the skin, the host tissue reacts to the device as a foreign body and deposits a thrombin coat over the material, which becomes colonized with microbes. In this coating of protein and microorganisms, known as the biofilm, microbes find a suitable niche for continued growth as well as for protection from antibiotics, phagocytic neutrophils, macrophages and antibodies. The skin insertion site, therefore, is most often the source of catheter-related sepsis and infection. Accordingly, proper care of the skin insertion site is believed to be the most effective way of preventing and treating nosocomial infection.

While some in-dwelling medical devices claim to have antimicrobial properties—for instance, their entire external surface may be coated with an antimicrobial agent, these devices often do not target the skin insertion site (i.e., the infection site) specifically. Besides, coating or incorporating an antimicrobial agent along the entire external surface of the in-dwelling device is impractical and uneconomic, and the antimicrobial agent may present other side effects when introduced systematically at a high concentration. It is generally accepted that the treatment of biofilm-mediated infection on the surface of medical devices is currently extremely difficult, and that no satisfactory medical device or method has yet emerged to treat in-dwelling medical device-related infections.

Attempts have been made to provide improved wound dressings that are composed partially or entirely of hydrogels. Hydrogels are generally prepared by polymerization of a hydrophilic monomer under conditions where the polymer becomes crosslinked in a three-dimensional matrix sufficient to gel the solution.

U.S. Pat. No. 5,527,271 describes a composite material made from a fibrous material, such as cotton gauze, impregnated with a thermoplastic hydrogel-forming copolymer containing both hydrophilic and hydrophobic segments. While the wound dressings absorb wound exudate which facilitates healing, they are problematic in that fibers of the cotton gauze may adhere to the wound or newly forming tissue, thereby causing wound injury upon removal. In addition, as the hydrogel is impregnated within the fibrous material, the hydrogel can only provide minimal hydrating effect.

U.S. Pat. App. Pub. No. 2004/0142019 describes a wound dressing comprising microbial-derived cellulose in an amorphous gel form. The wound dressing is described as having a flowable nature, which supposedly allows it to fill up the wound bed surface. The lack of a defined structure, however, makes it potentially difficult to manipulate.

### SUMMARY OF THE INVENTION

Thus, there remains a need for a wound dressing that protects the injured tissue, maintains a moist environment, and sufficiently adheres to a wound without causing pain or further injury upon removal. Further, the wound dressing typically should be water-permeable, easy to apply, inexpensive to make, and/or conform to the contours of the skin or other body surface, both during motion and at rest. Additionally, the wound dressing typically should be translucent, thus making it possible to visually inspect a wound without removing the dressing, should not require frequent changes, and/or should be non-toxic and non-allergenic. More importantly, the wound dressing typically should have antimicrobial properties, allowing it to prevent and/or treat microbial infections. It would also be beneficial if the wound dressing can further deliver pharmaceutical agents to the wound site to assist healing.

Furthermore, there remains a need for medical articles that can prevent or treat nosocomial infections, especially those due to catheterization, and for methods for deterring microbial biofilm development on the surface of in-dwelling medical devices in contact with tissue, especially at the skin insertion site.

The present invention provides a medical article which can possess any or all of the advantageous properties listed above, and which is especially suitable to be used as a wound dressing or a drug delivery platform.

In its most general application, the present invention provides a medical article that includes a hydrophilic water-swellable hydrogel having a crosslinked mixture of a biocompatible polymer and a protein. The medical article may further include a pharmaceutical agent dispersed within the hydrogel matrix, to confer a desirable activity to the medical article.

In one aspect, the medical article may include the hydrophilic water-swellable hydrogel described above and at least one of diazolidinyl urea and iodopropynyl butylcarbamate dispersed within the hydrogel. In some embodiments, the biocompatible polymer may include polyethylene glycol. The protein may include albumin, which may be obtained from a vegetal source, such as soybean. In certain embodiments, the medical article may further include a support. The support may include a polymeric surface, to which the hydrophilic water-swellable hydrogel may be attached.

In some embodiments, the medical article may include an in-dwelling member, such as a catheter. The in-dwelling member may include a first portion adapted to be inserted into the body of a patient and a second portion adapted to be exposed outside the body of a patient. The hydrophilic water-swellable hydrogel may be disposed about the in-dwelling member at a point along the second portion of the in-dwelling member. In some embodiments, the hydrogel may include a longitudinal slot or an opening of other shapes with a dimension adapted to allow at least the second portion of the in-dwelling member to pass through. The hydrogel may be disposed on or around an anatomical site of the patient, the anatomical site being the point of insertion of the in-dwelling member.

In another aspect, the present invention provides a method for treating a wound. The method includes administering to a wound the medical article described above such that wound healing occurs faster as compared to a wound being treated in an identical manner by another medical article which includes a polyurethane membrane coated with a layer of an acrylic adhesive. In some embodiments, the rate of wound healing is determined by measuring at least one criterion selected from the group consisting of reduction of wound size, amount of time to achieve wound closure, contrast between wound color and normal tissue color, signs of infection, or duration of the inflammatory phase.

In a third aspect, the present invention provides a method for treating a wound, for example, to prevent infection. The method includes applying to an anatomical site of a mammal the medical article described above. The anatomical site may include a topical site.

In a fourth aspect, the present invention provides a method for treating an infected wound. The method includes applying a medical article to the wound. The medical article may include a hydrating component, which includes a hydrophilic water-swellable hydrogel comprising a crosslinked mixture of a biocompatible polymer and a protein, and an oxidizing agent dispersed within the hydrogel which is in a therapeutically effective amount to generate an antimicrobial effect.

In a fifth aspect, the present invention provides a method for preparing a medical article. The method includes loading a hydrophilic water-swellable hydrogel including a crosslinked mixture of a biocompatible polymer and a protein with a solution including at least one of diazolidinyl urea and iodopropynyl butylcarbamate. In some embodiments, the solution may further include an acid, a base, or a buffer sufficient to adjust the pH of the solution to a range of about 3.0 to about 9.0.

In a sixth aspect, the present invention provides a method for delivering lidocaine to a patient. The method includes apply to at least one region of a patient a medical article including lidocaine and a hydrophilic water-swellable hydrogel including a crosslinked mixture of a biocompatible polymer and a protein from a source selected from a vegetal source or a marine source. The protein may be a soy protein. In some embodiments, the one region of the patient may be epidermis. The epidermis may be physically intact or it may include an open wound.

In a seventh aspect, the present invention provides a method for delivering an agent to a wound. The method includes applying to a wound a medical article including an agent and a hydrophilic water-swellable hydrogel including a crosslinked mixture of a biocompatible polymer and a protein from a source selected from a vegetal source or a marine source. The protein may be a soy protein. The agent may include a therapeutically effective amount of a physiologically active compound to be delivered to the wound. The physiologically active compound may include lidocaine. The agent may include a preservative, such as diazolidinyl urea and iodopropynyl butylcarbamate. The agent may be transportably present in the hydrogel. The hydrogel may further be loaded with a solution having a pH value in the range of about 3.0 to about 9.0.

### BRIEF DESCRIPTION OF THE DRAWINGS

The objects and features of the invention can be better understood with reference to the drawings described below, and the claims. The drawings are not necessarily to scale, emphasis instead being placed upon illustrating the principles of the invention.

FIG. 1 is a schematic illustration of an embodiment of the invention including an in-dwelling member.

FIG. 2 is a graphical representation of the amount of water that can be retained in certain hydrogel embodiments, expressed as a weight percentage relative to the weight of the swollen hydrogel (i.e., the water content), when the hydrogel embodiments are prepared with various protein solutions that have been diluted with a phosphate buffer solution having concentrations between 10 mM and 100 mM.

FIG. 3 is a graphical representation of the correlation between the water uptake value of certain hydrogel embodiments and the concentration of the phosphate buffer solution used to dilute the various protein solutions for preparing the hydrogel embodiments.

FIG. 4 is a graphical representation of the amount of water that can be retained in certain hydrogel embodiments, expressed as a weight percentage relative to the weight of the swollen hydrogel (i.e., the water content), when the hydrogel embodiments are prepared with various protein solutions that have been diluted with a phosphate buffer solution having pH values between 4 and 11.

FIG. 5 is a graphical representation of the correlation between the water uptake value of certain hydrogel embodiments and the pH value of the phosphate buffer solution used to dilute the various protein solutions for preparing the hydrogel embodiments.

FIG. 6 is a graphical representation of the correlation between the expansion volume of certain hydrogel embodiments and the concentration of the phosphate buffer solution used to dilute the various protein solutions for preparing the hydrogel embodiments.

FIG. 7 is a graphical representation of the correlation between the expansion volume of certain hydrogel embodiments and the pH value of the phosphate buffer solution used to dilute the various protein solutions for preparing the hydrogel embodiments.

FIG. 8 shows the relative uptake of p-nitrophenol and methylene blue by certain hydrogel embodiments as a function of time.

FIG. 9A shows the cumulative amount of caffeine that was released from an embodiment of the invention and delivered across the skin barrier over a 24-hour period, the quantity of caffeine being expressed in micrograms, in comparison to caffeine being delivered from a solution as studied in vitro under non-occlusive conditions.

FIG. 9B shows the cumulative amount of caffeine that was released from an embodiment of the invention and delivered across the skin barrier over a 24-hour period, the quantity of caffeine being expressed in micrograms, in comparison to caffeine being delivered from a solution as studied in vitro under occlusive conditions.

FIG. 9C shows the flux of caffeine delivery from a solution and by an embodiment of the invention as measured over a 24-hour period in vitro under non-occlusive conditions.

FIG. 9D shows the flux of caffeine delivery from a solution and by an embodiment of the invention as measured over a 24-hour period in vitro under occlusive conditions.

FIG. 10A shows the water content in certain embodiments of the invention with different concentrations of caffeine as applied to the skin in vitro under non-occlusive conditions.

FIG. 10B shows the water content in certain embodiments of the invention with different concentrations of caffeine as applied to the skin in vitro under occlusive conditions.

FIG. 11A shows the relative variation in skin hydration after a 2-hour application of certain embodiments of the invention on human subjects under non-occlusive conditions.

FIG. 11B shows the relative variation in skin hydration after a 24-hour application of certain embodiments of the invention on human subjects under occlusive conditions.

FIG. 12A shows the permeation profiles of caffeine as released from three different embodiments of the invention (each includes a hydrogel having been loaded with a 0.5%, 1%, and 2% (by weight) caffeine solution, respectively) over a 24-hour period in vitro under non-occlusive conditions.

FIG. 12B shows the permeation profiles of caffeine as released from three different embodiments of the invention (each includes a hydrogel having been loaded with a 0.5%, 1%, and 2% (by weight) caffeine solution, respectively) over a 24-hour period in vitro under occlusive conditions.

FIG. 12C is a graphical representation of the caffeine flux that corresponds to the permeation profiles of FIG. 12A.

FIG. 12D is a graphical representation of the caffeine flux that corresponds to the permeation profiles of FIG. 12B.

FIG. 13A shows the permeation profiles of caffeine as released from six different embodiments of the invention (each includes a hydrogel having been loaded with either a 0.5% or 2% (by weight) caffeine solution and having a pH of 3.0, 5.5, and 9.0, respectively) over a 24-hour period in vitro under non-occlusive conditions.

FIG. 13B shows the permeation profiles of caffeine as released from six different embodiments of the invention (each includes a hydrogel having been loaded with either a 0.5% or 2% (by weight) caffeine solution and having a pH of 3.0, 5.5, and 9.0, respectively) over a 24-hour period in vitro under occlusive conditions.

FIG. 13C is a graphical representation of the caffeine flux that corresponds to the permeation profiles of FIG. 13A.

FIG. 13D is a graphical representation of the caffeine flux that corresponds to the permeation profiles of FIG. 13B.

FIG. 14A shows the permeation profiles of caffeine as released from six different embodiments of the invention (each includes a hydrogel having been loaded with either a 0.5% or 2% (by weight) caffeine solution and having a thickness of 1.45 mm, 2.9 mm, and 4.35 mm, respectively) over a 24-hour period in vitro under non-occlusive conditions.

FIG. 14B shows the permeation profiles of caffeine as released from six different embodiments of the invention (each includes a hydrogel having been loaded with either a 0.5% or 2% (by weight) caffeine solution and having a thickness of 1.45 mm, 2.9 mm, and 4.35 mm, respectively) over a 24-hour period in vitro under occlusive conditions.

FIG. 14C is a graphical representation of the caffeine flux that corresponds to the permeation profiles of FIG. 14A.

FIG. 14D is a graphical representation of the caffeine flux that corresponds to the permeation profiles of FIG. 14B.

FIG. 15A shows the permeation profiles of caffeine as released from six different embodiments of the invention (each includes a hydrogel having been prepared with one of six different types of protein and loaded with a 2% (by weight) caffeine solution) over a 24-hour period in vitro under non-occlusive conditions.

FIG. 15B shows the permeation profiles of caffeine as released from six different embodiments of the invention (each includes a hydrogel having been prepared with one of five different types of protein and loaded with a 2% (by weight) caffeine solution) over a 24-hour period in vitro under occlusive conditions.

FIG. 15C shows the permeation profiles of caffeine as released from six different embodiments of the invention (each includes a hydrogel having been prepared with one of six different types of protein and loaded with a 0.5% (by weight) caffeine solution) over a 24-hour period in vitro under non-occlusive conditions.

FIG. 15D shows the permeation profiles of caffeine as released from six different embodiments of the invention (each includes a hydrogel having been prepared with one of five different types of protein and loaded with a 0.5% (by weight) caffeine solution) over a 24-hour period in vitro under occlusive conditions.

FIG. 15E is a graphical representation of the caffeine flux that corresponds to the permeation profiles of FIG. 15A.

FIG. 15F is a graphical representation of the caffeine flux that corresponds to the permeation profiles of FIG. 15B.

FIG. 15G is a graphical representation of the caffeine flux that corresponds to the permeation profiles of FIG. 15C.

FIG. 15H is a graphical representation of the caffeine flux that corresponds to the permeation profiles of FIG. 15D.

FIG. 16A shows the cumulative amount of caffeine released from an embodiment of the invention (each including a hydrogel having been loaded with a 2% (by weight) caffeine solution) after a 0.5-hour application period as compared to a 1-hour application period in vitro under both non-occlusive and occlusive conditions. The notation “N.O.” refers to an application under non-occlusive conditions, whereas the notation “O.” refers to an application under occlusive conditions.

FIG. 16B shows the cumulative amount of caffeine released from an embodiment of the invention (each including a hydrogel having been loaded with a 2% (by weight) caffeine solution) after a 0.5-hour application period as compared to a 1-hour application period in vitro under both non-occlusive and occlusive conditions. The notation “N.O.” refers to an application under non-occlusive conditions, whereas the notation “O.” refers to an application under occlusive conditions.

FIG. 17A shows the permeation profiles of lidocaine as released from three different embodiments of the invention (each includes a hydrogel having been loaded with a 1%, 2%, and 5% (by weight) lidocaine solution, respectively) over a 24-hour period in vitro under occlusive conditions.

FIG. 17B shows the cumulative amount of lidocaine that was delivered to the epidermis and dermis, alone and combined, at the end of the 24-hour period described for FIG. 17A.

FIG. 18A shows the permeation profiles of lidocaine as released from five different embodiments of the invention (each includes a hydrogel having been loaded with either a 1% or 5% (by weight) lidocaine solution and having a pH of 3.0, 5.5, and 7.0, respectively) over a 24-hour period in vitro under occlusive conditions.

FIG. 18B shows the cumulative amount of lidocaine that was delivered to the epidermis and dermis, alone and combined, at the end of the 24-hour period described for FIG. 18A.

FIG. 19A shows the cumulative amount of lidocaine that was delivered by an embodiment of the invention (each includes a hydrogel having been loaded with a 2% (by weight) lidocaine solution and having a pH of 3.0) to the epidermis, dermis, and receptor medium in vitro under occlusive conditions after an application period of 15 minutes, 30 minutes, 1 hour, and 2 hours, respectively.

FIG. 19B shows the cumulative amount of lidocaine that was delivered by an embodiment of the invention (each includes a hydrogel having been loaded with a 2% (by weight) lidocaine solution and having a pH of 5.5) to the epidermis, dermis, and receptor medium in vitro under occlusive conditions after an application period of 15 minutes, 30 minutes, 1 hour, and 2 hours, respectively.

FIG. 19C shows the cumulative amount of lidocaine that was delivered by an embodiment of the invention (each includes a hydrogel having been loaded with a 2% (by weight) lidocaine solution and having a pH of 7.0) to the epidermis, dermis, and receptor medium in vitro under occlusive conditions after an application period of 15 minutes, 30 minutes, 1 hour, and 2 hours, respectively.

FIG. 19D shows the cumulative amount of lidocaine that was extracted from the hydrogel and the washings after the 5-minute, 30-minute, 1-hour, and 2-hour applications described for FIG. 19A, expressed as a percentage of the applied dose.

FIG. 19E shows the cumulative amount of lidocaine that was extracted from the hydrogel and the washings after the 5-minute, 30-minute, 1-hour, and 2-hour applications described for FIG. 19B, expressed as a percentage of the applied dose.

FIG. 19F shows the cumulative amount of lidocaine that was extracted from the hydrogel and the washings after the 5-minute, 30-minute, 1-hour, and 2-hour applications described for FIG. 19C, expressed as a percentage of the applied dose.

FIG. 20A shows the cumulative amount of lidocaine that was delivered by an embodiment of the invention (each includes a hydrogel having been loaded with a 1% (by weight) lidocaine solution and having a pH of 3.0) to the epidermis, dermis, and receptor medium in vitro under occlusive conditions after an application period of 15 minutes, 30 minutes, 1 hour, and 2 hours, respectively.

FIG. 20B shows the cumulative amount of lidocaine that was delivered by an embodiment of the invention (each includes a hydrogel having been loaded with a 1% (by weight) lidocaine solution and having a pH of 5.5) to the epidermis, dermis, and receptor medium in vitro under occlusive conditions after an application period of 15 minutes, 30 minutes, 1 hour, and 2 hours, respectively.

FIG. 20C shows the cumulative amount of lidocaine that was delivered by an embodiment of the invention (each includes a hydrogel having been loaded with a 1% (by weight) lidocaine solution and having a pH of 7.0) to the epidermis, dermis, and receptor medium in vitro under occlusive conditions after an application period of 15 minutes, 30 minutes, 1 hour, and 2 hours, respectively.

FIG. 20D shows the cumulative amount of lidocaine that was extracted from the hydrogel and the washings after the 5-minute, 30-minute, 1-hour, and 2-hour applications described for FIG. 20A, expressed as a percentage of the applied dose.

FIG. 20E shows the cumulative amount of lidocaine that was extracted from the hydrogel and the washings after the 5-minute, 30-minute, 1-hour, and 2-hour applications described for FIG. 20B, expressed as a percentage of the applied dose.

FIG. 20F shows the cumulative amount of lidocaine that was extracted from the hydrogel and the washings after the 5-minute, 30-minute, 1-hour, and 2-hour applications described for FIG. 20C, expressed as a percentage of the applied dose.

FIG. 21A is a photographic representation of the initial appearance of a full thickness wound on a rat covered with an embodiment of the invention on day 0 of treatment.

FIG. 21B is a photographic representation of the full thickness wound of FIG. 21A on day 2 of treatment with an embodiment of the invention.

FIG. 21C is a photographic representation of the full thickness wound of FIG. 21A on day 4 of treatment with an embodiment of the invention.

FIG. 21D is a photographic representation of the full thickness wound of FIG. 21A on day 6 of treatment with an embodiment of the invention.

FIG. 22A is a photographic representation of the initial appearance of a full thickness wound on a rat covered with a commercially available wound dressing on day 0 of treatment.

FIG. 22B is a photographic representation of the full thickness wound of FIG. 22A on day 2 of treatment with a commercially available wound dressing.

FIG. 22C is a photographic representation of the full thickness wound of FIG. 22A on day 4 of treatment with a commercially available wound dressing.

FIG. 22D is a photographic representation of the full thickness wound of FIG. 22A on day 6 of treatment with a commercially available wound dressing.

FIG. 23A is a photographic representation of the initial appearance of a full thickness wound on a rat covered with another commercially available wound dressing on day 0 of treatment.

FIG. 23B is a photographic representation of the full thickness wound of FIG. 23A on day 2 of treatment with the other commercially available wound dressing.

FIG. 23C is a photographic representation of the full thickness wound of FIG. 23A on day 4 of treatment with the other commercially available wound dressing.

FIG. 23D is a photographic representation of the full thickness wound of FIG. 23A on day 6 of treatment with the other commercially available wound dressing.

FIG. 24A is a photographic representation of a 2 cm×2 cm full thickness wound on a pig covered with an embodiment of the invention on day 0 of treatment.

FIG. 24B is a photographic representation of the 2 cm×2 cm full thickness wound of FIG. 24A on day 4 of treatment with an embodiment of the invention.

FIG. 24C is a photographic representation of the 2 cm×2 cm full thickness wound of FIG. 24A on day 7 of treatment with an embodiment of the invention.

FIG. 24D is a photographic representation of the 2 cm×2 cm full thickness wound of FIG. 24A on day 10 of treatment with an embodiment of the invention.

FIG. 24E is a photographic representation of the 2 cm×2 cm full thickness wound of FIG. 24A on day 21 of treatment with an embodiment of the invention.

FIG. 25A is a photographic representation of a 2 cm×2 cm full thickness wound on a pig covered with a commercially available wound dressing on day 0 of treatment.

FIG. 25B is a photographic representation of the 2 cm×2 cm full thickness wound of FIG. 25A on day 4 of treatment with a commercially available wound dressing.

FIG. 25C is a photographic representation of the 2 cm×2 cm full thickness wound of FIG. 25A on day 7 of treatment with a commercially available wound dressing.

FIG. 25D is a photographic representation of the 2 cm×2 cm full thickness wound of FIG. 25A on day 10 of treatment with a commercially available wound dressing.

FIG. 26A is a photographic representation of a 1 cm diameter full thickness wound on a pig covered with an embodiment of the invention on day 0 of treatment.

FIG. 26B is a photographic representation of the 1 cm diameter full thickness wound of FIG. 26A on day 4 of treatment with an embodiment of the invention.

FIG. 26C is a photographic representation of the 1 cm diameter full thickness wound of FIG. 26A on day 7 of treatment with an embodiment of the invention.

FIG. 26D is a photographic representation of the 1 cm diameter full thickness wound of FIG. 26A on day 10 of treatment with an embodiment of the invention.

FIG. 26E is a photographic representation of the 1 cm diameter full thickness wound of FIG. 26A on day 21 of treatment with an embodiment of the invention.

FIG. 27A is a photographic representation of a 1 cm diameter full thickness wound on a pig covered with a commercially available wound dressing on day 0 of treatment.

FIG. 27B is a photographic representation of the 1 cm diameter full thickness wound of FIG. 27A on day 4 of treatment with a commercially available wound dressing.

FIG. 27C is a photographic representation of the 1 cm diameter full thickness wound of FIG. 27A on day 7 of treatment with a commercially available wound dressing.

FIG. 27D is a photographic representation of the 1 cm diameter full thickness wound of FIG. 27A on day 10 of treatment with a commercially available wound dressing.

FIG. 28A is a photographic representation of a partial thickness wound on a pig covered with an embodiment of the invention on day 0 of treatment.

FIG. 28B is a photographic representation of the partial thickness wound of FIG. 28A on day 4 of treatment with an embodiment of the invention.

FIG. 28C is a photographic representation of the partial thickness wound of FIG. 28A on day 7 of treatment with an embodiment of the invention.

FIG. 28D is a photographic representation of the partial thickness wound of FIG. 28A on day 10 of treatment with an embodiment of the invention.

FIG. 29A is a photographic representation of a partial thickness wound on a pig covered with a commercially available wound dressing on day 0 of treatment.

FIG. 29B is a photographic representation of the partial thickness wound of FIG. 29A on day 4 of treatment with a commercially available wound dressing.

FIG. 29C is a photographic representation of the partial thickness wound of FIG. 29A on day 7 of treatment with a commercially available wound dressing.

FIG. 29D is a photographic representation of the partial thickness wound of FIG. 29A on day 10 of treatment with a commercially available wound dressing.

FIG. 30A is a photographic representation of the initial appearance of a 1 cm diameter chemical burn and a 1 cm diameter thermal burn before treatment.

FIG. 30B is a photographic representation of the 1 cm diameter chemical and thermal burns of FIG. 30A on day 4 of treatment with an embodiment of the invention.

FIG. 30C is a photographic representation of the 1 cm diameter chemical and thermal burns of FIG. 30A on day 10 of treatment with an embodiment of the invention.

FIG. 31A is a photographic representation of the initial appearance of a 1 cm diameter chemical burn and a 1 cm diameter thermal burn before treatment.

FIG. 31B is a photographic representation of the 1 cm diameter chemical and thermal burns of FIG. 31A on day 4 of treatment with a commercially available wound dressing.

FIG. 31C is a photographic representation of the 1 cm diameter chemical and thermal burns of FIG. 31A on day 10 of treatment with a commercially available wound dressing.

FIG. 32A is a photographic representation of the initial appearance of a surgical incision on a pig before treatment.

FIG. 32B is a photographic representation of the surgical incision of FIG. 32A on day 4 of treatment with an embodiment of the invention.

FIG. 32C is a photographic representation of the surgical incision of FIG. 32A on day 7 of treatment with an embodiment of the invention.

FIG. 32D is a photographic representation of the surgical incision of FIG. 32A on day 10 of treatment with an embodiment of the invention.

FIG. 33A is a photographic representation of the initial appearance of a surgical incision on a pig before treatment.

FIG. 33B is a photographic representation of the surgical incision of FIG. 33A on day 4 of treatment with a commercially available wound dressing.

FIG. 33C is a photographic representation of the surgical incision of FIG. 33A on day 7 of treatment with a commercially available wound dressing.

FIG. 33D is a photographic representation of the surgical incision of FIG. 33A on day 10 of treatment with a commercially available wound dressing.

FIG. 34A is a photographic representation of the initial appearance of certain lacerations on a human before treatment.

FIG. 34B is a photographic representation of the lacerations of FIG. 34A after 24 hours of treatment with an embodiment of the invention.

FIG. 34C is a photographic representation of the lacerations of FIG. 34A after 48 hours of treatment with an embodiment of the invention.

FIG. 35A is a photographic representation of the initial appearance of certain lacerations on a human before treatment.

FIG. 35B is a photographic representation of the lacerations of FIG. 35A after 72 hours of treatment with an embodiment of the invention.

FIG. 36A is a photographic representation of the initial appearance of a burn on a human before treatment.

FIG. 36B is a photographic representation of the burn of FIG. 36A after 48 hours of treatment with an embodiment of the invention.

FIG. 37A is a photographic representation of the initial appearance of an infected wound on a human before treatment.

FIG. 37B is a photographic representation of the infected wound of FIG. 37A after 48 hours of treatment with an embodiment of the invention as covered by an embodiment of the invention.

FIG. 37C is a photographic representation of the infected wound of FIG. 37A after 48 hours of treatment with an embodiment of the invention.

FIG. 37D is a photographic representation of the infected wound of FIG. 37A after 13 days of treatment with an embodiment of the invention.

FIG. 38A is a photographic representation of the initial appearance of certain wounds on a human with Ehlers-Danlos Syndrome before treatment.

FIG. 38B is a photographic representation of the wounds of FIG. 38A after 10 days of treatment with an embodiment of the invention.

FIG. 38C is a photographic representation of the wounds of FIG. 38A after 20 days of treatment with an embodiment of the invention.

FIG. 38D is a photographic representation of the wounds of FIG. 38A after 28 days of treatment with an embodiment of the invention.

FIG. 38E is a photographic representation of the wounds of FIG. 38A after 38 days of treatment with an embodiment of the invention.

FIG. 39A is a photographic representation of the initial appearance of a wound on the heel of a human with Ehlers-Danlos Syndrome before treatment.

FIG. 39B is a photographic representation of the wound of FIG. 39A after 10 days of treatment with an embodiment of the invention.

FIG. 39C is a photographic representation of the wound of FIG. 39A after 20 days of treatment with an embodiment of the invention.

FIG. 40A is a photographic representation of the initial appearance of a wound on the knee of a human with Ehlers-Danlos Syndrome before treatment.

FIG. 40B is a photographic representation of the wound of FIG. 40A after 10 days of treatment with an embodiment of the invention.

FIG. 40C is a photographic representation of the wound of FIG. 40A after 20 days of treatment with an embodiment of the invention.

### DETAILED DESCRIPTION OF THE INVENTION

The present invention provides a medical article that includes a hydrophilic water-swellable hydrogel having a crosslinked mixture of a biocompatible polymer and a protein. Hydrogels useful for this invention generally are prepared by crosslinking a protein with a bifunctionalized polymer to form a water-insoluble three-dimensional reticulated matrix, the integrity of which is reinforced by the physical interactions between the protein, the polymer, and if swollen, bound water molecules. As used herein, the singular forms “a,” “an,” and “the” include plural referents unless the context clearly dictates otherwise. Thus, for example, reference to “a protein” refers not only to a single protein but also to a mixture of two or more proteins, “a biocompatible polymer” refers not only to one type of biocompatible polymer but also to blends of biocompatible polymers and the like.

The hydrogels described herein may be produced from any hydrophilic polymers, including various homopolymers, copolymers, or blends of polymers that are biocompatible. As used herein, the term “biocompatible polymer” is understood to mean any polymer that does not appreciably alter or affect in any adverse way the biological system into which it is introduced. Illustrative of the biocompatible polymers that may be used are poly(alkylene oxide), poly(vinyl pyrrolidone), polyacrylamide, and poly(vinyl alcohol). Polyethylene oxide, such as polyethylene glycol (PEG), is particularly useful. Hydrophilic polymers useful in the applications of the invention include those incorporating and binding high concentrations of water while maintaining adequate surface tack (adhesiveness) and sufficient strength (cohesiveness). The starting polymer should have a molecular weight high enough, such that once reacted with the protein, it readily crosslinks and forms a viscous solution for processing. Generally, polymers with weight average molecular weights from about 0.05 to about 10×10⁴ Daltons, preferably about 0.2 to about 3.5×10⁴ Daltons, and most preferably, about 8,000 Daltons are employed.

Hydrogels included in the medical articles of the invention typically contain a significant amount of PEG crosslinked with a protein. The protein typically is an albumin. The protein may be obtained from a variety of sources including vegetal sources (e.g., soybean or wheat), animal sources (e.g., milk, egg, or bovine serum), and marine sources (e.g., fish protein or algae). An albumin from a vegetal source may be used (e.g., soybean), such that the hydrogel may be prepared at a minimal cost. Vegetal proteins are easily obtainable from different sources and therefore can be less expensive than animal-based proteins (e.g., bovine serum albumin) which have previously been used to make hydrogels. Additionally, proteins derived from vegetal sources are free of the prions and viruses that may be present in blood-derived proteins, such as BSA. These features make vegetal proteins desirable in the large-scale production of hydrogels suitable for use with the invention. The abundant charge groups on these proteins also provide additional water-retaining capacity in the hydrogel structure.

Typically, the water content of the hydrogels is greater than about 95% (w/w) based on the dry weight of the hydrogel as described in Example 11 below. The medical articles of the invention, therefore, are highly swellable. Additionally, it was observed that the hydrogels are capable of maintaining and inducing a moist environment, which is known to promote wound healing. As described in Example 14 below, the medical articles of the present invention may include a hydrating component composed of the hydrogels described herein.

To effect covalent attachment of PEG to a protein, the hydroxyl end-groups of the polymer are first converted into reactive functional groups. This process is frequently referred to as “activation” and the resulting bifunctionalized polyethylene oxide may be described by the general formula 1:
X—O—(CH₂CH₂O)ₙ—X (1)
where X can be any functional group able to react with the various chemical groups commonly found in proteins, including amino, thiol, hydroxyl, carboxyl, and carboxylic group, and n can vary from about 45 to about 800, which corresponds to commercial PEG of molecular weight ranging from about 2,000 to about 35,000 Daltons.

Several chemical procedures have been developed for the preparation of activated PEGs, which then can be used to react specifically with free amino groups of proteins. For example, PEGs have been successfully activated by reaction with 1,1-carbonyl-di-imidazole, cyanuric chloride, tresyl chloride, 2,4,5-trichlorophenyl chloroformate or p-nitrophenyl chloroformate, various N-hydroxy-succinimide derivatives, by the Moffatt-Swern reaction, as well as with various diisocyanate derivatives (Zalipsky S. (1995) BIOCONJUGATE CHEM. 6: 150-165 and references therein; Beauchamp et al. (1983) ANAL. BIOCHEM. 131: 25; Nashimura et al. (1983) LIFE SCI. 33: 1467; Delgado et al. (1990) APPL. BIOCHEM., 12: 119; Wirth et al. (1991) BIOORG. CHEM. 19: 133; Veronese et al. (1985) BIOCHEM. BIOTECHNOL. 11: 141; Sartore et al. (1991) BIOCHEM. BIOTECHNOL. 27: 45; Anderson et al. (1988) J. IMMUNOL. METHODS 109: 37; Zalipsky et al. (1990) J. BIOACT. COMPAT. POLYM. 5: 227; and U.S. Pat. No. 6,773,703).

The activation of PEGs with p-nitrophenyl chloroformate to generate PEG-dinitrophenyl carbonates has been described in U.S. Pat. No. 5,733,563 and by Fortier and Laliberte (Fortier et al. (1993) BIOTECH. APPL. BIOCHEM. 17: 115-130). This reaction is carried out in acetonitrile containing triethylamine (TEA) over a period of 5 hours at 60° C.

International Publication Number WO 03/018665 describes an alternative method for preparing activated PEGs with p-nitrophenyl chloroformate. The method involves a reaction carried out at room temperature using an aprotic solvent, such as methylene chloride (CH₂Cl₂), in the presence of a catalyst, such as dimethylaminopyridine (DMAP). Commercial PEG-dinitrophenyl carbonates suitable for preparing hydrogels included in the medical articles of the invention are available from Shearwater Corp. (Huntsville, Ala.).

In certain embodiments, the PEG forming the hydrogel is activated with p-nitrophenyl chloroformate and subsequently polymerized and crosslinked with a soy protein, e.g., soy albumin. The hydrogels so formed have useful physiological, mechanical, and optical properties—including a zero irritation index, a low sensitization potential, high water content, hydrophilicity, oxygen-permeability, viscoelasticity, moderate self-adhesiveness, translucidity, and controlled release of medications or drugs—that make them suitable for pharmaceutical, medical, and cosmeceutical applications. To achieve hydrogels having consistencies suitable for different applications, the plasticity and/or elasticity of the hydrogels may be modified by varying the amounts of PEG and protein used to synthesize the hydrogels, the molecular weight of the PEG used, or the nature of the protein used.

The hydrogels may include a buffer system to help control the pH, to prevent discoloration and/or breakdown due to hydrolysis. Suitable buffers include, but are not limited to, sodium potassium tartarate and/or sodium phosphate monobasic, both of which are commercially readily available from, for example, Sigma-Aldrich Chemical Co. (Milwaukee, Wis.). In certain embodiments, the hydrogel may be loaded with a buffer solution to adjust the pH of the hydrogel within the range of 3.0-9.0. In some embodiments, an acid or a base may be used instead of the buffer solution for the same purpose. The use of a buffer system provides the hydrogels with a commercially suitable shelf-life, allowing some hydrogels described herein to be stored for at least six months (e.g., in a 10 mM phosphate-EDTA buffer at 4° C. without any changes to their properties).

To ensure that the hydrogels are sterile, the hydrogels may be prepared in a clean room and/or suitable preservatives and/or antimicrobial agents may be incorporated into the hydrogels. A preservative having antimicrobial properties sold under the name of LIQUID GERMALL® PLUS (International Specialty Products, Wayne, N.J.) is particularly useful. The LIQUID GERMALL® PLUS preservative has been incorporated into cosmetic products and contains propylene glycol (60 wt. %), diazolidinyl urea (39.6 wt. %), and iodopropynyl butylcarbamate (0.4 wt. %). Throughout the remainder of the text, reference to LIQUID GERMALL® PLUS refers to this described composition.

Other additives, including colorants, fragrance, binders, plasticizers, stabilizers, fire retardants, cosmetics, and moisturizers, may also be optionally present. These ingredients may be added into either one of the protein or PEG solutions before polymerization. Alternatively, additives may be loaded into the hydrogel after it has been formed and optionally dried. In either case, the additives typically are uniformly dispersed within the hydrogel. These additives may be present in individual or total amounts of about 0.001 to about 6 weight percent of the total mixture, preferably not exceeding about 3 weight percent in the final hydrogel.

Further, the physical appearance of hydrogels may be modified depending on the application. For example, hydrogels may be prepared in different forms (such as films, discs, block, etc.) by pouring the hydrogel solution between glass plates or in a plastic mold. Once set, the hydrogel may be cut into pellets or pastilles, shredded into fibers, or broken up to form particles of difference sizes. Particles also could be made by suspension or emulsion polymerization.

Hydrogel-containing medical articles of the invention typically do not represent a limiting factor for short-term drug-delivery. The medical articles described herein also do not represent a limiting factor for long-term drug-delivery if applied under occlusive conditions (as described in Example 17 below). Therefore, the incorporation of pharmaceutically active agents into the hydrogels described above may impart desirable pharmaceutical activities. As in the case with additives, the pharmaceutically active agents may be incorporated before or after polymerization with protein. For simplicity of production and economy of scale, however, typically, the pharmaceutically active agents are prepared as a loading solution and loaded into preformed hydrogel blanks. Loading solutions may be buffered as described above to maintain the hydrogel and/or may contain stabilizing agents to maintain the active agent in an active and/or stable form.

As used herein, the term “pharmaceutically active agent” is used interchangeably with the terms “drug,” “active agent,” “active ingredient,” “active,” and “agent” and is intended to have the broadest interpretation as to any element or compound which has an effect on the biochemistry or physiology of a mammal or other organism (e.g., a microbe). The pharmaceutically active agent may, for example, have a therapeutic or diagnostic effect. Typical pharmaceutically active agents include, for example, antimicrobial agents (e.g., LIQUID GERMALL® PLUS), analgesic agents (e.g., aspirin), anti-inflammatory agents (e.g., naproxen), anti-itch agents (e.g., hydrocortisone), antibiotics (e.g., macrolides), healing agents (e.g., allantoin), anesthetics (e.g., benzocaine), and the like.

It is to be understood that any therapeutically-effective amount of active ingredient that may be loaded into the hydrogels of the medical articles of the invention may be employed, with the proviso that the active ingredient does not substantially alter the crosslinking structure of the hydrogel. Typically, the drugs are water-soluble. As used herein, the term “therapeutically-effective amount” refers to the amount of an active agent sufficient to induce a desired biological result. That result may be alleviation of the signs, symptoms, or causes of a disease, or any other desired alteration of a biological system. Such pharmaceutically active agents are typically present in an amount of from about 0.01 to about 50 weight percent, although higher and lower concentrations are within the scope of the present invention.

Table 1 provides non-limiting examples of active ingredients that may be incorporated into the hydrogel of the present invention. Table 2 provides exemplary dosages of certain drugs.

TABLE 1
Exemplary list of drugs for inclusion in a medical article.

DRUG
Acetazolamide, Sodium
Alphaprodine HCl
Amicocaproic Acid
Aminosuppurate Sodium
Aminophylline
Aminotryptyline HCl
Amobarbitol Sodium
Anileridine
Amphotericin B
Ampicillin
Anti coagulant Heparin Solution
Arginine HCl
Atropine Sulfate
Atrial Peptides
Azathioprine Sodium
Benztropine Mesylate
Betaine HCl
Betamathazone Sodium
Bethanecol Chloride
Biperiden Lactate
Bleomycin Sulfate
Brompheniramine Maleate
Bupivacaine-Epinephrine Injection
Bupivacaine HCl
Butabartitol Sodium
Butorphanol Tartrate
Caffeine-Sodium Benzoate Injection
Calcium Glueptate Injection
Calcium Levulinate
Carboprost Tromethiamine Injection
Cefamandole Sodium
Cefamandole Nafate
Caphazolin Sodium
Cafataxime Sodium
Ceftizoxime Sodium
Cephalothin Sodium
Caphaprin Sodium
Caphradine
Cafonocid Sodium
Chloramphenicol
Chlordiazepoxide HCl
Chloroprocaine HCl
Chlorothiazide Sodium
Chlorpromazine HCl
Cefoperazone Sodium
Chlorphenramine Maleate
Chloroquine HCl
Chlortetracycline NCl Clorprothixene
Colohicine Desmopressin
Clindamycin Phosphate
Cimetadine Hydrochloride
Codeine Phosphate
Corticotropin
Cyanocobalamin
Cyclizine Lactate
Cyclophosphamide
Cyclosporine
Cysteine HCl
Chlorprothixene HCl
Dantrolene Sodium
Dacarbazine
Cactinomycin
Daumorubicin HCl
Deslanoside
Desmopressin Acetate
Dexamethasone Sodium Phosphate
Diatrizoate Meglumine
Diatrizoate Sodium
Diazepam
Diazolidinyl Urea
Diazoxide
Dibucaine HCl
Dicyclomine HCl
Diethylstilbesterol Diphosphate
Digoxin
Dihydroergotamine Mesylate
Diphenhydramine HCl
Dimenhydrinate
Dobutamine HCl
Dopamine HCl
Dopamine HCl-Dextrose
Doxapram HCl
Doxorubicin HCl
Droperidol
Dhphylline
Edetate Disodium
Emetine HCl
Ephedrine Sulfate
Epinephrine
Ergonovine Maleate
Ergotamine Tartrate
Erythromycin
Erythromycin Ethylsuccinate
Erythromycin Gluceptate Erythromycin
Lactibionate
Estradiol Valerate
Ethacrynate Sodium thylnorepinephrine
HCl
Etidocaine HCl
Fentanyl Citrate
Floxuridine
Fluorescein Sodium
Fluoracil
Fluphenazine Enanthate Fluphenazine
HCl
Folic Acid
Furosemide
Fallamine Triethiodide
Gentamycin Sulfate
Glucagon
Glycopyrrolate
Haloperidol
Heparin-Calcium
Heparin-Sodium
Hetacillin-Potassium Hexafluorenium
Bromide
Histamine Phosphate
Hyaluranidase
Digitoxin
Fructose
Hydralazine HCl
Hydrocortisone Sodium Phosphate
Hydrocortisone Sodium Succinate
Hydromorphone HCl
Hydoxocobalamin
Hydroxyzine HCl
Hyoscyamine Sulfate
Imipramine HCl
Iodopropynyl Butylcarbamate
Iophendylate
Iothalamate Sodium
Iron Dextran
Isobucaine HCl-Epinephrine
Isoniazid
Isoproterenol HCl
Isoxsuprine HCl
Kanamycin Sulfate
Ketamine HCl
Leucovorin Calcium
Levallorphan Tartrate
Lidocaine HCl
Lidocaine HCl Dextrose
Lidocaine HCl-Epinephrine
Lidocaine HCl-Epinephrine Bitartrate
Lincomycin HCl
Magnesium Sulfate
Magnesium Chloride
Methlorethamine HCl
Menotropins
Meperidine HCl
Mephentermine Sulfate
Mepivacaine HCl
Mepivacaine HCl-Levonordefrin
Meprylcaine HCl-Epinephrine
Mesoridazine Besylate
Metaraminol Bitartrate
Methadone HCl
Methicillin Sodium
Methiodal Sodium
Methocarbamol
Methohexital Sodium
Methotrexate Sodium
Methotrimeprazine
Methoxamine HCl
Methscopolamine Bromide
Methyldopate HCl
Methylergonovine Maleate
Methylpredisolone Sodium Succinate
Metronidazone
Miconazole
Minocycline HCl
Mitomycin
Morphine Sulfate
Moxalactam Disodium
Nafcillin Sodium
Naloxone HCl
Neostigmine Methylsulfate
Netilmicin Sulfate
Niacin
Niacinamide
Norepinephrine Bitartrate
Nylidrin HCl
Orphenadrine Citrate
Oxacillin Sodium
Oxymorphone HCl
Oxytetracycline
Oxytetracycline HCl
Oxytocin
Papaverine HCl
Parathyroid
Penicillin G Potassium
Penicillin G Procaine
Penicillin G Sodium
Pentazocine Lactate
Phenobarbital Sodium
Perphenazine
Phenobarbitol Sodium
Phentolamine Mesylate Phenylephrine
HCl
Phenytoin Soidum
Physopstigmine Salicylate
Phytonadione
Plicamycin
Posterior Pituitary
Potassium Acetate Potassium Chloride
Prednisolone Sodium Phosphate
Prednisolone Sodium Succinate
Prilocaine HCl
Procainamide HCl
Procaine HCl
Procaine HCl-Epinephrine
Procaine-Phsnylephrine
Hydrochlorides
Procaine and Tetracaine HCl
and Levonodefrin
Prochlorperazine Edisylate
Promazine HCl
Promethazine HCl Propiomazine HCl
Propoxycaine-Procaine HCl
Norepinephrine Bitartrate
Propanolol HCl
Protein Hydrolysate
Pyridostigmine Bromide
Pyridoxine HCl
Quinidine Gluconate
Reserpine
Riboflavin
Ritodrine HCl
Rolitetracycline
Scopolamine HCl
Secobarbital Sodium
Sisomycin Sulfate
Spectinomycin HCl
Streptomycin Sulfate
Succinylcholine Chloride
Sulfadixazine Sodium
Sulfixoxazole Diolamine
Superoxide Dismutase
Terbutaline Sulfate
Testosterone Cypionate
Testosterone Enanthate
Tetracaine HCl
Tetracycline HCl
Tetracycline Phosphate Complex
Thiamine HCl
Thimylal Sodium
Thiethylperazine Maleate
Thiopental Sodium
Thiothixene HCl
Tobramycin Sulfate
Tolazoline HCl
Tolbutaminde Sodium
Triamcinolane Diacetate
Tridihexethyl Chloride
Trifluoperazine HCl
Triflupromzine HCl
Trimethaphan Camsylate
Trimethobenzamide HCl
Trimethoprimsulfamethoxazole
Tromethamine
Tubocurarine Chloride
Vasopressin
Vincristine Sulfate
Vidarabine Concentrate
Vinclastine Sulfate
Warfarin Sodium
Verapamil

TABLE 2
Examples of drug in standard dosage forms.

| DRUG | DOSAGE | |
|---|---|---|
| Cimetidine HCl | 150 | mg/ml |
| Diazepam | 5 | mg/ml |
| 5-Fluorouracil | 500 | mg/ |
| | 10 | ml |
| Erythromycin Lactobionate | 1 | mg/ml |
| Flosuridine | 500 | mg/ |
| | 5 | ml |
| Amthoteracin D | 0.1 | mg/ml |
| Fluphenazine HCl | 2.5 | mg/ml |
| Heparin Sodium | 1,00-20,000 | units/ml |
| Haloperidol lactate | 5 | mg/ml |
| Insulin | 40 | units |
| Ketamine HCl | 10 | mg/ml |
| Labeltol HCl | 5 | mg/ml |
| Lipocaine HCl | 10 | mg/ml |
| Miconazole | 10 | mg/ml |
| Morphine Sulfate | 0.5-1.0 | mg/ml |
| Dropendal | 2.5 | mg/ml |
| Imipramine HCl | 25 | mg/ |
| | 2 | ml |
| Phenytoin | 100 | mg/ml |
| Pentobartital Sodium | 50 | mg/ml |
| Tetracycline HCl | 250 | mg/ |
| | 100 | ml |
| Thiopental Sodium | 0.2 | mg/ |
| | 2 | ml |
| Verapamil HCl | 2.5 | mg/ml |
| Vincristine Sulfate | 1.0 | mg/ml |
| Fentanyl citrate | 0.05 | mg/ml |
| Succinate | 40 | mg/ml |

As described above, antimicrobial agents may be incorporated into the hydrogel to keep it sterile. Depending on the concentration of the antimicrobial agents, the hydrogel may further be imparted antimicrobial properties, in addition to maintaining sterility as described above. As used herein, the term “antimicrobial properties” refers to a hydrogel that exhibits one or more of the following properties—the inhibition of the adhesion of bacteria and/or other microbes to the hydrogel, the inhibition of the growth of bacteria and/or other microbes on the surface of the hydrogel and/or within the hydrogel matrix, and the killing of bacteria and/or other microbes on the surface of the hydrogel, within the hydrogel matrix and/or in an area extending from the hydrogel. Medical articles containing hydrogels as described herein can provide at least a 1-log reduction (greater than 90% inhibition) of viable bacteria or other microbes, and more preferably, about a 2-log reduction (greater than 99% inhibition) of viable bacteria or other microbes in in vitro tests. Such bacteria or other microbes include, but are not limited to, those organisms found on the skin, particularly Candida albicans, Aspergillus niger, Staphylococcus aureus, Bacillus cereus, Escherichia coli, and Pseudomonas aeruginosa.

Specific examples of antimicrobial agents used in the present invention include various bactericides, fungicides, and antibiotics that are effective against a broad spectrum of microbes without causing skin irritation. In certain embodiments, non-antibiotic antimicrobial agents are employed, to avoid developing antibiotic-resistant microbes. Suitable non-antibiotic antimicrobial agents include, but are not limited to, diazolidinyl urea, quaternary ammonium compounds (e.g., benzalkonium chloride), and various oxidizing agents including, but not limited to, biguanides (e.g., chlorhexidine digluconate), silver compounds (e.g., silver sulphadiazine), and iodine-containing compounds (e.g., iodopropynyl butylcarbamate). In certain embodiments, the hydrogels are imparted antimicrobial properties by loading with LIQUID GERMALL® PLUS, a combination of diazolidinyl urea and iodopropynyl butylcarbamate, diazolidinyl urea alone or in combination with other actives, and/or iodopropynyl butylcarbamate alone or in combination with other actives.

In some embodiments, the medical article may further include a support or a backing which may or may not be adhesive to an application site or have an adhesive applied thereto. The support or backing may include a polymeric surface to which the hydrogel is attached. The backing may be made adhesive to the hydrogel by exposing the surface of the polymeric backing to an activated gas as described in International Application Publication No. WO02/070590. Specifically, a polymeric backing, such as polyethylene terephthalate, can be exposed to plasma of various gases or mixture of gases, including, but not limited to, nitrogen, ammonia, oxygen, and various noble gases, produced by an excitation source such as microwave and radiofrequency. A polymeric backing so treated typically adheres to the hydrogels used with the medical articles according to the invention.

In some embodiments, the medical article may include multiple supports. For example, the hydrogel may be present in a first layer and the support may be present in a second layer, and the medical article may include a plurality of alternating first and second layers.

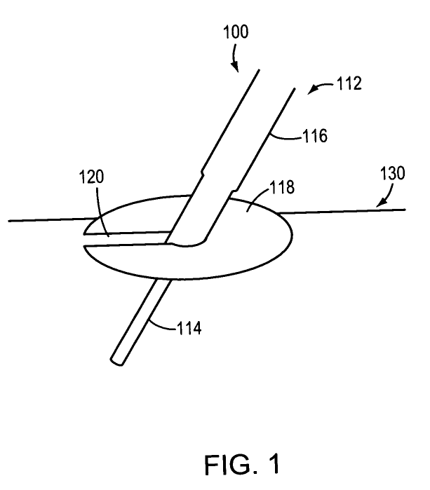

In other embodiments, and with reference to FIG. 1, the medical article 100 may include an in-dwelling member 112, such as a catheter. The in-dwelling member may include a first portion 114 which is adapted to be inserted into the body of a patient and a second portion 116 which is adapted to be exposed outside the body of a patient. The hydrogel 118 may include a longitudinal slot 120 or an opening of any shape. The shape of the opening is not critical, as long as it is dimensioned and sized to be compatible with the in-dwelling member such that at least the second portion of the in-dwelling member may lie within or pass through the opening in the hydrogel. The hydrogel may be provided together with the in-dwelling member or separately therefrom. In some embodiments, the hydrogel may be disposed at or around a topical site 130 of the patient, the topical site being the entry site of the in-dwelling member. Furthermore, medical articles including the hydrogels described above may be used at any anatomical site where a medical instrument enters the body (e.g., punctures a barrier or enters a cavity). For example, the medical articles may be used as an antimicrobial barrier on a skin insertion site where the skin is punctured or where a medical article is inserted into a patient's urethra at the interface between the environment and the patient's inner body.

Administration of the medical articles of the present invention to a wound or puncture site can result in accelerated wound repair with reduced or no sepsis, as described in Example 18 below. Even with wounds that penetrate the dermal layer, there can be reduced pain sensation, more extensive and quicker tissue growth, and less overall discomfort to the patient. An additional benefit is that the tissue repair induced by the hydrogels restricts opportunistic infections that would otherwise prolong the period of wound healing, increase the extent of the wound, or even develop to threaten the life of the infected patient. Furthermore, the hydrogels may be loaded with active agents to prevent and/or treat any infected wounds.

When using any of the medical articles of the invention, the medical articles can be applied to an anatomical site. This site can be an open wound or an intact anatomical site (e.g., the skin). The medical article then resides on the surface to which it is applied. The medical article may remain in place on the surface because of its inherent properties (e.g., tackiness) or, alternatively, may have an adhesive applied to it. Suitable adhesives include any medically accepted, skin friendly adhesive, including acrylic, hydrocolloid, polyurethane and silicone-based adhesives. To the extent the medical article is used to treat a wound, it is placed over all or a portion of the wound. Actives may be incorporated into the hydrogel of the medical article to assist in healing the wound, prevent and/or inhibit infection, and/or diminish the pain associated with the wound. Alternatively, any of the medical articles of the invention can be used as a drug delivery “patch.” Actives resident within the hydrogel may be delivered topically or systematically, for example to or through the skin. Skin permeation enhancers may be added to the medical article, if desired, to enhance the delivery of an active.

Medical articles of the invention are suitable for a wide range of applications. Exemplary uses include wound dressings or artificial skins, solid humidified reaction mediums for diagnostic kits (for use in fundamental research such as PCR, RT-PCR, in situ hybridization, in situ labeling with antibodies or other markers such as peptides, DNA or RNA probes, medicaments or hormones), transport mediums (for cells, tissues, organs, eggs, or organisms), tissue culture mediums (with or without active agents), electrode materials (with or without enzymes), iontophoretic membranes, protective humidified mediums for tissue sections (such as replacement cover glasses for microscope slides), matrices for the immobilization of enzymes or proteins (for in vivo, in vitro, or ex vivo use as therapeutic agents, bioreactors or biosensors), cosmeceutical applications (such as skin hydrators or moisturizers), decontamination and/or sterilization means, and drug-release devices that could be used in systemic, intratumoral, subcutaneous, topical, transdermic and rectal applications.

For in vivo applications, the medical articles of the invention can be administered in a pharmaceutically acceptable form to any anatomical site of a vertebrate, including humans and animals. Illustrative anatomical sites include, but are not limited to, oral, nasal, buccal, rectal, vaginal, topical sites (e.g., skin, dermis, and epidermis), and any other anatomical sites where the application of the medical articles of the invention will bring forth a beneficial effect. In some embodiments, the medical articles are applied to an anatomical site that has been infected by microorganisms.

In other embodiments, the medical articles of the invention may be specifically designed for in vitro applications, such as disinfecting or sterilizing medical instruments and devices, contact lenses and the like, particularly when the devices or lenses are intended to be used in contact with a patient or wearer. For example, the medical articles may be used to decontaminate medical and surgical instruments and supplies prior to contacting a subject. Additionally, the medical articles may be used, post-operatively or after any invasive procedure, to help minimize the occurrence of post-operative infections. Also, the medical articles may be administered to subjects with compromised or ineffective immunological defenses (e.g., the elderly and the very young, burn and trauma victims, and those infected with HIV and the like).

In another aspect, the present invention provides methods for treating a wound. The methods include administering a first medical article to a wound, the first medical article being one of the medical articles described above, such that wound healing occurs faster as compared to a wound that is treated in an identical manner by a second medical article having a composition different from that of the first article. In some embodiments, the second medical article may be a wound dressing which includes a polyurethane membrane coated with a layer of an acrylic adhesive (e.g., a TEGADERM™ wound dressing, marketed by 3M). The rate of wound healing may be determined by measuring one or more criteria including reduction of wound size, amount of time to achieve wound closure, contrast between wound color and normal tissue color, signs of infection, and duration of the inflammatory phase.

As used herein, “healthy skin,” “normal tissue” or “normal skin” refers to non-lesional skin (i.e., with no visually obvious erythema, edema, hyper-, hypo-, or uneven pigmentations, scale formation, xerosis, or blister formation). Histologically, healthy or normal skin refers to skin tissue with a morphological appearance comprising well-organized basal, spinous, and granular layers, and a coherent multi-layered stratum corneum. In addition, the normal or healthy epidermis comprises a terminally differentiated, stratified squamous epithelium with an undulating junction with the underlying dermal tissue. Normal or healthy skin further contains no signs of fluid retention, cellular infiltration, hyper- or hypoproliferation of any cell types, mast cell degranulation, and parakeratoses and implies normal dendritic processes for Langerhans cells and dermal dendrocytes. This appearance is documented in dermatological textbooks, for example, Lever et al. eds. (1991) “Histopathology of the Skin,” J.B. Lippincott Company, PA; Champion et al. eds. (1992) “Textbook of Dermatology,” 5th Ed. Blackwell Scientific Publications, especially Chapter 3 “Anatomy and Organization of Human Skin;” and Goldsmith ed. (1991) “Physiology, Biochemistry, and Molecular Biology of the Skin,” Vols. I and II, Oxford Press.

The present invention further provides methods for treating both infected and non-infected wounds and treating and/or preventing an infection. The methods include applying to an anatomical site of a patient one of the medical articles described above. The medical article may include a hydrating component, such as a hydrophilic water-swellable hydro gel which includes a crosslinked mixture of a biocompatible polymer and a protein. The medical article may further include at least one of diazolidinyl urea and iodopropynyl butylcarbamate, or alternatively or in addition, another oxidizing agent, dispersed within the hydrogel, in a therapeutically effective amount to generate an antimicrobial effect. The medical article may be applied to a topical site which may include an open wound or which may be physically intact.

The present invention also provides methods for drug delivery. A medical article is loaded with an active and applied to an anatomical site of a patient. In certain embodiments, a region of epidermis of a patient can be hydrated (e.g., hyper-hydrated) and an active agent is provided to the hydrated region, thereby to deliver the agent cutaneously and/or percutaneously to the patient. For example, the region of epidermis is hydrated by applying one of the medical articles described above to that region and the active agent is delivered from within the hydrogel of the medical article. In some embodiments, a dry form of the hydrogel (obtained after dehydration under vacuum or in acetone) may be used. For example, the hydrogel firstly may be employed as a water or exudate absorbent in wound dressing, and secondly, as a slow or controlled drug release device.

Practice of the invention will be still more fully understood from the following example, which is presented herein for illustration only and should not be construed as limiting the invention in any way.

### EXAMPLE 1

### Activation of PEG Using P-Nitrophenyl Chloroformate Catalyzed by Triethylamine (TEA)

PEG of various molecular masses (n varying from 45 to 800) were activated using p-nitrophenyl chloroformate to obtain PEG dinitrophenyl carbonates (Fortier et al. (1993) BIOTECH. APPL. BIOCHEM. 17: 115-130). Before use, all PEGs had been dehydrated by dissolving 1.0 mmole of PEG in acetonitrile and refluxing at 80° C. for 4 hours in a Soxhlet™ extractor containing 2.0 g of anhydrous sodium sulfate. The dehydrated solution containing 1.0 mmole of PEG was activated in the presence of at least 3.0 mmoles of p-nitrophenyl chloroformate in acetonitrile containing up to 5 mmoles of TEA. The reaction mixture was heated at 60° C. for 5 hours. The reaction mixture was cooled and filtered and the synthesized PEG-dinitrophenyl carbonate (PEG-NPC₂) was precipitated by the addition of ethyl ether at 4° C. The percentage of activation was evaluated by following the release of p-nitrophenol (pNP) from the PEG-NPC₂ in 0.1M borate buffer solution, pH 8.5, at 25° C. The hydrolysis reaction was monitored at 400 nm until a constant absorbance was obtained. The purity was calculated based on the ratio of the amount of pNP released and detected spectrophotometrically versus the amount of pNP expected to be released per weight of PEG-NPC₂ used for the experiment. The purity of the final products was found to be around 90%.

### EXAMPLE 2

### Activation of PEG Using P-Nitrophenyl Chloroformate Catalyzed by Dimethylaminopyridine (DMAP)

PEG 8 kDa (363.36 g; 45 mmoles) was dissolved in anhydrous methylene chloride (CH₂Cl₂) (500 mL), and p-nitrophenyl chloroformate (19.63 g) was dissolved in anhydrous CH₂Cl₂ (50 mL). Both solutions were then added to a reaction vessel and stirred vigorously for about one minute. To this solution was then added a previously prepared DMAP solution (12.22 g of DMAP was dissolved in 50 mL of anhydrous CH₂Cl₂) while stirring was continued. The reaction mixture was then stirred for an additional 2 hours at room temperature.

The reaction mixture was concentrated and precipitated using diethyl ether (2.0 L) cooled to 4° C. The resulting suspension was then placed in a refrigerator (−20° C.) for a period of 30 minutes. The suspension was vacuum filtered and the precipitate washed several times with additional cold diethyl ether. The washed precipitate was then suspended in water, stirred vigorously for about 30 minutes, and vacuum filtered. The so-obtained yellow-like filtrate was then extracted three times with CH₂Cl₂ and the combined solvent fractions filtered over Na₂SO₄. The filtrate was concentrated and the resulting product was precipitated under vigorous stirring using cold diethyl ether. The PEG-NPC₂ so-obtained was then filtered, washed with diethyl ether, and dried under vacuum. The percentage of activation was evaluated by following the release of pNP from the PEG-NPC₂ in 0.1M borate buffer solution, pH 8.5, at 25° C. The hydrolysis reaction was monitored at 400 nm until a constant absorbance was obtained. The purity was calculated based on the ratio of the amount of pNP released and detected spectrophotometrically versus the amount of pNP expected to be released per weight of PEG-NPC₂ used for the experiment. The purity of the final products was found to be around 97%.

### EXAMPLE 3

### Solvent-Free Activation of PEG Using P-Nitrophenyl Chloroformate

PEG 8 kDa (Fischer Scientific, 300.0 g, 37.5 mmol) was placed in a vacuum flask equipped with a thermometer and a stirrer. Upon heating to 65-70° C., the PEG powder began to melt. Once the PEG powder was completely melted, portions of p-nitrophenyl chloroformate (ABCR GmbH & Co. KG, Karlsruhe, Germany) comprising 33% of the equimolar amount of the terminal OH groups of PEG were added to the molten PEG at 15-minute intervals until a 200% excess of p-nitrophenyl chloroformate was added in total. The reaction mixture was stirred at 70-75° C. for two hours, then kept under vacuum overnight to remove residual HCl vapors. The crystallized PEG-NPC₂ product was then ground into a powder and dissolved in water to prepare a crude PEG-NPC₂ solution. To remove free pNP, weighted amounts of activated carbon (about 5 to 15 wt. % of activated PEG) was added to the PEG-NPC₂ solution, followed by filtration. The filtered PEG-NPC₂ solution was subsequently subjected to lyophilization. NMR studies indicated that PEG-NPC₂ prepared by this method could achieve complete activation (i.e., 100% degree of activation) by using 67 mol % or more excess of the activator (i.e., p-nitrophenyl chloroformate).

### EXAMPLE 4

### Preparation of Hydrogels Using PEG-NPC₂ and Animal-Based Albumins

Covalent crosslinking of the PEG-NPC₂ to albumin of various sources, for example, from serum (e.g., bovine serum albumin), milk (lactalbumin) or egg (ovalbumin), was obtained by adding to one ml of 5% (w/v) protein solution (in either phosphate or borate buffer adjusted to pH 10.3) different amounts of PEG-NPC₂ (from 7 to 13% w/v) as prepared by any of the methods described in Examples 1 to 3, followed by vigorous mixing until all the PEG-NPC₂ powder was dissolved. The ratio of reagents (PEG/NH₂, the molar ratio of PEG activated groups versus albumin accessible NH₂ group) was determined taking into account that bovine serum albumin (BSA) has 27 accessible free NH₂ groups. The hydrogels obtained were incubated in 50 mM borate buffer, pH 9.8, in order to hydrolyze the unreacted PEG-NPC₂. The released pNP, the unreacted PEG-NPC₂, and the free proteins were eliminated from the gel matrix by washing the hydrogels in distilled water containing 0.02% NaN₃.

### EXAMPLE 5

### Preparation of Hydrogels Using PEG-NPC₂ and Casein

Casein (purchased from American Casein Company, Burlington, N.J.) was dissolved to a concentration of about 3% to about 9% (w/v) in an aqueous solution containing a strong inorganic base (such as NaOH, KOH, LiOH, RbOH and CsOH) or an organic base (such as triethylamine). This solution was combined with an aqueous solution of PEG-NPC₂ having a concentration ranging from about 3% to about 30% (w/v), which could be prepared by any of the methods described in Examples 1 to 3. The resulting solution was vigorously mixed until homogenization occurred.

Diluting the protein solution with a NaOH solution having an ionic strength that increased from about 0.12 N to about 0.20 N was found to decrease the gellification time from about 58 seconds to about 10 seconds.

The mixture was placed between two pieces of glass to form gel samples with a thickness of 1.8 mm. The resulting hydrogels were washed in EDTA/NaCl buffer to remove residual pNP and unreacted PEG and casein.

It was observed that the hydrogels prepared by this method were mechanically strong and showed good elasticity.

### EXAMPLE 6

### Preparation of Hydrogels Using PEG-NPC₂ and Soy Albumin

A weighted amount of PEG-NPC₂ (5.5 g) prepared by any of the methods described in Examples 1 to 3 was added to 25 mL of deionized water. Soy albumin was dissolved in 0.14N NaOH to give a 12% (w/v) (120 mg/mL) soy albumin solution, and the pH of the solution was adjusted to 11.80. The PEG-NPC₂ solution was mixed with the soy albumin solution using a SIM device. The mixture was placed between two pieces of glass to form gel samples with a thickness of 1.8 mm. The resulting hydrogels were washed in EDTA/NaCl buffer to remove residual pNP and unreacted PEG and soy albumin.

### EXAMPLE 7

### Preparation of Hydrogels Using PEG-NPC₂ and Hydrolyzed Soy Protein

A 10% (w/v) hydrolyzed soy protein solution was prepared by combining dry soy protein (purchased from ADM Protein Specialties, Decatur, Ill.) with distilled water followed by homogenizing in a blender. The temperature of the solution obtained was raised to 80° C. and 2.15 moles of HCl were added per kilogram of soy protein. The resulting solution was vigorously agitated for 4 hours at 80° C. and allowed to cool to room temperature. The pH of the solution was then increased to between 9 and 10 by adding NaOH while vigorous mixing was continued. The pH of the solution was subsequently lowered to about 4, and the precipitate obtained as a result of the lowering of the pH was collected by centrifugation at 2000 G for 10 minutes. The precipitate containing hydrolyzed soy protein was washed twice by removing the supernatant, mixing with an equivalent volume of distilled water, and centrifuging the solution obtained at 2000 G for 10 minutes. The final precipitate of hydrolyzed soy protein was dissolved in a volume of 1 to 5 mls distilled water per gram of soy protein and the solution was equilibrated to pH 7. The neutral solution was lyophilized to obtain a dry powder.

To covalently crosslink PEG-NPC₂ with the hydrolyzed soy protein, the hydrolyzed soy protein was dissolved to a concentration of about 8.0% to about 15.0% (w/v) in an aqueous solution containing a strong inorganic base (e.g., NaOH, KOH, LiOH, RbOH and CsOH) or an organic base (e.g., triethylamine). This solution was combined with an aqueous solution of PEG-NPC₂ having a concentration ranging from about 2% to about 30% (w/v), which could be prepared by any of the methods described in Examples 1 to 3. The resulting solution was vigorously mixed until homogenization occurred.

Diluting the protein solution with a NaOH solution having an ionic strength that increased from about 0.09 N to about 0.17 N was found to decrease the gellification time from about 60 seconds to about 20 seconds. Complete polymerization also took place faster.

The mixture was placed between two pieces of glass to form gel samples with a thickness of 1.8 mm. The resulting hydrogels were washed in EDTA/NaCl buffer to remove residual pNP and unreacted PEG and soy protein.

It was observed that the hydrogels prepared by this method were mechanically strong and showed good elasticity.

### EXAMPLE 8

### Preparation of Hydrogels Using PEG-NPC₂ and Hydrolyzed Wheat Protein

A 10% (w/v) hydrolyzed wheat protein solution was prepared by combining wheat protein (purchased from ADM Protein Specialties, Decatur, Ill.) with distilled water followed by homogenizing in a blender. The temperature of the solution obtained was raised to 80° C. and 2.15 moles of HCl were added per kilogram of wheat protein. The resulting solution was vigorously agitated for 4 hours at 80° C. and allowed to cool to room temperature. The pH of the solution was then increased to between 9 and 10 by adding NaOH while vigorous mixing was continued. The pH of the solution was subsequently lowered to about 4, and the precipitate obtained as a result of the lowering of the pH was collected by centrifugation at 2000 G for 10 minutes. The precipitate containing hydrolyzed wheat protein was washed twice by removing the supernatant, mixing with an equivalent volume of distilled water, and centrifuging the solution obtained at 2000 G for 10 minutes. The final precipitate of hydrolyzed wheat protein was dissolved in a volume of 1 to 5 mls distilled water per gram of wheat protein and the solution was equilibrated to pH 7. The neutral solution was lyophilized to obtain a dry powder.

To covalently crosslink PEG-NPC₂ with the hydrolyzed wheat protein, the hydrolyzed wheat protein was dissolved to a concentration of about 8% to about 12% (w/v) in an aqueous solution containing a strong inorganic base (e.g., NaOH, KOH, LiOH, RbOH and CsOH) or an organic base (e.g., triethylamine). This solution was combined with an aqueous solution of PEG-NPC₂ having a concentration ranging from about 13% to about 15% (w/v), which could be prepared by any of the methods described in Examples 1 to 3. The resulting solution was vigorously mixed until homogenization occurred.

Diluting the protein solution with a NaOH solution having an ionic strength that increased from about 0.19 N to about 0.24 N was found to decrease the gellification time from more than 4 minutes to less than 2 minutes.

The mixture was placed between two pieces of glass to form gel samples with a thickness of 1.45 mm. The resulting hydrogels were washed in EDTA/NaCl buffer to remove residual pNP and unreacted PEG and wheat protein.

It was observed that the hydrogels prepared by this method were mechanically strong and showed good elasticity.

### EXAMPLE 9

### Hydrogels with Antimicrobial Properties

To impart antimicrobial properties to the hydrogels, a loading solution containing an antimicrobial agent was integrated into the hydrogels. Specifically, hydrogels were prepared according to the methods described in Examples 4-8, then dehydrated and soaked in a solution containing NaCl (0.9 wt. %), EDTA (0.2 wt. %), NaH2PO4 (0.16 wt. %), and LIQUID GERMALL® PLUS (0.5 wt. %).

The antimicrobial properties of this formulation and others were evaluated in Examples 13 and 14 below.

### EXAMPLE 10

### Hydrogels Loaded with Active Ingredients

Medical articles of the invention may be prepared by integrating the hydrogels described in Examples 4-8 with active ingredient(s) as follows. The active ingredient(s) may be prepared as an aqueous solution or a solution in a different solvent. Hydrogels prepared according to the methods described in Examples 4-8 may then be dehydrated and soaked in the solution so prepared. An exemplary solution contains EDTA (0.2 wt. %), NaH2PO4 (0.16 wt. %), and caffeine (2 wt. %) in water.

### EXAMPLE 11

### Evaluation of the Degree of Swelling of Hydrogels

A series of studies were performed to evaluate the degree of swelling of certain hydrogel embodiments that may be included in the medical articles of the invention. Specifically, buffer solutions with various ionic strengths and pH values were used to swell the hydrogels. Weight differences in the hydrogels before and after swelling were measured to evaluate how ionic strength and pH influence the water content and the volume of the hydrogels.

A. Water Content and Water Uptake Versus Ionic Strength

To determine the effect of ionic strength on the water content and water uptake of the hydrogels, hydrogels prepared by the method described in Example 7 were poured between two plates of glass separated by 1-mm spacers. Hydrogels having a volume of 1.25 ml were subsequently allowed to swell and equilibrate in a solution of 10 mM NaCl to the point where no pNP was detectable by absorbency readings at 400 nm.

Subsequently, the same hydrogels were allowed to equilibrate in different concentrations of phosphate buffer at pH 6 by washing five times for one hour each time in 40 ml of buffer. The different concentrations of phosphate buffer used were the following: 100 mM, 75 mM, 50 mM, 25 mM, 12.5 mM, 10 mM, 5 mM, 1 mM, 0.1 mM and 0 mM.

For each concentration of buffer, the hydrogels were removed from solution, the water on their surfaces was blotted and the hydrogels, then in their swollen state (Wₛ), were weighed. The hydrogels were later dried to a constant weight in an oven at 80° C. and this dry weight (W₀) was measured. The results were then used to calculate the water content (C_{w}) and water uptake (Cᵤ) in accordance with equations (1) and (2) (R. J. LaPorte, Hydrophilic Polymer Coatings for Medical Devices: Structure/Properties, Development, Manufacture and Applications 41-44 (Technomic Publishing Company 1997)), below:
C_{w}=[(Wₛ−W₀)/Wₛ]×100 (1)
Cᵤ=[(Wₛ−W₀)/W₀]×100 (2)
Results

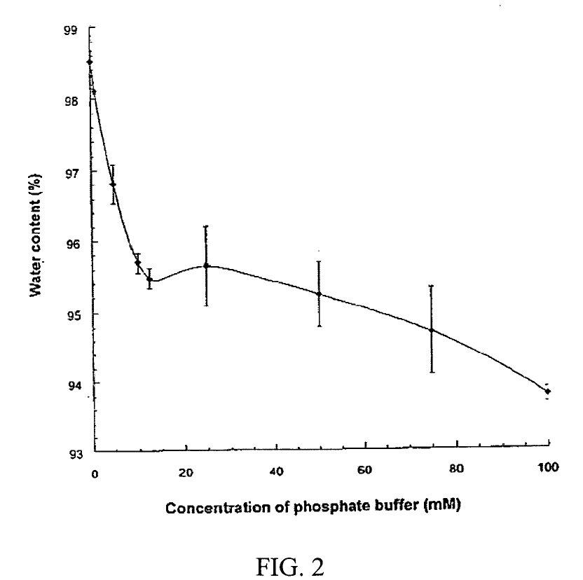

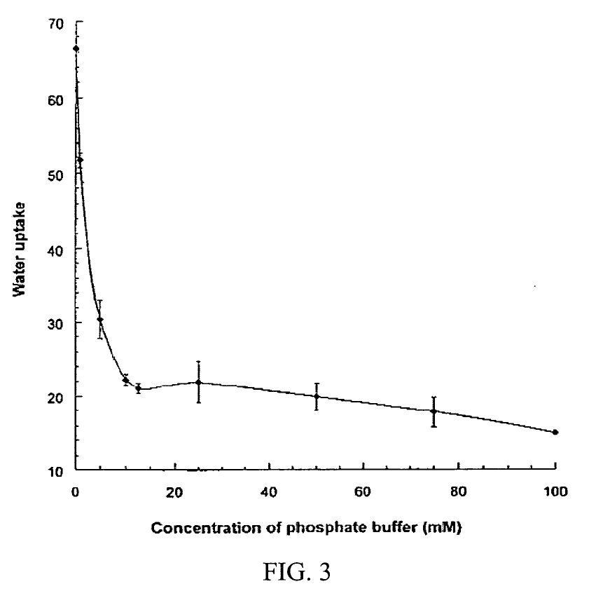

The effect of the ionic strength of the buffer solutions on the water content and water uptake of the hydrogels is shown graphically in FIGS. 2 and 3, respectively. It was observed that the water content (C_{w}) did not differ significantly from about 95% when the buffer concentration was in the range between 10 mM and 100 mM. This is even more apparent when the same results are presented in terms of water uptake (Cᵤ). As shown in FIG. 3, the water uptake was fairly constant with a value of around 20 times the dry weight of the hydrogel when the buffer concentration was in the range between 10 mM and 100 mM. There is, however, an increase in swelling when buffer concentrations of lower than 10 mM were used, reaching a maximum when deionized water was used. In the absence of ionic strength, it is expected from these data that the swelling of the hydrogel can attain a water content (C_{w}) of about 99%, corresponding to a water uptake (Cᵤ) of about 70 times the dry weight of the hydrogel.

B. Water Content and Water Uptake Versus pH

Using the procedures described in Part A, hydrogels were allowed to equilibrate in 10 mM phosphate buffer solution or 10 mM borate buffer solution having different pHs by washing five times for one hour each time in 40 ml of these buffers. Phosphate buffer solutions having pH values of 4, 6 and 7 were used. Borate buffer solutions having pH values of 9 and 11 were used.

Dry weights of the hydrogels (W₀) and their weights in the swollen state (Ws) were measured as described in Part A, and the results were used to calculate the water content (C_{w}) and water uptake (Cᵤ) in accordance with equations (1) and (2) above.

Results

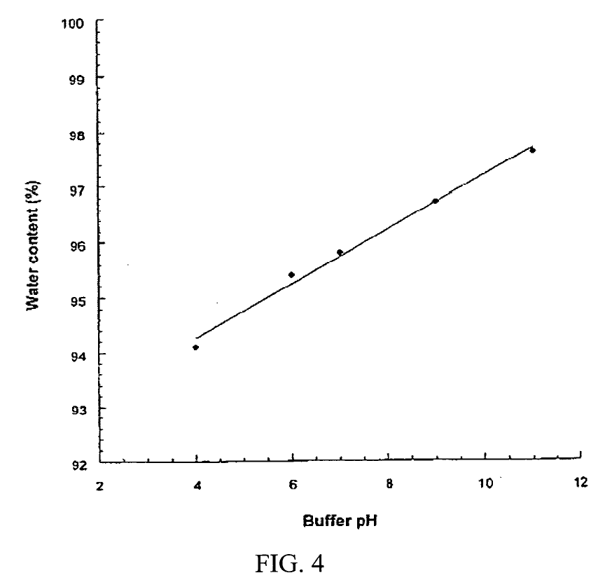

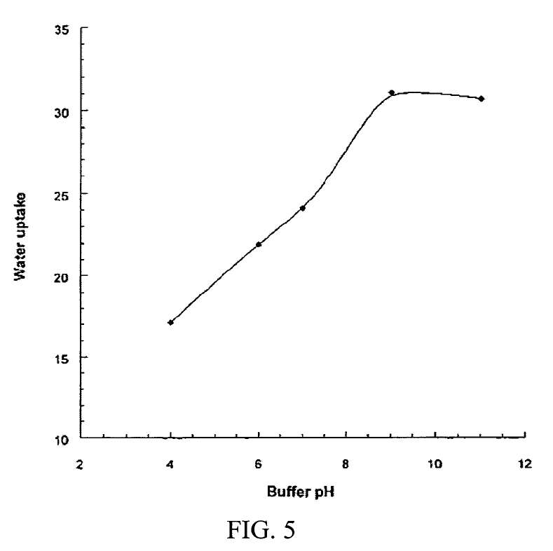

The effect of the pH of the buffer solutions on the water content and water uptake of the hydrogels is shown graphically in FIGS. 4 and 5, respectively. It was observed that the water content (C_{w}) was directly proportional to the pH of the solution, increasing from about 94% to about 97.5% as the pH increased from 4 to 11. The same trend was observed when the water uptake (Cᵤ) was considered. It can be seen from FIG. 5 that the water uptake was directly proportional to the pH of the solution, ranging from about 17 times the dry weight to about 30 times the dry weight as the pH increased from 4 to 11. Without being bound by any particular theory, it is believed that these variations in water content (C_{w}) and water uptake (Cᵤ) can be attributed to the low solubility of the hydrolyzed soy protein comprising the hydrogel at low pH and its increased solubility at high pH.

C. Volume of Hydrogels Versus Ionic Strength

To determine the effect of ionic strength on the volume of the hydrogels, hydrogels prepared by the method described in Example 7 were poured between two plates of glass separated by 1-mm spacers. Hydrogels having a volume of 1.25 ml were initially weighed just after synthesis to measure their volumes in their unexpanded state. Subsequently, the hydrogels were allowed to equilibrate in different concentrations of phosphate buffer at pH 6 by washing five times for one hour each time in 40 mls of buffer. The different concentrations of phosphate buffer used were the following: 100 mM, 75 mM, 50 mM, 25 mM, 12.5 mM, 10 mM, 5 mM, 1 mM, 0.1 mM and 0 mM.

For each concentration of buffer, the hydrogels were removed from solution, the water on their surfaces was blotted and the hydrogels, then in their expanded state, were weighed. The volume increase in the expanded hydrogels was calculated by dividing the weight of the hydrogel in its expanded state by the weight of the hydrogel in its unexpanded state.

Results

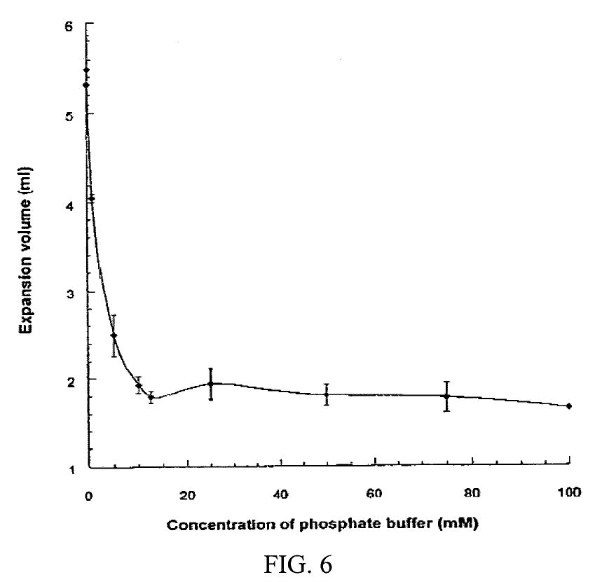

The effect of the ionic strength of the buffer solutions on the volumes of the hydrogels is shown graphically in FIG. 6. It was observed that the volume of the expanded hydrogels did not differ significantly from about 1.8 times the volume of the unexpanded hydrogels when the buffer concentration was in the range of between 10 mM and 100 mM. There was, however, an increase in volume when buffer concentrations lower than 10 mM were used, reaching a maximum when deionized water was used. In the absence of ionic strength, it was found that the hydrogels could expand to about 5.5 times of their volume in the unexpanded state.

D. Volume of Hydrogels Versus pH

Using the procedures described in Part C, hydrogels were allowed to equilibrate in 10 mM phosphate buffer solution or 10 mM borate buffer solution having different pHs by washing five times for one hour each time in 40 ml of these buffers. Phosphate buffer solutions having pH values of 4, 6 and 7 were used. Borate buffer solutions having pH values of 9 and 11 were used. The volume increase in the expanded hydrogels was calculated as described in Part C.

Results

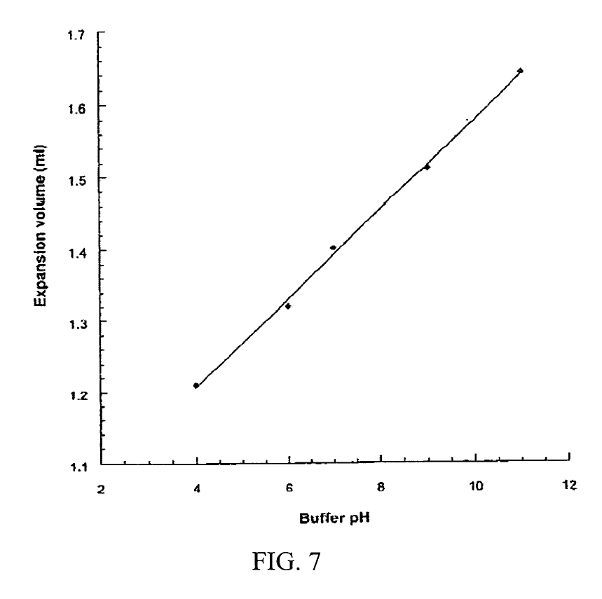

The effect of the pH of the buffer solutions on the volumes of the hydrogels is shown graphically in FIG. 7. It was observed that the volume of the expanded hydrogels was directly proportional to the pH of the solution, increasing from about 1.2 times the unexpanded volume of the hydrogel to about 1.65 times the unexpanded volume of the hydrogel as the pH increased from 4 to 11. Without being bound by any particular theory, it is believed that these variations in volumes can be attributed to the low solubility of the hydrolyzed soy protein comprising the hydrogel at low pH and its increased solubility at high pH.

The four studies together demonstrated that the hydrogels of the invention are highly absorbent and are capable of containing up to 99% by weight of water, which is equivalent to 70 times their dry weight.

### EXAMPLE 12

### Cytotoxicity Study

The biocompatibility of hydrogels was assessed in vitro by measuring their cellular toxicity using two different assays: MTT and neutral red uptake.

The in vitro tetrazolium-based colorimetric assay (MTT) formation, first described by Mosmann (Mosmann, T. (1983) J. IMMUNOLOGICAL METHODS 65: 55-63) to detect mammalian cell survival and proliferation, is a rapid calorimetric method based on the cleavage of a yellow tetrazolium salt 3-(4,5-dimethyl-thiazol-2,5-diphenyl-tetrazolium bromide) to purple formazan crystals by mitochondrial deshydrogenase enzymes of metabolically active cells. This conversion requires an intact mitochondrial system and depends on the level of metabolic activity of the cells. Since the amount of formazan generated can be quantified and is directly proportional to the number of viable (but not dead) cells, this method can be used to measure with precision cell survival and cell proliferation.

Neutral red is a lysosomal-specific probe used for assessing cytotoxicity (Borenfreund et al. (1984) J. TISSUE CULTURE METHODS 9: 83-92). This assay measures the growth rate of a population of cultured mammalian cells. Viable cells take up the neutral red dye and transport it to a specific cellular compartment, the lysosome. The uptake, transport, and storage of neutral red dye occurs via active biological processes that require energy, as well as intact cellular and lysosomal membranes. Damage to any of the systems involved in the process (or a reduction in cell number due to cell death), would result in decreased uptake of the neutral red dye in a given number of cells. Neutral Red uptake assay is undergoing validation as an in vitro alternative to the Draize test in a number of internationally validation programs such as those organized by the Commission of the European Communities (CEC); the Cosmetics, Toiletries and Fragrance Association (CTFA), and Soaps and Detergent Association (SDA) of the United States.

The cell cultures used in the MTT and neutral red uptake tests were human keratinocytes and fibroblasts isolated from the skin of a 22-year-old man (Germain et al. (1993) BURNS 19: 99-104; Rompré et al. (1990) IN VITRO CELLULAR AND DEVELOPMENTAL BIOLOGY-ANIMAL 26: 983-99). Briefly, the biopsy fragments were first treated with thermolysine (500 μg/ml) in Hepes buffer containing Ca²⁺ overnight at 4° C., before being separated from dermis with forceps. Epidermis was then treated with trypsin (0.05%) and EDTA (0.1%) in PBS buffer to release individual cells.

Isolated fibroblasts were plated at the density of 1.6×10⁴ into 12-well plates and grown in 1 ml of DMEM medium containing 10% fetal calf serum, 100 U/ml penicillin and 25 μg/ml gentamycin. Isolated keratinocytes from the same donor were plated into 12-well plates at the density of 2×10⁴ in the presence of 16×10⁴ irradiated mouse 3T3 fibroblasts, and grown in 1 ml of DMEM/Hams F12 (3/1; v/v) supplemented with 10 μg/ml EGF, 5 μg/ml bovine insulin, 5 μg/ml human transferrine, 2×10⁻⁹ M triiodo-L-thyronine, 10⁻¹⁰ M cholera toxin, 0.4 μg/ml hydrocortisone and 5% fetal calf serum. All the cultures were undertaken at 37° C. and 8% CO₂.

Hydrogel samples used in these studies were prepared as described in Example 7 (PEG-soy hydrogels). Prior to use, the PEG-soy hydrogels were dehydrated successively in 50/50, 60/40 and 70/30 ethanol/water (v/v) solutions, then rehydrated twice in phosphate buffered saline solution for 1 hour at room temperature under gentle agitation. The hydrogels were cut into round pieces fitting into 12-well culture plates, then soaked overnight in the adequate culture medium at 37° C. The culture medium was refreshed 1 hour before use.

After 48 hours at 37° C., 8% CO₂, the culture medium was removed from the cell cultures and one PEG-soy hydrogel (soaked in the appropriate culture medium as described above) was applied onto the cell cultures in the presence of 100 μl of the corresponding medium (in order to avoid the complete dehydration of the cells). Addition of 1 ml appropriate culture medium, without PEG-soy hydrogel, to the cells represented the control.

The PEG-soy hydrogel and culture media were renewed every day for 3 days (Day 3 to Day 5). Photographs were taken for each culture condition at Day 2 and Day 6 using a Nikon Eclipse TS 100 microscope (4×) with Nikon E995 camera. Experiments were carried out in triplicate for each culture condition and cell line.

A. MTT-Test

At Day 6, PEG-soy hydrogels were removed from the cell cultures and the cells were washed twice with phosphate-buffered saline. 1 ml of a 1 mg/ml MTT solution in PBS was added to each well and allowed to incubate for 3 hours at 37° C. and 8% CO₂. When the MTT incubation was complete, the unreacted dye was removed by aspiration. To each well, 0.8 ml acidified isopropyl alcohol (25 mM HCl in isopropanol) was added to solubilize the blue formazan crystals. Complete solubilization of the dye was achieved by shaking the plate vigorously. 100 μl of each sample was transferred in triplicate to a 96-well microplate. The optical density (OD) of each well was then measured with a microplate spectrophotometer (Biochrom Ultrospec 3000 UV/Visible spectrophotometer) at 540 nm. The spectrophotometer was calibrated to zero absorbance using wells that only contained MTT.

B. Neutral Red

At Day 6, the PEG-soy hydrogels were removed from the cell cultures and the cells were washed 2 times with phosphate-buffered saline. 1 ml of a 50 μg/ml neutral red solution in DMEM medium was added to each well and allowed to incubate for 3 hours at 37° C. and 8% CO₂. When the incubation was complete, the unreacted dye was removed by aspiration, and the cells were washed 2 times with PBS. 0.4 ml acetic acid/ethanol/water (1/50/49; v/v/v; lysis buffer) was added to each well and mixed thoroughly to ensure complete lysis of the cells. 100 μl of each sample was transferred in triplicate to a 96-well microplate and was then diluted 2 times with lysis buffer. The optical density (OD) of each well was then measured with a microplate spectrophotometer (Biochrom Ultrospec 3000 UV/Visible spectrophotometer) at 540 nm. The spectrophotometer was calibrated to zero absorbance using wells that had only contained lysis buffer.

The absorbance of the untreated control was defined as 100% viability. Statistical analyses were performed using Excel software by non-parametric Student-Newman-Keuls test.

C. Results

It was observed that the morphologies of neither the fibroblast culture nor the keratinocyte culture were affected after 4 days of contact with the PEG-soy hydrogels. Cell growth did appear to slow down in the presence of the hydrogels, but this could be because both keratinocyte and fibroblast cultures were less confluent in the presence of the PEG-soy hydrogels as compared to the untreated control.

Absorbance data measured for the different cell cultures are presented in Table 3 below and are expressed as the percent of cellular viability relative to untreated controls, i.e., cells grown in the absence of PEG-soy hydrogels.

As indicated in Table 3, a significant decrease in the percentage of viable fibroblasts and keratinocytes was observed in the MTT test when the cells were cultured in the presence of PEG-soy hydrogels as compared with the control. On the other hand, the neutral red uptake test indicated no significant difference between control and PEG-soy hydrogels cultures with respect to cellular viability for both keratinocytes and fibroblasts. Taken together, these results strongly suggest that the decrease observed in the metabolic activity of keratinocytes and fibroblasts was not due to a toxic effect of the PEG-soy hydrogels themselves, but to the fact that the cell cultures were less confluent in the presence of the PEG-soy hydrogels. As such, it was concluded that the absence of PEG-soy hydrogels-induced cytotoxicity on human keratinocyte and fibroblast cultures demonstrated that the PEG-soy hydrogels prepared according to the method described in Example 7 are non-toxic and biocompatible.

TABLE 3
Cellular viability estimated by MTT and neutral red cytotoxicity
assays using keratinocyte and fibroblast monocultures following
4 days of contact with PEG-soy hydrogels.

| | Fibroblasts | | Keratinocytes | |
|---|---|---|---|---|
| | Control | Hydrogel | Control | Hydrogel |
| | Viability (%) | Viability (%) | Viability (%) | Viability (%) |
| MTT | 100 ± 6 | 73 ± 2 | 100 ± 14 | 74 ± 3 |
| Neutral red | 100 ± 6 | 90 ± 8 | 100 ± 4 | 99 ± 7 |

### EXAMPLE 13

### Human Tolerance Tests

In vivo studies involving acute primary irritation and cumulative irritation tests were performed on human healthy volunteers. The studies demonstrated the biocompatibility of PEG-soy hydrogels on human skin.

A. Evaluation of Acute Primary Tolerance

To assess tolerance of the hydrogels of the invention on human skin, 61 male and female subjects were enrolled in the study after verification of inclusion and exclusion criteria. Subjects fulfilled specific inclusion criteria including not being pregnant or breastfeeding, being over 18 years old, having healthy skin, and not having used any dermatological or cosmetic preparation on the test area within 5 days before the beginning of the study. The study was conducted in accordance with the ICH Harmonized Tripartite Guidelines for Good Clinical Practice (ICH Guidance for Industry: E6 Good Clinical Practice Consolidated Guidance (1996)).

Briefly, four test sites were designated and located on the outer aspect of the upper arm of each subject. Test products were randomly applied on either arm for four hours under occlusion by means of Hayes Epicutantest Chambers and in a balanced Latin square design. Hayes Epicutantest Chambers are square plastic test chambers (1 cm×1 cm) provided with an integrated piece of filter paper designed for occlusive patch testing. The formulations of the products tested are shown below in Table 4.

TABLE 4
Formulations of test products.

| Test Product | Ingredients |
|---|---|
| PEG-Soy Hydrogel | Water, PEG, hydrolyzed soy proteins, |
| | EDTA, NaCl, sodium phosphate monobasic, |
| | diazolidinyl urea, iodopropynyl |
| | butylcarbamate, and propylene glycol. |
| 2^{nd} Skin ® Moist | Not available. |
| Burn Pads | |
| Positive Control | 0.5% aqueous solution of sodium |
| | lauryl sulphate |

The hydrogels used in this test were prepared as described in Example 7, then soaked in a solution containing 0.9% NaCl, 0.5% LIQUID GERMALL® PLUS (International Specialty Products, Wayne, N.J.), 0.2% EDTA, and 0.16% sodium phosphate monobasic. The final pH of the hydrogels was adjusted to about 5.5.

The tolerance of the hydrogels was tested against a positive control and a negative control and further compared with the tolerance of a commercially available hydrogel product, namely 2nd SKIN® Moist Burn Pads (MBP) from Spenco Medical Corp. (Waco, Tex.). The positive control was prepared by pipetting 40 μl of a 0.5% aqueous solution of sodium lauryl sulphate (SLS) into the Hayes Epicutantest Chambers, whereas an empty Hayes Epicutantest Chambers served as the negative control.

Visual assessments of the test sites were conducted by trained personnel on day 1 (D1) prior to application of the test products and 5 minutes, 30 minutes, and 60 minutes after patch removal, on day 2 (D2) (i.e., after 24 hours of application), and on day 4 (D4) (i.e., after 72 hours of application). Possible skin reactions to the products were scored on a scale that describes the amount of erythema, edema and other features indicative of irritation (according to The Scoring Scale proposed by the U.S. Food and Drug Administration (FDA) for the evaluation of skin irritancy and sensitization potential (FDA Guidance for Industry: Skin Irritation and Sensitization Testing of Generic Transdermal Drug Products—Appendix A; CDER December 1999)). The scoring scale is reproduced below in Table 5.

TABLE 5
Visual evaluation of skin tolerance.

| Grade | Dermal response | Notation | Other effects |
|---|---|---|---|
| 0 | No evidence of irritation | X | No other changes |
| 1 | Minimal erythema, barely perceptible | A | Slight glazed appearance |
| 2 | Definite erythema, readily visible; | B | Marked Glazing |
| | minimal edema or minimal papular | | |
| 3 | Erythema and papules | C | Glazing with peeling & cracking |
| 4 | Definite erythema | D | Glazing with fissures |
| 5 | Erythema, edema, and papules | E | Film of dried serous exudate covering |
| | | | all or part of the patch site |
| 6 | Vesicular eruption | F | Small petechial erosions and/or scabs |
| 7 | Strong reaction spreading beyond test site | | |

The scores obtained with regard to any dermal reactions observed in the 61 subjects over the four-day test period were added, thus giving one single irritancy sum score for each test product (presented in the first row of Table 6 below). Table 6 further includes data regarding the specific number of subjects that have shown any dermal reactions (in the second row), the minimum and maximum irritancy score that has been assigned to any of the 61 subjects on any given day during the test period (third and fourth rows), and the minimum and maximum sum score that has been assigned to any subject over the 4-day period (the fifth and sixth rows).

Simultaneously, clinical observations and any reaction reported by the test subject were recorded. The types of reactions observed and reported on days 1, 2, and 4 (D1, D2, and D4) are summarized in Table 7. The numbers in each column represent the number of subjects that have shown or experienced the dermal reaction listed with regard to each of the test product.

Results

As indicated in Table 6, no dermal reaction in visual scoring was shown on untreated occluded control area (negative control). Moreover, as shown in Table 7, few clinical observations were made on these test sites. Slight glazed appearance was observed in three subjects, and marked glazing was observed in one subject. A fourth subject experienced dryness, and a fifth subject reported slight itch. Overall, a total of six observations were made indicating that approximately 10% (6/61) of clinical observations resulted from the application of the test patch itself.

On sites treated with SLS, numerous reactions were recorded in 19 subjects, all of which experienced Grade 1 reactions (minimal erythema). In one subject, the reaction lasted during the entire study period (i.e., having an irritancy sum score of 3). In two others, it lasted 2 days (i.e., having an irritancy sum score of 2). Otherwise, the reactions were short-lived and disappeared by day 2. These observations are consistent with other tests that were conducted to evaluate skin reactions caused by a short-term application of a low concentration of SLS (see, e.g., Tupker et al. (1997) CONTACT DERMATITIS 37: 53-69, and Gloor et al (2004) SKIN RES. TECHNOL. 10: 114-148) and demonstrates that the group of volunteers was suited to detect even a low irritation potential. Clinically, a total of 15 observations were noted on day 1, 32 on day 2, and 22 on day 4. Most of the observations were “slight glazed appearance” and “marked glazing,” although one subject did report dryness on day 2. These results confirm that SLS is a suitable positive control.

There were almost no reactions on removal of the tested hydrogel patches after four hours of application. Only 3 reactions in 3 volunteers were scored Grade 1 on Day 1 and no others on the following days. Clinically, almost the same observations were made on the areas treated with the tested hydrogels compared to the areas treated with the empty patches (negative control). The same subject in both treatment groups showed dryness and the same other subject reported slight itch. Overall, 7 observations were made in 61 total subjects for a clinical observation rate of about 11%. These results (similar to those of the negative control) lead to the conclusion that these clinical observations were due to the patches themselves and not to the tested hydrogels. Therefore, it was concluded that the tested hydogels were very well tolerated under the conditions of this test.

On removal of the test patches containing 2nd Skin® Moist Burn Pads (MBP) after four hours of application, mild skin reactions similar to those induced by the tested hydrogels were observed. Few clinical observations were made after treatment with MBP. No subject reported itch. Scaly skin was registered from Day 1 to Day 4 in one volunteer, which could be attributed to the dryness of the subject's skin in general. The observations otherwise were almost identical between the test sites for the tested hydrogels and the MBPs. Thus, no differences in tolerance were observed between the tested hydrogels and the reference product under the test conditions.

TABLE 6
Results of evaluation of skin tolerance - dermal reactions observed
in sixty-one subjects over a 4-day test period (the scoring scale
used corresponds to the one reproduced in Table 5).

| Sum of scores | Treatment group | | | |
|---|---|---|---|---|
| for dermal reactions | | Positive | Negative | |
| Parameter | Hydrogel | Control | Control | MBP |
| Sum of scores | 3.00 | 23.00 | 0 | 4.00 |
| N (reacting subjects) | 3 | 19 | 0 | 3 |
| Minimum single score | 0 | 0 | 0 | 0 |
| Maximum single score | 1 | 1 | 0 | 1 |
| Minimum sum | 0 | 0 | 0 | 0 |
| Maximum sum | 1 | 3 | 0 | 2 |

TABLE 7
Results of evaluation of skin tolerance - other effects observed
in sixty-one subjects over a 4-day test period (the scoring scale
used corresponds to the one reproduced in Table 5).

| | Treatment group | | | | | | | | | | | |
|---|---|---|---|---|---|---|---|---|---|---|---|---|
| Summary of clinical observations | | | | Positive | | | Negative | | | | | |
| Product | Hydrogel | | | Control | | | Control | | | MBP | | |
| PARAMETER | D1 | D2 | D4 | D1 | D2 | D4 | D1 | D2 | D4 | D1 | D2 | D4 |
| A: Slight glazed | 4 | 0 | 0 | 14 | 29 | 20 | 3 | 0 | 0 | 1 | 0 | 0 |
| appearance | | | | | | | | | | | | |
| B: Marked glazing | 1 | 0 | 0 | 1 | 3 | 2 | 1 | 0 | 0 | 0 | 0 | 0 |
| C: Glazing with peeling | 0 | 0 | 0 | 0 | 0 | 0 | 0 | 0 | 0 | 0 | 0 | 0 |
| & cracking | | | | | | | | | | | | |
| D: Glazing with fissures | 0 | 0 | 0 | 0 | 0 | 0 | 0 | 0 | 0 | 0 | 0 | 0 |
| E: Film of dried serious | 0 | 0 | 0 | 0 | 0 | 0 | 0 | 0 | 0 | 0 | 0 | 0 |
| exudate covering all | | | | | | | | | | | | |
| or part of the patch | | | | | | | | | | | | |
| site | | | | | | | | | | | | |
| F: Small petechial | 0 | 0 | 0 | 0 | 0 | 0 | 0 | 0 | 0 | 0 | 0 | 0 |
| erosions and/or | | | | | | | | | | | | |
| scabs | | | | | | | | | | | | |
| Other symptoms | | | | | | | | | | | | |
| Dryness | 1 | 0 | 0 | 0 | 1 | 0 | 1 | 0 | 0 | 1 | 0 | 0 |
| Slight itch | 1 | 0 | 0 | 0 | 0 | 0 | 1 | 0 | 0 | 1 | 0 | 0 |
| Scaly skin | 0 | 0 | 0 | 0 | 0 | 0 | 0 | 0 | 0 | 1 | 1 | 1 |
| Total observations | 7 | 0 | 0 | 15 | 32 | 22 | 6 | 0 | 0 | 3 | 1 | 1 |

B. Evaluation of Cumulative Irritancy and Sensitization Potential

To evaluate the cumulative irritancy and sensitization potential of the hydrogels, 107 male and female subjects were enrolled in a Human Repeated Insult Patch test (HRIPT) after verification of inclusion and exclusion criteria. Subjects fulfilled specific inclusion criteria including not being pregnant or breastfeeding, being over 18 years old, having healthy skin, and not having used any dermatological or cosmetic preparation on the test area within 5 days before the beginning of the study. The methodology used was an adaptation from that described in Marzulli et al. (1976) CONTACT DERMATITIs 2:1-17.

Briefly, the tested hydrogels were applied under occlusion on the outer aspect of the upper arm for a defined time. The applications were repeated 9 times over a period of 3 consecutive weeks, a duration necessary for the possible induction of an immune response. The irritancy potential was evaluated and compared to the irritancy potential of the standard, SLS. After a two-week rest period with no treatment, the tested hydrogels were applied under occlusion to the induction site and to a virgin site on the volar side of the underarm for a defined period of time to trigger a possible immune response.

The hydrogels used in this test were prepared as described in Example 7, then soaked in a solution containing 0.9% NaCl, 0.5% LIQUID GERMALL® PLUS (International Specialty Products, Wayne, N.J.), 0.2% EDTA, and 0.16% sodium phosphate monobasic. The final pH of the hydrogels was adjusted to about 5.5. A 0.01% aqueous solution of SLS served as the positive control, while injectable-grade water served as the negative control.

During the induction phase, visual assessments of the test sites were conducted by trained personnel prior to application of the test products, after 48 hours of contact on Days 3, 5, 10, 12, 17, and 19, and after 72 hours of contact on Days 8, 15, and 22. Possible skin reactions to the products were scored according to the scale reproduced in Table 5 above. The total score was calculated by summing each individual's score over the 22-day test period.

In the challenge phase, visual assessments of the test sites were conducted prior to application of the test products on Day 36 and 30 minutes after patch removal on Days 38, 39, and 40 (i.e., after 48, 72, and 96 hours of contact, respectively). The sensitization potential was classified as shown in Table 8 below. The grades referred to in Table 8 correspond to the scoring scale provided in Table 5 above. In summary, the test product is considered to have a low sensitization potential if none of the subjects reported a grade 2 or higher dermal response on days 38 to 40 and no more than two subjects reported a grade 1 dermal response on days 38 to 40. A moderate sensitization potential is assigned if a maximum of 2 subjects reported a grade 2 or higher dermal response on days 38 to 40 and a maximum of 4 subjects reported a grade 1 response on days 38 to 40. A high sensitization potential is assigned if 3 or more subjects reported a grade 2 or higher dermal response on days 38 to 40 and 5 or more subjects reported a grade 1 response on days 38 to 40.

TABLE 8
Classification of sensitization potential.

| Number of subjects reacting with | Number of subjects reacting with | Category of sensitization |
|---|---|---|
| grade ≧2 on days 38 and 39 and 40 | grade 1 on days 38 and 39 and 40 | potential |
| None | Max. 2 | Low |
| Max. 2 | Max. 4 | Moderate |
| 3 or more | 5 or more | High |

The observations made for both the hydrogels and the controls are summarized in Tables 9 and 10 below. Specifically, Table 9 summarizes the number and type of observations made during the induction phase with regard to each of the test product. The cumulative irritancy score represents the sum of the irritancy scores assigned on days 3, 5, 8, 10, 12, 15, 17, 19, and 22. As it is well-known that SLS has a high sensitization potential, testing with SLS was not continued beyond the induction phase. Table 10 summarizes the number and type of observations made during the challenge phase associated with the application of the hydrogel and the negative control only. An irritancy score was assigned to each induction and virgin site on days 36, 38, 39 and 40, and their respective scores were added up separately to produce the cumulative irritancy score presented in the fourth column of Table 10. The fifth and sixth columns indicate the number of subjects that experienced a grade 2 or greater response on each of days 38, 39 and 40, and the number of subjects that experienced a grade 1 response on each of days 38, 39, and 40.

Results

TABLE 9
Cumulative irritancy test results with the application
of hydrogel over the 22-day induction phase.

| | | Number of | Cumulative |
|---|---|---|---|
| | Type of | reacting | Irritancy |
| Induction Phase | Reactivity | subjects | Score |
| Hydrogel | Minimal erythema | 5 | 6 |
| Positive Control (SLS) | Minimal erythema | 11 | 21 |
| Negative Control (water) | Minimal erythema | 3 | 5 |

TABLE 10
Cumulative irritancy test results with the application
of hydrogel during the challenge phase.

| | Type of | Number of | Cumulative | >Grade 2 | Grade 1 |
|---|---|---|---|---|---|
| Challenge Phase | Reactivity | reacting subjects | Irritancy Score | response | response |
| Hydrogel (induction | Minimal | 1 | 1 | 0 | 1 |
| site) | erythema | | | | |
| Hydrogel (virgin | Minimal | 1 | 1 | 0 | 1 |
| site) | erythema | | | | |
| Negative Control | No evidence of | 0 | 0 | 0 | 0 |
| (induction site) | irritation | | | | |
| Negative Control | No evidence of | 0 | 0 | 0 | 0 |
| (virgin site) | irritation | | | | |

As shown in Table 9, during the induction phase, no significant irritation reaction was observed on the sites where hydrogels had been applied. Only 5 volunteers exhibited a transient minimal erythema, which was barely perceptible. The cumulative irritancy score for the tested hydrogels was 6. Clinically, 2 subjects exhibited slight glazed appearance, but these observations only appeared for one day in each of the 2 subjects.

No significant irritation reaction was observed on the negative control sites. Three volunteers exhibited a transient minimal erythema, which was barely perceptible. The cumulative irritancy score was 5. Clinically, 13 subjects exhibited slight glazed appearance. Two of these subjects also exhibited marked glazing, and/or glazing with peeling and cracking on at least one occasion. Most of these observations were temporary, except for the two subjects who reported marked glazing and four other subjects who also exhibited prolonged reaction to the negative (water) control.

By comparison, the cumulative irritancy score for the positive control standard, SLS aqueous solution, was 21. In addition, a slight glazed appearance and/or marked glazing were observed on the positive control sites in 20 subjects. These symptoms often appeared for multiple days. Among these 20 subjects, seven exhibited these symptoms for at least four of the days that evaluations were undertaken.

As shown in Table 10, during the challenge phase, only 1 person reported minimal erythema (a Grade 1 reaction) on both the induction site and on the virgin site when the hydrogels were applied. According to the classification method provided in Table 8, the tested hydrogels therefore are considered to have a low sensitization potential. No sign of irritation was observed when the negative control (i.e., water) was applied on either the induction site or the virgin site.

Therefore, under the experimental conditions adopted, the repeated applications of certain hydrogel-containing medical articles of the invention under occlusion on a panel of 107 volunteers induced no relevant reaction of irritation nor allergic reaction. The product was demonstrated to have good skin compatibility and can be classified as a low sensitization potential product.

Additionally, as demonstrated by the results obtained in these two studies, the absence of erythema and edema induced by the unique and repeated applications of the hydrogels confirmed their biocompatibility on human skin.

### EXAMPLE 14

### Hydrating Effect of Hydrogels

Optimal hydration level of the skin can be important for many physiological functions including barrier function and thermoregulation. Water ensures softness and flexibility of tissues. When the level of hydration is low, skin becomes rough, dry, and inflexible with the tendency of rupture on applied stress. Skin hydration depends on the water-holding capacities of the stratum corneum. The stratum corneum is a dielectric corpus, and all changes in its hydration status are reflected by changes in the electric properties of the skin (e.g., its capacitance).

To study the hydrating effect of hydrogels that may be suitable for use with the medical articles of the invention, two studies were conducted. In the first study, the short-term hydrating effect of tested hydrogels were evaluated against a positive control, a negative control, and a commercially available hydrogel product. In the second study, the long-term hydrating effect of tested hydrogels were evaluated against a positive control, a negative control, and an unoccluded site.

A. Short-Term Hydrating Effect

During the acute primary tolerance test described in Example 13, skin hydration measurements were taken on the same group of subjects with a Corneometer® CM825/MPA 8 device (Courage and Khazaka, Germany) equipped with a 49 mm² probe. The probe was gently pressed against the skin at a pressure of 3.56 N, and the capacitance of the skin was recorded. To account for the variation of hydration level at different sites of the skin, the application of the test product was randomized, and three consecutive measurements were taken on each skin area for each volunteer as described in Berardesca (1997) SKIN RES. TECHNOL. 3: 126-132. All measurements were conducted under controlled conditions (temperature=22° C.±1° C.; relative humidity=50%±5%) after an acclimatization period of at least 30 minutes.

The data summarized in Table 11 were obtained prior to the application of the four test products and controls (T₀) as described in Example 13, as well as immediately after, 30 minutes after, 60 minutes after, and 24 hours after a four-hour application of the test products and controls (Tₙ=T₁ₘᵢₙ, T₃₀ₘᵢₙ, T₆₀ₘᵢₙ, T₂₄ₕᵣ). Capacitance as measured with Corneometer® are expressed in arbitrary units. A greater positive difference between the capacitance measured at Tₙ and the capacitance measured at T₀ represents a greater hydrating effect.

Result

At the site where the negative control was applied (i.e., the empty cell), increased skin hydration was observed for a short period of time after the patch was removed. The level of skin hydration returned to close to the initial level after the patch was removed for 30 minutes and did not vary much thereafter.

At the positive control site (i.e., where SLS was applied), a strong hyperhydration was observed immediately after the patch was removed. The hyperhydration was followed by an apparent dryness. This time course of skin hydration is well known after treatment with SLS (e.g., Fluhr et al. (2004) SKIN RES. TECHNOL. 10: 141-143).

By comparison, it was observed that after four hours of application of the tested hydrogels, the skin hydration level was greater than that measured after application of the negative control. The data, therefore, suggested that the tested hydrogels were able to provide more moisture than a simple occlusion. Although hydration values rapidly decreased after the first five minutes, the hydration levels were still higher than the negative control values measured at 30 and 60 minutes. At 24 hours, no significant difference was observed between the sites where the tested hydrogels had been applied and the two control sites.

It was further observed that although the 2nd Skin® Moist Burn Pads (MBP) were able to produce a higher skin hydration level than the negative control within the first five minutes after the test patches were removed. The skin hydration level was similar to the negative control level and lower than the level obtained with the tested hydrogels 30 minutes after the patch was removed. Again, at 24 hours, no significant differences were observed between MBP and the two controls.

TABLE 11
Short-term hydrating effect as measured as
capacitance expressed in arbitrary units.

| | T₀ | T_{1 min} | T_{30 min} | T_{60 min} | T_{24 hr} |
|---|---|---|---|---|---|
| Hydrogel | 35.3 ± 8.3 | 55.8 ± 13.4 | 38.5 ± 8.5 | 37.1 ± 8.0 | 38.6 ± 8.0 |
| Positive Control | 35.0 ± 8.5 | 69.4 ± 18.0 | 30.5 ± 6.5 | 28.9 ± 6.6 | 35.5 ± 8.7 |
| Negative Control | 35.3 ± 8.1 | 49.2 ± 11.8 | 36.6 ± 7.7 | 36.4 ± 7.1 | 38.4 ± 7.7 |
| 2^{nd} Skin ® Moist | 34.8 ± 8.5 | 53.3 ± 12.3 | 35.9 ± 8.5 | 34.9 ± 8.7 | 38.0 ± 8.7 |
| Burn Pads | | | | | |

B. Long-Term Hydrating Effect

During the cumulative irritancy test described in Example 13, 55 of the 107 volunteers participated in a concurrent hydration study. Skin hydration measurements were taken from these 55 subjects days 1, 5, 8, and 22, measuring the skin hydrating effect of the tested products after 72 hours of application.

The measurements were taken with a Corneometer® CM825/MPA 8 device (Courage and Khazaka, Germany) equipped with a 49 mm² probe. The probe was gently pressed against the skin at a pressure of 3.56 N, and the capacitance of the skin was recorded. To account for the variation of hydration levels in the varying sites of the skin, test product application was randomized, and three consecutive measurements were taken on each skin area for each volunteer as described in Berardesca (1997) SKIN RES. TECHNOL. 3: 126-132. All measurements were conducted under controlled conditions (temperature=22° C.±1° C.; relative humidity=50%±5%) after an acclimatization period of at least 30 minutes.

In addition to the tested hydrogel and the positive control containing SLS, a negative control containing water was applied to a third test site. Skin hydration measurements were also taken on a fourth unoccluded site. The results are summarized in Table 12. The values in Table 12 represent the Corneometer® readings taken on days 1, 8, 15, and 22, and are expressed in arbitrary units. A greater positive difference between the capacitance measured on day 1 and the capacitance measured on a subsequent day represents a greater hydrating effect.

Results

It was observed that at the sites where the tested hydrogels were applied, skin hydration consistently increased over the first 22 days of the study. In contrast, all the other sites revealed a general decrease and, at most, a very slight increase on day 22 in epidermal hydration.

To test the significance of these results, the data were further analyzed using the ANOVA technique (Duncan, A. J., “Analysis of Variance,” Quality Control and Industrial Statistics (Irwin Publishers, Homewood, Ill., 1986)). These further analyses confirmed that the tested hydrogels were able to increase skin hydration compared to the controls (SLS and water).

TABLE 12
Long-term hydrating effect as measured as
capacitance expressed in arbitrary units.

| | Day 1 | Day 8 | Day 15 | Day 22 |
|---|---|---|---|---|
| Hydrogel | 43.2 | 46.1 | 46.3 | 50.6 |
| SLS | 42.7 | 37.5 | 37.2 | 45.3 |
| Water | 42.5 | 36.6 | 36.9 | 45.2 |
| Unoccluded | 39.6 | 35.3 | 36.5 | 41.0 |

The two hydration studies together indicate that the tested hydrogels have measurable hydrating effects with both short-term and long-term usage.

### EXAMPLE 15

### Sterility and Antimicrobial Activity of Hydrogels

Studies were performed to evaluate the sterility and antimicrobial properties of four formulations of hydrogels that may be used with the medical articles of the invention. Specifically, challenge tests were carried out using the microbes listed in Table 13 below.

TABLE 13
Microbes used in challenge test.

| Microbe | ATCC Number |
|---|---|
| Candida albicans (CAN)*** | 10231 |
| Aspergillus niger (AN)*** | 16404 |
| Staphylococcus aureus (SA)** | 6538 |
| Bacillus cereus (BC)** | 14579 |
| Escherichia coli (ECOLI)* | 8739 |
| Salmonella arizonae (SAZ)* | 13314 |
| Klebsiella pneumoniae (KP)* | 13883 |
| Enterobacter cloacae (ENC)* | 13047 |
| Pseudomonas aeruginosa (PSA)* | 9027 |

*gram-negative bacteria
**gram-positive bacteria
***fungi

The four formulations were prepared as follows. Hydrogels prepared by the method described in Example 7 were used as controls. Additionally, hydrogels were prepared by the method described in Example 7 and then further loaded with integration solutions 1, 2, and 3, to create Formulations 1, 2, and 3, respectively. The compositions of the integration solutions are described in Table 14 below.

TABLE 14
Composition of integration solutions (all
values are given in weight percent).

| Integration | | | | LIQUID |
|---|---|---|---|---|
| Solution | NaCl | EDTA | NaH₂PO₄ | GERMALL ® PLUS |
| 1 | 0.9 | 0.2 | 0.16 | 0 |
| 2 | 0.9 | 0.2 | 0.16 | 0.1 |
| 3 | 0.9 | 0.2 | 0.16 | 0.5 |

Each formulation was inoculated with a standardized inoculum of the challenge microbes. The samples were incubated and assayed at 1 hour, 24 hours, 48 hours, 7 days, 14 days, and 21 days. Plate-count procedures were followed to determine the number of colonies per gram (CFU/g). The results are presented in Table 15 below.

Results

Formulation 1 was effective in killing almost all of each culture of Candida albicans and Pseudomonas aeruginosa within 14 days. A greater than 2-log reduction was observed for Staphylococcus aureus, Enterobacter cloacae, Bacillus cereus, and Escherichia coli within 14 days. With the use of Formulation 1, there was also no increase from the initial calculated count for any of the bacteria, yeast, and molds on days 14 and 28.

Formulation 2 (with the addition of 0.1 wt. % of LIQUID GERMALL® PLUS) was able to attain a greater than 2-log reduction of the three remaining studied microbes (i.e., Aspergillus niger, Salmonella arizonae, and Klebsiella pneumoniae) by day 7. In fact, Formulation 2 was effective enough to kill almost all of each culture of Candida albicans, Aspergillus niger, Staphylococcus aureus, Klebsiella pneumoniae and Pseudomonas aeruginosa by day 7. Almost all of each culture of Escherichia coli, Salmonella arizonae, and Enterobacter cloacae was killed by day 14. Although a significant number of Bacillus cereus were still present on day 21, Formulation 2 did achieve a greater than 3-log reduction within 21 days.

Formulation 3 (with the addition of 0.5 wt. % of LIQUID GERMALL® PLUS) was found to be especially effective, killing almost all of each culture of Candida albicans, Pseudomonas aeruginosa, Aspergillus niger, and Klebsiella pneumoniae within 24 hours, and Staphylococcus aureus, Escherichia coli, Salmonella arizonae, and Enterobacter cloacae within 48 hours. A greater than 5-log reduction with Bacillus cereus was also observed by the first 48 hours and that culture was almost entirely killed by Day 14.

TABLE 15
Antimicrobial properties of hydrogels of various formulations.

| | | Number of colonies per gram (CFU/g) | | | | | |
|---|---|---|---|---|---|---|---|
| Formulation | Microbe | 1 Hour | 24 Hours | 48 Hours | 7 Days | 14 Days | 21 Days |
| Control | CAN | 5.0 × 10⁵ | 2.6 × 10⁶ | 1.1 × 10⁶ | 1.9 × 10⁶ | >1.0 × 10⁶ | >1.0 × 10⁶ |
| Control | AN | 1.2 × 10⁵ | 3.6 × 10⁴ | 2.6 × 10³ | 6.0 × 10⁴ | 7.0 × 10⁴ | >1.0 × 10⁶ |
| Control | SA | 6.4 × 10⁷ | 1.2 × 10⁸ | 2.0 × 10⁸ | 2.1 × 10⁷ | >1.0 × 10⁶ | >1.0 × 10⁶ |
| Control | BC | 6.0 × 10⁶ | 1.6 × 10⁷ | 2.1 × 10⁶ | 1.9 × 10⁷ | >1.0 × 10⁶ | >1.0 × 10⁶ |
| Control | ECOLI | 4.4 × 10⁷ | 2.0 × 10⁸ | 2.6 × 10⁸ | 1.5 × 10⁸ | >1.0 × 10⁶ | >1.0 × 10⁶ |
| Control | SAZ | 3.2 × 10⁷ | 1.9 × 10⁸ | 4.9 × 10⁷ | 8.4 × 10⁷ | >1.0 × 10⁶ | >1.0 × 10⁶ |
| Control | KP | 2.4 × 10⁷ | 2.4 × 10⁸ | 1.5 × 10⁸ | 1.0 × 10⁸ | >1.0 × 10⁶ | >1.03 × 10⁶ |
| Control | ENC | 2.3 × 10⁷ | 1.6 × 10⁸ | 1.6 × 10⁸ | 1.3 × 10⁸ | >1.0 × 10⁶ | >1.0 × 10⁶ |
| Control | PSA | 8.8 × 10⁶ | 2.1 × 10⁸ | 1.3 × 10⁸ | 9.7 × 10⁷ | >1.0 × 10⁶ | >1.0 × 10⁶ |
| 1 | CAN | 1.1 × 10⁶ | 7.6 × 10⁵ | 8.6 × 10⁵ | 1.3 × 10⁵ | <10 | <10 |
| 1 | AN | 1.1 × 10⁵ | 8.6 × 10⁴ | 8.5 × 10⁴ | 8 × 10⁴ | 3.6 × 10⁴ | 3.9 × 10⁴ |
| 1 | SA | 3.1 × 10⁷ | 9.1 × 10⁶ | 1.1 × 10⁷ | 3.4 × 10⁵ | 8.0 × 10³ | 2.7 × 10² |
| 1 | BC | 2.8 × 10⁶ | 7.4 × 10⁴ | 3.2 × 10³ | 1.9 × 10³ | 1.8 × 10³ | 8.6 × 10² |
| 1 | ECOLI | 5.1 × 10⁷ | 1.3 × 10⁷ | 2.8 × 10⁷ | 3.2 × 10⁶ | 5.0 × 10⁵ | 8.0 × 10³ |
| 1 | SAZ | 3.4 × 10⁷ | 1.2 × 10⁷ | 1.5 × 10⁷ | 1.8 × 10⁷ | 1.3 × 10⁷ | 3.6 × 10² |
| 1 | KP | 1.9 × 10⁷ | 2.2 × 10⁶ | 6.7 × 10⁶ | 3.4 × 10⁶ | 2.0 × 10⁶ | 9.0 × 10³ |
| 1 | ENC | 1.8 × 10⁷ | 1.4 × 10⁷ | 8.2 × 10⁶ | 1.4 × 10⁶ | 8.4 × 10⁴ | 1.5 × 10² |
| 1 | PSA | 1.1 × 10⁷ | 2.3 × 10⁴ | 2.5 × 10⁴ | 80 | <10 | <10 |
| 2 | CAN | 1.3 × 10⁶ | 8.0 × 10⁵ | 5.6 × 10⁴ | <10 | <10 | <10 |
| 2 | AN | 1.2 × 10⁵ | 6.6 × 10⁴ | 2.4 × 10⁴ | <10 | 30 | <10 |
| 2 | SA | 2.7 × 10⁷ | 4.3 × 10⁷ | <10 | <10 | <10 | <10 |
| 2 | BC | 2.1 × 10⁶ | 2.4 × 10³ | 2.4 × 10³ | 1.4 × 10² | 1.2 × 10³ | 6.6 × 10² |
| 2 | ECOLI | 4.0 × 10⁷ | 2.1 × 10⁶ | 3.3 × 10⁵ | 60 | <10 | <10 |
| 2 | SAZ | 9.3 × 10⁷ | 2.4 × 10⁷ | 1.3 × 10⁷ | 1.7 × 10⁴ | <10 | <10 |
| 2 | KP | 7.1 × 10⁶ | 3.6 × 10⁵ | 1.9 × 10⁵ | <10 | <10 | <10 |
| 2 | ENC | 7.1 × 10⁷ | 1.9 × 10⁸ | 9.3 × 10⁶ | 3.3 × 10⁴ | <10 | <10 |
| 2 | PSA | 2.0 × 10⁶ | 4.7 × 10³ | <10 | <10 | <10 | <10 |
| 3 | CAN | 1.1 × 10⁶ | <10 | <10 | <10 | <10 | <10 |
| 3 | AN | 9.5 × 10² | <10 | <10 | <10 | <10 | <10 |
| 3 | SA | 3.8 × 10⁷ | 6.1 × 10⁴ | <10 | <10 | <10 | <10 |
| 3 | BC | 2.5 × 10⁶ | 1.6 × 10³ | 90 | 250 | <10 | <10 |
| 3 | ECOLI | 3.4 × 10⁷ | 1.6 × 10⁵ | <10 | <10 | <10 | <10 |
| 3 | SAZ | 2.4 × 10⁷ | 7.6 × 10³ | <10 | <10 | <10 | <10 |
| 3 | KP | 2.1 × 10⁶ | <10 | <10 | <10 | <10 | <10 |
| 3 | ENC | 1.6 × 10⁷ | 3.2 × 10³ | <10 | <10 | <10 | <10 |
| 3 | PSA | 2.1 × 10⁵ | <10 | <10 | <10 | <10 | <10 |

Therefore, the data indicated that certain hydrogel-containing medical articles of the invention can be sterilized and imparted antimicrobial properties by loading with a suitable preservative and/or antimicrobial agent such as LIQUID GERMALL® PLUS.

### EXAMPLE 16

### Antimicrobial Activity (Lawn-Based Method)

The antimicrobial properties of the present hydrogel compositions were further tested using a lawn-based method that measured inhibition zones. Blank PEG-soy hydrogels, prepared by the method described in Example 7, were used as controls. Four additional hydrogel compositions were prepared by loading the blank PEG-soy hydrogels with stock solutions (10 mg/ml) of the compounds described in Table 16 below.

TABLE 16
Formulation of hydrogels tested by lawn-based method.

| Formulation | Compound |
|---|---|
| 4 | 3-iodo-2-propynyl N-butylcarbamate (IPBC) |
| 5 | Diazolidinyl urea (50 wt. %) and IPBC (50 wt. %) |
| 6 | Diazolidinyl urea |
| 7 | LIQUID GERMALL ® PLUS |

An aliquot of a frozen bacterial or fungal culture stored at −80° C. in the presence of 9% DMSO was thawed, diluted 5000-fold (approximately 105 CFU) in warm, liquid Mueller-Hinton agar (bacteria; and for S. pyogenes, further supplemented with 5% sheep blood) or Sabouraud dextrose agar (fungi) and poured into Nunc bio-assay dishes (245×245 mm). The thickness of the agar was approximately 4 mm. Small discs (9-10 mm diameter) were cut out of the hydrogels and placed onto the solidified agar. Each composition was tested in triplicate. After incubation at 37° C. for 18 hours, the diameter of the inhibition zones of the hydrogel discs were measured. The results, given in the nearest hundredth of a millimeter, are presented in Table 17 below.

Results

Except for a small inhibition zone of S. pyogenes, the blank gels did not inhibit bacterial growth. Formulation 4 (with IPBC) inhibited growth of S. pyogenes and S. epidermidis CH28, but appeared to be ineffective against the other tested bacteria. There was no difference in size of the inhibition zones of S. pyogenes between the blank gel and Formulation 4, which indicates that IPBC has minimal growth-inhibiting effect on S. pyogenes.

Formulation 5 (containing diazolidinyl urea and IPBC) and Formulation 6 (with diazolidinyl urea alone) inhibited growth of all the bacterial strains tested to approximately the same extent (producing inhibition zones of about 14-23 mm in diameter). Formulation 7 was more effective against most of the tested bacteria compared to both Formulations 5 and 6, although the growth-inhibiting effects of Formulation 7 on S. aureus ATTC 25923, S. pyogenes, E. faecium ATCC 29212, E. coli ATCC 25922, and the various strains of P. aeruginosa and K pneumoniae tested were comparable to those achieved by Formulations 5 and 6.

With regard to yeast and fungi, it was observed that the blank gels were effective enough by themselves to inhibit the growth of C. albicans, C. krusei and especially A. terreus. Formulation 4 also showed fungicidal activity, and the inhibition zones were similar in size compared to those created by Formulation 6. Formulation 5 was observed to be less effective against inhibiting fungal growth than Formulations 4, 6, and 7.

Therefore, the data indicated that certain hydrogel-containing medical articles of the invention can be imparted antimicrobial properties by loading with a suitable preservative and/or antimicrobial agent such as diazolidinyl urea, iodopropynyl butylcarbamate, and/or LIQUID GERMALL® PLUS.

TABLE 17
Antimicrobial properties of hydrogels as tested by lawn-based method.
The diameter of the inhibition zones created by the hydrogel discs
are given in the nearest hundredth of a millimeter.

| | Formulation | | | | |
|---|---|---|---|---|---|
| Microbe | Control | 4 | 5 | 6 | 7 |
| BACTERIA | | | | | |
| S. aureus ATTC 25923 | 0.00 | 0.00 | 22.97 | 22.59 | 23.67 |
| S. aureus 101 | 0.00 | 0.00 | 15.33 | 15.36 | 18.02 |
| S. aureus F170 | 0.00 | 0.00 | 14.20 | 14.81 | 17.99 |
| S. aureus Tokyo 2 | 0.00 | 0.00 | 15.92 | 15.96 | 20.91 |
| S. aureus MRSA 39 | 0.00 | 0.00 | 16.99 | 16.70 | 18.44 |
| S. epidermidis ATCC 12228 | 0.00 | 0.00 | 17.64 | 16.88 | 21.05 |
| S. epidermidis 941 | 0.00 | 0.00 | 14.33 | 16.23 | 20.06 |
| S. epidermidis CH28 | 0.00 | 10.98 | 15.84 | 15.24 | 19.24 |
| S. epidermidis MRSE 70 | 0.00 | 0.00 | 14.70 | 14.21 | 17.69 |
| S. epidermidis H8915 | 0.00 | 0.00 | 15.75 | 14.90 | 20.09 |
| S. pyogenes GAS-1 | 10.84 | 10.82 | 21.05 | 20.88 | 22.88 |
| E. faecalis ATCC 29212 | 0.00 | 0.00 | 14.41 | 14.38 | 15.16 |
| E. faecium VRE-5 | 0.00 | 0.00 | 12.15 | 13.97 | 17.38 |
| P. aeruginosa ATCC 27853 | 0.00 | 0.00 | 15.72 | 15.40 | 14.57 |
| P. aeruginosa PA01 | 0.00 | 0.00 | 15.65 | 15.00 | 15.40 |
| P. aeruginosa D11 | 0.00 | 0.00 | 12.55 | 12.24 | 12.37 |
| P. aeruginosa BF1 | 0.00 | 0.00 | 18.70 | 18.34 | 19.45 |
| K. pneumoniae ATCC 33495 | 0.00 | 0.00 | 18.04 | 17.92 | 19.87 |
| K. pneumoniae OF-3-28-5 | 0.00 | 0.00 | 22.12 | 20.65 | 21.48 |
| K. pneumoniae Tem3 | 0.00 | 0.00 | 17.13 | 16.23 | 17.71 |
| K. pneumoniae CF104 | 0.00 | 0.00 | 18.28 | 17.84 | 18.84 |
| E. coli ATCC 25922 | 0.00 | 0.00 | 16.57 | 16.35 | 17.38 |
| YEAST AND FUNGI | | | | | |
| C. albicans ATCC 90028 | 14.54 | 22.41 | 15.37 | 23.32 | 23.49 |
| C. krusei ATCC 6258 | 12.50 | 19.68 | 17.98 | 21.57 | 23.11 |
| A. terreus 1012 | 17.52 | 33.48 | 26.63 | 35.98 | 39.00 |

### EXAMPLE 17

### Controlled Delivery of Active Agents

Experiments were designed to define the properties of certain hydrogel-containing medical articles of the invention as a drug delivery platform through intact skin. First, the uptake rates of two model active agents, methylene blue and p-nitrophenol were studied. Secondly, the permeation profiles of caffeine as released from a solution versus a hydrogel-containing medical article according to the invention were compared under both occlusive and non-occlusive conditions. In vitro and in vivo hydration studies also were conducted to assess how the swelling of the hydrogels may affect the delivery profile of caffeine. Lastly, different formulations of caffeine-containing and lidocaine-containing medical articles were prepared to assess how the drug delivery properties of these medical articles may be influenced by their drug loading, pH, thickness, protein composition, and the length of the application time.

A. Uptake Rates of Active Agents

To study the uptake rates of active agents, methylene blue and p-nitrophenol, respectively, were loaded into hydrogel samples prepared by a method similar to the method described in Example 7, except that the hydrogel samples used in this study had a thickness of 1 mm.

Blank hydrogel samples were first cut into small squares and allowed to swell and equilibrate in a 10 mM phosphate buffer solution having a pH value of 6 until no p-nitrophenol was detectable by absorbency readings at 400 nm. This was necessary because p-nitrophenol is a by-product that can be produced in both the PEG activation reaction and the polymerization reaction of the activated PEG and the protein, therefore, inaccurate measurements might result if there was a large amount of residual p-nitrophenol present in the hydrogel samples. In their swollen state, the volume of the hydrogels was 745 μl+22 μl.

Uptake solutions of methylene blue (1 ppm) and p-nitrophenol (0.4 wt. %) were prepared. Swollen hydrogel samples were immersed in a beaker containing 90 ml of one of the uptake solutions for 1.50 minutes, 3 minutes, 6 minutes, 15 minutes, 30 minutes, and 60 minutes before they were removed from the solution. The hydrogels were then carefully blotted of excess solution and were each transferred into a second beaker containing 30 ml of a 10 mM phosphate buffer solution with a pH of 6 to equilibrate.

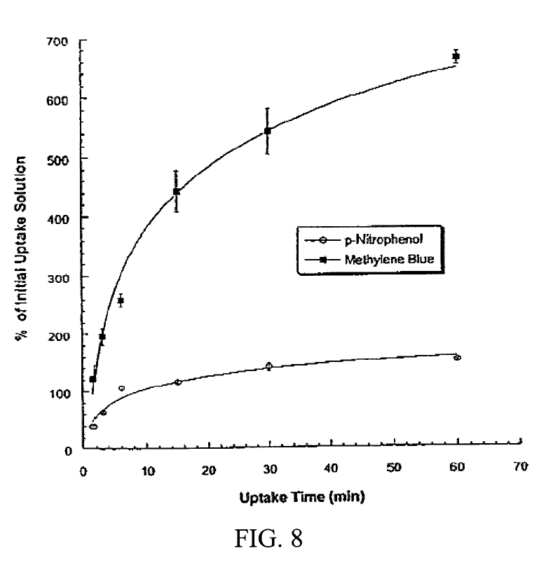

The hydrogels were allowed to equilibrate in the buffer solution for 24 hours. The hydrogels were continuously agitated to ensure that the equilibrium state was reached. The uptake of p-nitrophenol and methylene blue was assumed to correspond to the amount that was released into the washing buffer solution. The amount of p-nitrophenol in the washing buffer solution was measured by absorbency readings taken at 400 nm and comparing the results to a standard curve in the range of 1 μg/ml to 80 μg/ml. Methylene blue was similarly measured at 655 nm and the calibration curve was in the range of 0.0025 ppm to 3 ppm. To evaluate the relative uptake of either of these model molecules, the total quantity of molecules in the 30-ml solution was taken to correspond to the initial volume of the hydrogel (745 μl). The concentration of the model molecules reported in the hydrogel was then compared (in percentage) to the initial concentration of the uptake solution. FIG. 8 shows the percentage of the initial uptake solution of p-nitrophenol and methylene blue as a function of time.

Results

As shown in FIG. 8, both molecules diffused very rapidly into the hydrogel samples and reached the same concentration as the uptake solution in less than 1.50 minutes for methylene blue and in about 15 minute for p-nitrophenol. In the case of methylene blue, it was observed that the hydrogel could be loaded to a concentration multiple times greater than the concentration of the initial uptake solution within a relatively short time. For example, it was observed that the hydrogel became 6 times more concentrated than the initial uptake solution within an hour. This phenomenon may be caused by the latent charge of the hydrogel or the natural affinity of methylene blue for protein. As many other active agents have affinities to protein, it can be expected that the hydrogels of the invention can be loaded with a high concentration of a variety of active agents within a relatively short time.

B. Hydrogel-Containing Medical Articles as a Topical Delivery System of Active Ingredients

The experiments described in this section were designed to define the properties of the hydrogel-containing medical articles of the invention as a drug delivery platform through intact skin. Caffeine was used as model permeant to assess the hydrogel-induced penetration profile. Caffeine is a relatively polar compound with low solubility either in water (22 mg/ml) or in oil, commonly used in cosmetic products. Such a property is characteristic of many other natural compounds that can be used as valuable cosmetic active ingredients.

1. In Vitro Permeation Study

To compare the delivery profiles of caffeine as released from a solution versus from a hydrogel-containing medical article according to the invention, hydrogels prepared by the method described in Example 7 were soaked in a 2% (by weight) caffeine (SigmaUltra grade from Sigma-Aldrich Chemical Co., Milwaukee, Wis.) solution for 1 hour at room temperature under gentle agitation. The caffeine solution further contained EDTA (0.2 wt. %) and NaH₂PO₄ (0.16 wt. %). A second impregnation was performed in the same solution overnight. The loaded hydrogels were then cut into circular pieces having a diameter of 9 mm, and kept in solution until their application onto porcine skin. The integration volume represented 10 times the volume of the dehydrated hydrogels. The hydrogels had a pH of 5.5.

After cleaning with cold tap water, porcine skin was shaved and then stored frozen in aluminum foil at −20° C. Before use, the skin was thawed and then dermatomed to a thickness of 510 μm with a Padgett Electro-Dermatome (Padgett Instrument Inc, Kansas City, Mo.). Percutaneous absorption was measured using 0.9 cm-diameter horizontal glass diffusion cells consisting of a donor (where the tested sample is applied) and a receptor (where a tested active might diffuse to) compartments (OECD guidelines, 2000). Such cells, known as Franz-type diffusion cells, or static cells, were supplied by Logan Instrument Corp (Somerset, N.J.). Dermatomed porcine skin samples were cut with surgical scissors and placed between the two halves of a diffusion cell, with stratum corneum facing the donor chamber. The area available for diffusion was 0.635 cm², and the receptor phase was 4.5 ml.

The receptor chamber was filled with 0.22 μm-filtered phosphate saline buffer (pH 7.4) containing 20% (v/v) ethanol and allowed to equilibrate to the needed temperature. Temperature of the skin surface was maintained at 37° C. throughout the experiment by placing diffusion cells into a dry block heater set to 37° C. The receptor compartment contents were continuously agitated by small PTFE-coated magnetic stirring bars.

Skin samples were allowed to equilibrate with receptor medium for at least one hour before application of test formulations. Groups were randomized, and hydrogels that had been loaded with 2% (by weight) caffeine solutions (described above) were applied to a first set of test cells. A second set of test cells were filled with 2% (by weight) caffeine solutions. The experiment was performed under both non-occlusive and occlusive conditions to assess the effect of occlusion.

Receptor fluid was removed at predetermined times (2 hours, 4 hours, 6 hours, and 8 hours) and replaced with fresh temperature-equilibrated buffer. The removed receptor fluids were assayed to determine the amount of caffeine that was delivered to the receptor cell at given times. At the end of the experiment (i.e., at 24 hours), receptor fluid was again removed and assayed. Additionally, hydrogels were removed from the skin surface and placed in a methanol/water mixture (20/80; v/v) overnight at room temperature to allow caffeine extraction. The donor cells were then washed exhaustively with ethanol. The exposed skin was excised, and the epidermis was separated from the dermis. The skin strata were placed in a methanol/water mixture (80/20; v/v) for 48 hours at room temperature. All samples (receptor fluid, epidermis, dermis, hydrogel, washings) were assayed by high performance liquid chromatography (HPLC) for mass balance verification.

The parameters for the HPLC setup were as follows. The HPLC instrumentation consisted of an Agilent 1050 quaternary LC module equipped with a variable wavelength detector set at 272 nm, a column, an oven, an in-line degasser, and an automated sample injector. The column, an L1 USP type (ACE 5 C18, pore size 100 Å, 15 cm×4 mm i.d.) was used at room temperature. The flow rate was maintained constant at 1.5 ml/min. The injected volume was 10 μl, and the mobile phase was 20% methanol and 80% 0.05 M phosphate buffer in deionized water (pH 3.5 with phosphoric acid). The run time was 7 minutes. Under these conditions, the caffeine retention time ranged between 3.2 and 3.4 minutes.

The caffeine concentration in each sample was determined, individually, against a 6-point linear calibration curve. Standard caffeine solutions with concentrations of 50 μg/ml, 100 μg/ml, 200 μg/ml, 300 μg/ml, 500 μg/ml, and 1000 μg/ml were prepared by successive dilutions of a 1 mg/ml caffeine stock solution with mobile phase. Each standard caffeine solution was injected in triplicate.

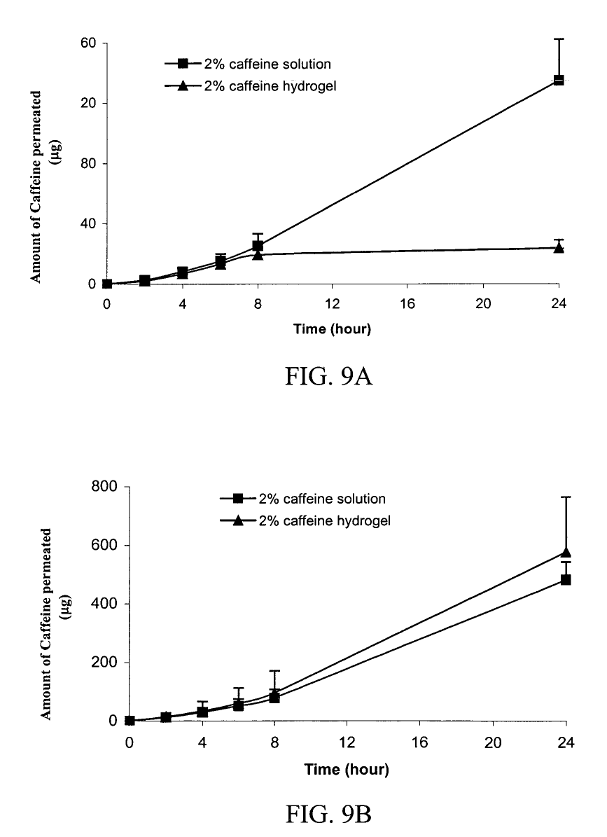

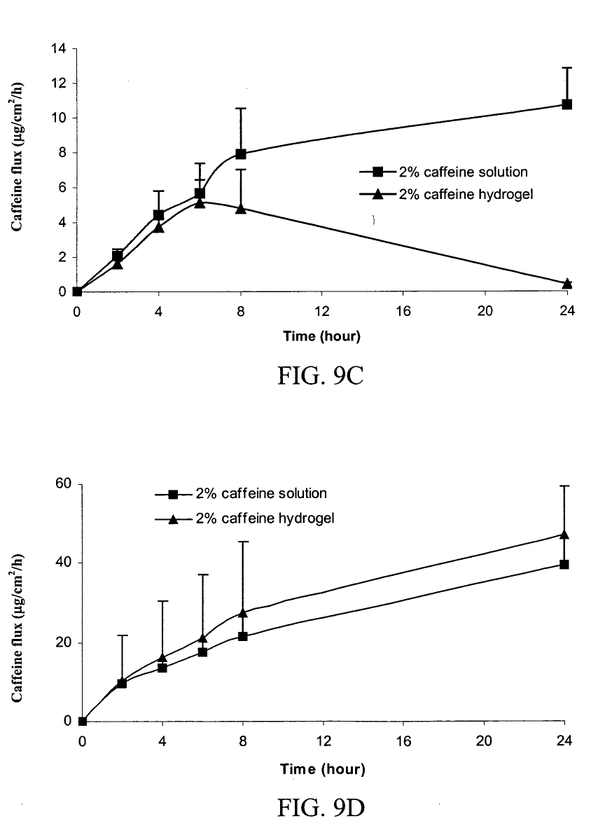

The chromatograms obtained were used to calculate the total cumulative amount of caffeine recovered in each compartment (hydrogel, washing, epidermis, dermis, and receptor fluid). Results were presented in Table 18 and FIGS. 9A to 9D. Table 18 summarizes the cumulative amounts of caffeine that were recovered in the different compartments at the end of the 24-hour period under the different experimental conditions. For each experimental condition, the experiment was conducted at least 5 times to obtain the average value presented in Table 18. FIGS. 9A-D represent the corresponding caffeine permeation profiles versus time. FIGS. 9A and 9B show the cumulative amounts of caffeine permeated across the porcine skin samples (i.e., recovered from the receptor fluid) over 24 hours, measured in micrograms, under non-occlusive (FIG. 9A) and occlusive conditions (FIG. 9B), respectively. FIGS. 9C and 9D show the flux of caffeine (calculated as the amount of caffeine permeated across the area of porcine skin per hour in 1 g/cm²/h) as a function of time under non-occlusive (FIG. 9C) and occlusive conditions (FIG. 9D), respectively.

Results

As shown in Table 18, under both occlusive and non-occlusive conditions, and regardless of the formulation applied, most of the caffeine applied remained either on the skin surface (as indicated by the amount recovered from the washings) or within the hydrogel. Moreover, it was observed that very little caffeine was absorbed in either the epidermis or the dermis.

As shown in FIGS. 9A and 9C, it was observed that, for the first six hours of the study, the amount of caffeine permeated across the porcine skin samples was similar under non-occlusive conditions regardless of whether the caffeine was delivered from the solution or via the hydrogel. However, beyond the sixth hour, caffeine delivery via the hydrogel began to slow down and eventually stopped before the end of the 24-hour period. This may be seen from the continually decreasing flux after the sixth hour as shown in FIG. 9C. By comparison, as shown in FIGS. 9A and 9C, caffeine, when released from a solution, continued to permeate across the porcine skin until the end of the test period, and the flux also continued to increase (albeit at a slower rate after the either hour) until the end of the 24-hour period.

Without being bound by any theory, it is believed that the decrease of caffeine flux over time observed with the hydrogel was due to water depletion. As the hydrogel becomes dehydrated under non-occlusive conditions, its ability to deliver active agents, such as caffeine, may decrease. This is supported by the results obtained from the experiments conducted under occlusive conditions. As shown in FIGS. 9B and 9D, the amount of caffeine delivered as well as the flux across the porcine skin were very similar under occlusive conditions regardless of whether the caffeine was delivered from the solution or via the hydrogel throughout the entire 24-hour period. These results suggest that the hydrogels according to the invention, as long as they are hydrated (e.g., by occlusion), do not represent a limiting factor for caffeine delivery. In fact, the hydrogels that were studied under occlusion behaved like an infinite reservoir of caffeine and were able to afford sustained delivery of caffeine over the 24-hour period.

From the data obtained in this experiment, it can be concluded that hydrogel-containing medical articles of the invention are capable of sustained delivery of active agents (e.g., caffeine), provided that the hydrogel stays hydrated. Occlusive conditions of application may prevent dehydration of the hydrogel, thus providing longer times of drug delivery.

TABLE 18
Caffeine delivery by solution versus via hydrogel. Each value represents the average cumulative amount
of caffeine in μg (and % applied dose) recovered in the different compartments at the end of the
24-hour test period. The average value presented was obtained from at least five samples.

| | | RECEPTOR FLUID | EPIDERMIS | DERMIS | WASHING | HYDROGEL | MASS BALANCE |
|---|---|---|---|---|---|---|---|
| Non-occlusive | | | | | | | |
| Solution | μg | 134.30 ± 27.44 | 3.88 ± 0.71 | 5.43 ± 3.95 | 2013.38 ± 143.11 | — | 2157.00 ± 143.11 |
| | (%) | (5.86 ± 1.20) | (0.17 ± 0.03) | (0.24 ± 0.17) | (87.80 ± 6.24) | | (94.06 ± 6.28) |
| Hydrogel | μg | 23.53 ± 5.50 | 5.98 ± 6.26 | 4.67 ± 4.82 | 493.34 ± 1230.15 | 1769.58 ± 177.43 | 2296.00 ± 369.00 |
| | (%) | (0.95 ± 0.22) | (0.24 ± 0.25) | (0.19 ± 0.19) | (19.85 ± 230.15) | (71.21 ± 7.14) | (92.40 ± 14.86) |
| Occlusive | | | | | | | |
| Solution | μg | 481.06 ± 60.50 | 5.72 ± 0.92 | 20.27 ± 5.01 | 1986.62 ± 281.84 | — | 2494.00 ± 283.00 |
| | (%) | (18.01 ± 2.27) | (0.21 ± 0.04) | (0.76 ± 0.19) | (74.39 ± 10.55) | | (93.38 ± 10.59) |
| Hydrogel | μg | 575.67 ± 188.45 | 15.64 ± 4.83 | 29.26 ± 7.85 | 507.00 ± 174.18 | 2054.51 ± 309.28 | 3182.00 ± 261.00 |
| | (%) | (17.76 ± 5.81) | (0.48 ± 0.15) | (0.90 ± 0.24) | (15.64 ± 5.37) | (63.37 ± 9.54) | (98.15 ± 8.04) |

2. Water Content of Hydrogel Samples

Pre-weighed hydrogel samples, prepared as described in Example 7, were loaded with 2%, 1%, 0.5% and 0% (by weight) caffeine (SigmaUltra grade from Sigma-Aldrich Chemical Co., Milwaukee, Wis.) solution using the methodology described in Part 1 above. The loaded hydrogel samples were then applied onto porcine skin in vitro under non-occlusive and occlusive conditions. The temperature of the porcine skin was maintained at 32° C.

Hydrogel samples were collected and weighed (Wₛ) after 2, 4, 6, 8, and 24 hours at 32° C. The weight of dry hydrogel samples (W₀) was determined after dehydration of the hydrogel at 60° C. for 4 hours. Each weight measurement was taken three times and the average was used to calculate the water content (C_{w}) of the hydrogels in accordance with equation (1) above.

Results

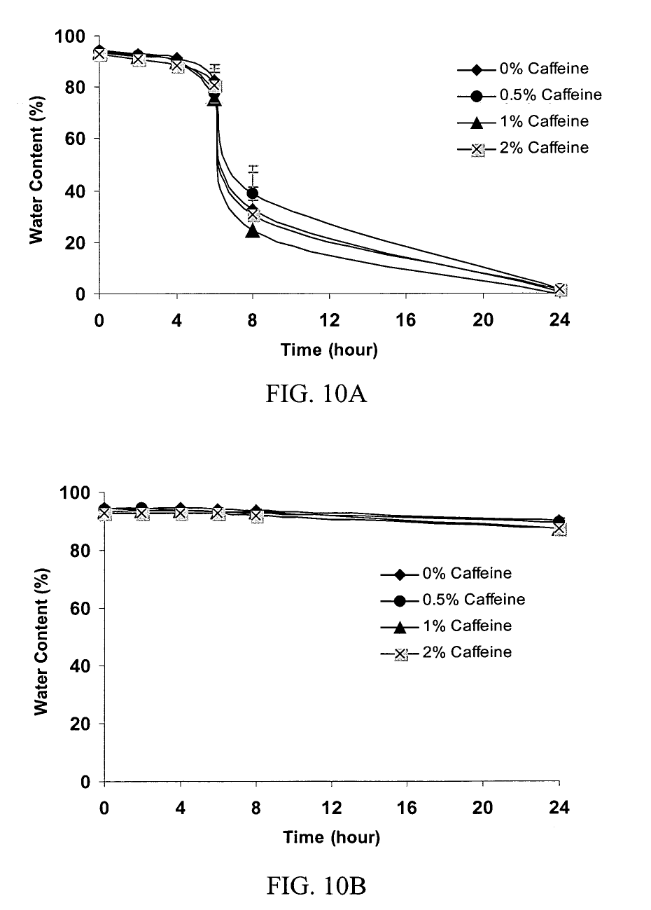

FIGS. 10A and 10B show the water content of the hydrogel samples as applied on the skin under non-occlusive (FIG. 10A) and occlusive (FIG. 10B) conditions. Under non-occlusive conditions, the water content of the hydrogel samples decreased significantly after the first 6 hours and became completely dried up at the end of the 24-hour period. Under occlusive conditions, the water content of the hydrogel samples did not decrease significantly over a 24 hour period. In fact, each of the four tested hydrogel samples retained a water content of about at least 90% at the end of the test period. Additionally, it was observed that drug loading did not affect the water content of hydrogels, under both non-occlusive and occlusive conditions.

3. In Vivo Hydration Study

To evaluate the in vivo hydrating effect of hydrogels according to the invention, hydrogels prepared as described in Example 7 were loaded with 0%, 0.5%, 1%, and 2% (by weight) caffeine solution using the methodology described in Part 1 above. Twelve male and female human subjects were enrolled in the study after verification of inclusion and exclusion criteria. After 15 minutes of acclimatization (T₀) at 20° C.±2° C. and 45%±5% relative humidity, the hydration level of the dermal site where the hydrogel was to be applied was measured as described below. Test products were randomly applied on the upper volar part of either arm under non-occlusive and occlusive conditions and kept in place for 2 hours (for the non-occlusive study) and 24 hours (for the occlusive study), respectively.

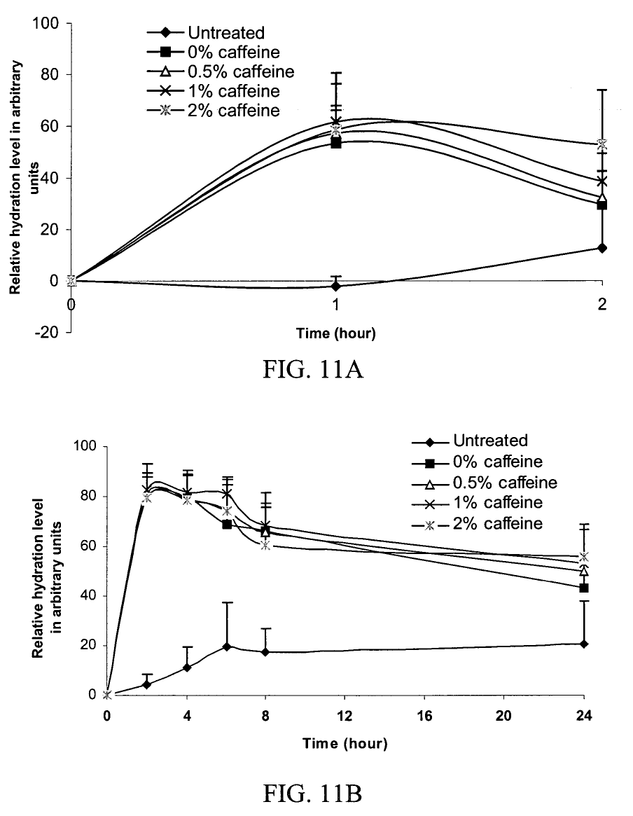

Skin hydration levels were measured with a Corneometer® CM825 device (Courage and Khazaka, Germany) equipped with a 49 mm² probe as described in Example 14. To account for the variation of hydration level at different sites of the skin, application of the different samples was randomized, and three consecutive measurements were taken on each skin area for each volunteer. For each skin area, relative hydration level was calculated at time Tₙ in accordance with equation (3) below:
Relative hydration level=Capacitance at Tₙ−Capacitance at T₀ (3)
For the non-occlusive study, hydration measurements were taken at the first and second hours (Tₙ=T₁ₕ and T₂ₕ). For the occlusive study, hydration measurements were taken at the second, fourth, and twenty-fourth hour (Tₙ=T₁ₕ, T₂ₕ, and T₂₄ₕ). Absolute skin hydration levels as measured in capacitance (expressed in arbitrary units) after the first 2 hours of application of the caffeine-containing hydrogel samples are summarized in Table 19 below. FIGS. 11A and B show the relative skin hydration levels as determined by equation (3) above under non-occlusive (FIG. 11A) and occlusive conditions (FIG. 11B), respectively.
Results

As shown in Table 19, it was observed that, regardless of the drug loading, the tested hydrogel samples were able to induce a significant increase in skin hydration level after a 2-hour application under both non-occlusive and occlusive conditions. Under occlusive conditions, skin hydration appeared to be maximized.

TABLE 19
Absolute skin hydration levels as measured in capacitance
(expressed in arbitrary units) after a 2 hour-application
of caffeine-containing hydrogels under non-occlusive and
occlusive conditions. (Means ± Sd, n = 12).

| | NON-OCCLUSIVE | OCCLUSIVE |
|---|---|---|
| Caffeine-containing | | |
| hydrogels | | |
| 0% caffeine | 61.89 ± 13.99 | 109.28 ± 5.80 |
| 0.5% caffeine | 61.67 ± 13.34 | 109.44 ± 3.63 |
| 1% caffeine | 67.89 ± 11.05 | 109.89 ± 3.71 |
| 2% caffeine | 85.97 ± 12.58 | 107.72 ± 5.22 |
| Untreated area | 32.97 ± 14.83 | 32.69 ± 6.16 |

As shown in FIG. 11A, regardless of the drug loading, there was an increase in skin hydration level over the 2-hour test period under non-occlusive conditions, although the increase became smaller after the first hour of application possibly due to the loss of water in the hydrogel samples and/or the loss of adherence of the hydrogel samples to the skin. As shown in FIG. 11B, a significant increase in skin hydration level was observed for each of the four hydrogel formulations under occlusive conditions. The increase was sustained over the first 8 hours of the test period, after which the increase in skin hydration level became less significant.

4. Conclusion

From the data obtained from the different experiments described in this example, it can be concluded that medical articles containing the tested hydrogels are good candidates for delivering hydrophilic drug through the skin. The experiments further showed that caffeine was readily available for release when the hydrogels were loaded with a 2% (by weight) caffeine solution, and its permeation across porcine skin was measurable as early as 2 hours after the application of the hydrogels. Additionally, it was observed that the permeation of caffeine through the skin was effected by the swelling of the hydrogels. Therefore, the results from these studies demonstrate that the presence of water within the hydrogel is beneficial to achieve an effective cutaneous drug release, which is further accompanied by optimal hydration of the skin.

C. Influence of Various Parameters on Drug Delivery Via Hydrogel-Containing Medical Articles

1. Caffeine Delivery Via Hydrogel-Containing Medical Articles

a. Influence of Drug Loading

To assess the influence of drug loading on caffeine delivery via hydrogel-containing medical articles of the invention, hydrogel samples were prepared according to the method described and Example 7 and loaded with 0.5%, 1%, and 2% (by weight) caffeine (SigmaUltra grade from Sigma-Aldrich Chemical Co., Milwaukee, Wis.) solution. The loaded hydrogels were then applied to Franz-type diffusion cells containing porcine skin samples as described in Section B, Part 1, above. Receptor fluid was totally removed and replaced at 2 hours, 4 hours, 6 hours, and 8 hours. The removed receptor fluid was assayed to determine the amount of caffeine that had been delivered to the receptor cell. Caffeine was extracted from the various compartments of the cells (receptor fluid, hydrogel, epidermis, dermis, washings) at the end of the 24-hour test period. This experiment was conducted under both occlusive and non-occlusive conditions.

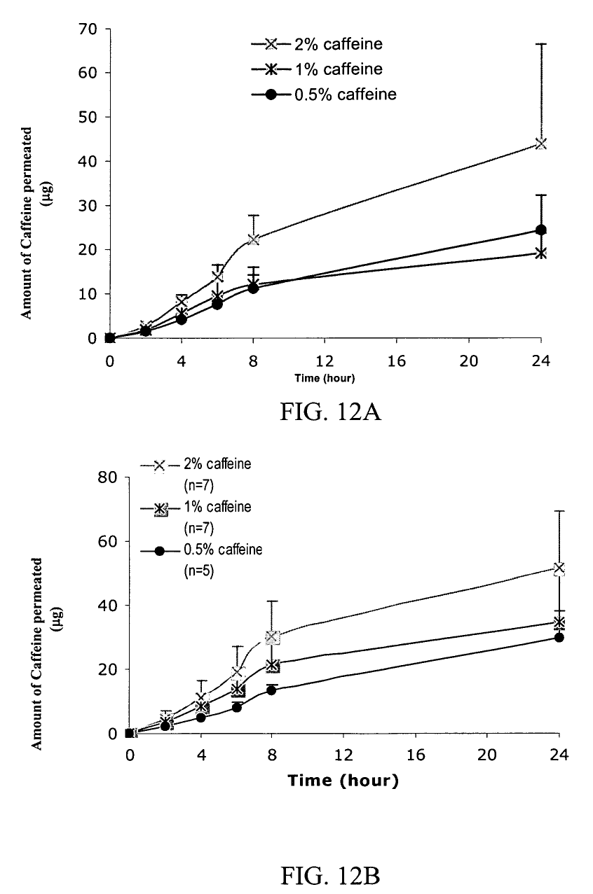

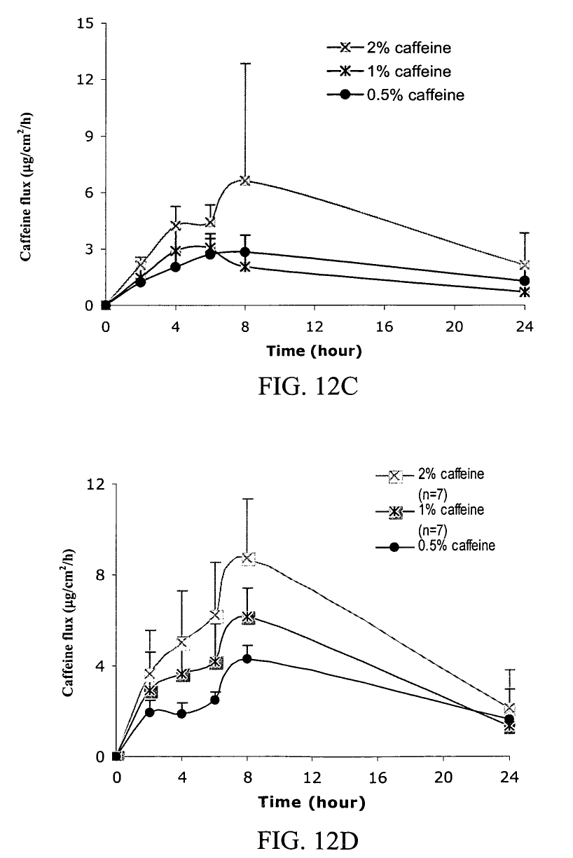

Table 20 summarizes the cumulative amounts of caffeine that were recovered in the different compartments at the end of the 24-hour test period under the different experimental conditions. For each experimental condition, the experiment was conducted on at least five samples to obtain the average value presented in Table 20. FIGS. 12A-D represent the corresponding caffeine permeation profiles as a function of time. FIGS. 12A and 12B show the cumulative amount of caffeine permeated across the porcine skin samples (i.e., recovered from the receptor fluid) over the 24-hour test period under non-occlusive (FIG. 12A) and occlusive conditions (FIG. 12B), respectively. FIGS. 12C and 12D show the flux of caffeine (calculated as the amount of caffeine permeated across the area of porcine skin per hour in μg/cm²/h) as a function of time under non-occlusive (FIG. 12C) and occlusive conditions (FIG. 12D), respectively.

Results

As shown in Table 20, under both occlusive and non-occlusive conditions, and regardless of the formulation applied, most of the caffeine applied remained either on the skin surface (as indicated by the amount recovered from the washings) or within the hydrogel. Moreover, it was observed that very little caffeine was absorbed in either the epidermis or the dermis.

As shown in FIG. 12A, under non-occlusive conditions, the medical article including a hydrogel that had been loaded with a 2% (by weight) caffeine solution delivered significantly larger amount of caffeine than its 1% and 0.5% counterparts. Between the 1% and 0.5% formulations, there was no significant difference in the amount of caffeine that each of them delivered.

As shown in FIG. 12B, under occlusive conditions, the 2% formulation delivered significantly larger amount of caffeine than the 0.5% formulation. No significant difference could be found between the 1% and 2% or 0.5% formulations.

TABLE 20
Influence of drug loading on caffeine permeation profiles as released from hvdrogel-containing medical
articles under non-occlusive and occlusive conditions. Each value represents the average cumulative amount
of caffeine in μg (and % applied dose) recovered in the different compartments at the end of the 24-hour
test period. The 5 average value presented was obtained from at least five samples.

| | | RECEPTOR | | | | | MASS |
|---|---|---|---|---|---|---|---|
| | | FLUID | EPIDERMIS | DERMIS | WASHING | HYDROGEL | BALANCE |
| NON-OCCLUSIVE | | | | | | | |
| *2% Caffeine | μg | 43.77 ± 22.55 | 13.76 ± 13.52 | 7.37 ± 3.94 | 349.41 ± 348.55 | 1614.25 ± 549.17 | 2028.56 ± 0.20 |
| Hydrogel | % | 2.13 ± 1.09 | 0.67 ± 0.66 | 0.36 ± 0.19 | 16.97 ± 16.92 | 78.38 ± 26.66 | 98.50 ± 9.91 |
| *1% Caffeine | μg | 19.05 ± 4.56 | 5.64 ± 1.22 | 3.55 ± 0.81 | 87.98 ± 25.12 | 1083.20 ± 102.31 | 1199.42 ± 0.08 |
| Hydrogel | % | 1.56 ± 0.37 | 0.46 ± 0.10 | 0.29 ± 0.07 | 7.22 ± 2.06 | 88.87 ± 8.39 | 98.40 ± 6.48 |
| †0.5% Caffeine | μg | 24.27 ± 7.92 | 4.83 ± 0.78 | 3.89 ± 0.97 | 123.67 ± 114.27 | 434.10 ± 239.53 | 590.77 ± 0.13 |
| Hydrogel | % | 4.20 ± 1.37 | 0.84 ± 0.14 | 0.67 ± 0.17 | 21.41 ± 19.79 | 75.17 ± 41.48 | 102.30 ± 22.58 |
| OCCLUSIVE | | | | | | | |
| *2% Caffeine | μg | 51.35 ± 18.12 | 9.94 ± 3.84 | 14.32 ± 4.36 | 524.19 ± 102.04 | 1812.03 ± 179.99 | 2411.84 ± 162.25 |
| Hydrogel | % | 1.83 ± 0.64 | 0.35 ± 0.14 | 0.51 ± 0.16 | 18.64 ± 3.63 | 64.43 ± 6.40 | 85.76 ± 5.77 |
| *1% Caffeine | μg | 34.88 ± 15.84 | 7.09 ± 2.29 | 9.89 ± 2.36 | 251.59 ± 94.66 | 954.99 ± 121.15 | 1258.44 ± 64.18 |
| Hydrogel | % | 2.49 ± 1.13 | 0.51 ± 0.16 | 0.70 ± 0.17 | 17.93 ± 6.75 | 68.05 ± 8.63 | 89.68 ± 4.57 |
| °0.5% Caffeine | μg | 29.72 ± 8.55 | 5.52 ± 0.93 | 6.04 ± 1.40 | 123.44 ± 39.74 | 486.79 ± 55.38 | 651.51 ± 14.48 |
| Hydrogel | % | 3.79 ± 1.09 | 0.70 ± 0.12 | 0.77 ± 0.18 | 15.75 ± 5.07 | 62.11 ± 7.07 | 83.12 ± 1.85 |

*n = 7
†n = 6
°n = 5

Data in FIGS. 12C and 12D indicated that, under both non-occlusive and occlusive conditions, and regardless of the formulation tested, the caffeine flux slowly increased and reached a maximum between the sixth and eighth hours, which was followed by a marked decrease at the end of the 24-hour test period. Without being bound by any theory, it is believed that water evaporated from the different formulations, thereby slowing down the delivery rate of caffeine. Although these observations are consistent with the conclusion made in Part B above regarding non-occlusive systems, they are not consistent for the occlusive study. In fact, it was observed that the hydrogels tested in the occlusive study were 30%-60% dry at the end of the 24-hour test period. It is believed that an ineffective occlusive system had led to these observations.

Nevertheless, from the data obtained in this experiment, it can be concluded that among the three concentrations studied, the 2% formulation offered the most efficient delivery.

b. Influence of pH

To assess the influence of pH on caffeine delivery via hydrogel-containing medical articles of the invention, hydrogel samples prepared according to the method described in Example 7 were buffered to adjust their pH to 3.0, 5.5, and 9.0. The hydrogel samples were subsequently loaded with 0.5% and 2% (by weight) caffeine (SigmaUltra grade from Sigma-Aldrich Chemical Co., Milwaukee, Wis.) solution, then applied to a Franz-type diffusion cell containing a porcine skin sample as described in Part B above. Receptor medium was totally removed and replaced at 2 hours, 4 hours, 6 hours, and 8 hours. The removed receptor medium was assayed to determine the amount of caffeine that was delivered to the receptor cell at a given time. Caffeine was extracted from the various other compartments of the cells at 24 hours. This experiment was conducted under both occlusive and non-occlusive conditions.

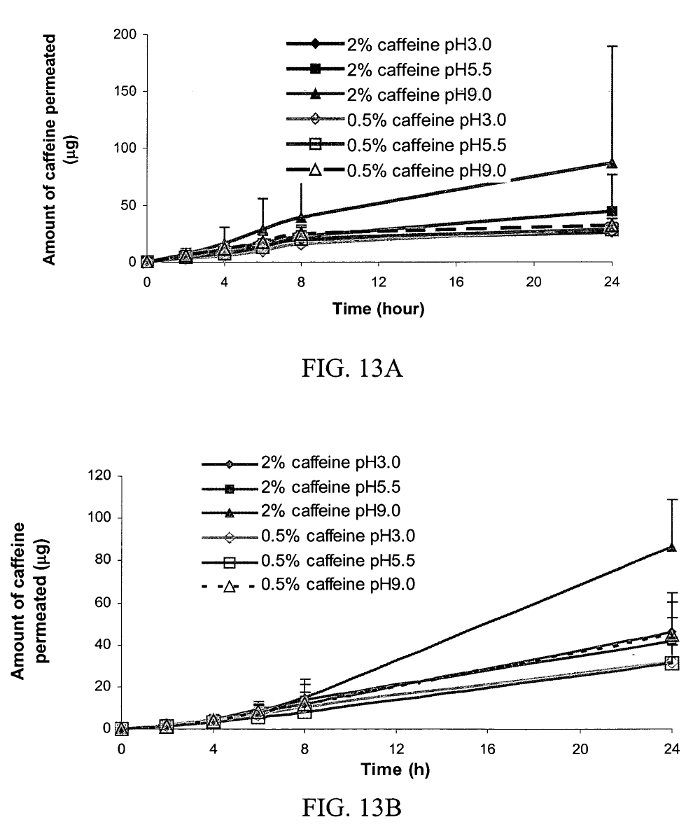

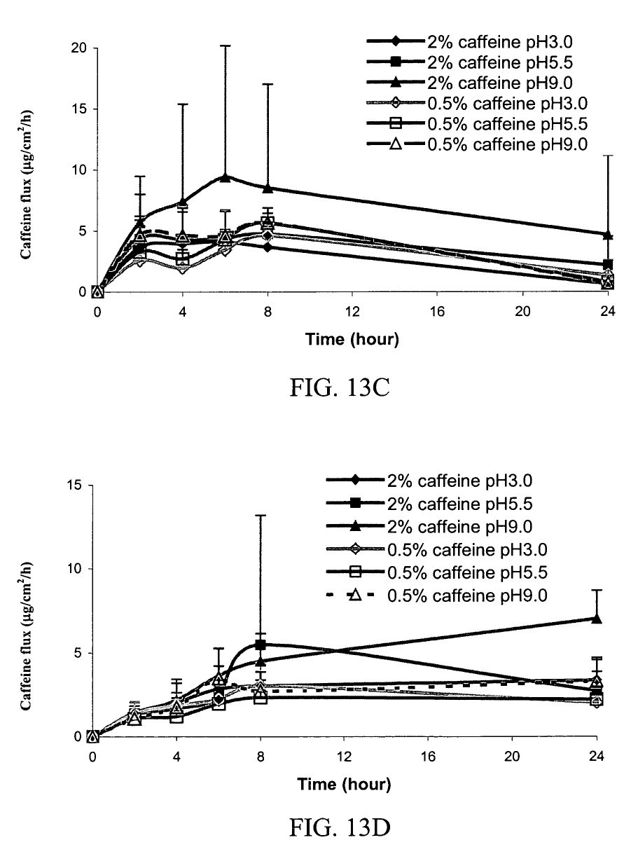

Table 21 summarizes the cumulative amounts of caffeine that were recovered in the different compartments at the end of the 24-hour test period under the different experimental conditions. For each experimental condition, the experiment was conducted on at least 6 samples to obtain the average value presented in Table 21. FIGS. 13A to 13D represent the corresponding caffeine permeation profiles versus time. FIGS. 13A and 13B show the cumulative amounts of caffeine permeated across the porcine skin samples (i.e., recovered from the receptor medium) over 24 hours under non-occlusive (FIG. 13A) and occlusive conditions (FIG. 13B), respectively. FIGS. 13C and 13D show the flux of caffeine (calculated as the amount of caffeine permeated across the area of porcine skin per hour in μg/cm²/h) as a function of time under non-occlusive (FIG. 13C) and occlusive conditions (FIG. 13D), respectively.

Results

As shown in Table 21, under both occlusive and non-occlusive conditions, and regardless of the formulation applied, most of the caffeine applied remained either on the skin surface (as indicated in the amount recovered from the washings) or within the hydrogel. Moreover, it was observed that only a very small amount of caffeine was absorbed in the epidermis or the dermis.

It was observed that under non-occlusive conditions, changes in pH did not seem to have a significant effect on the amount of caffeine that permeated across the skin under the experimental conditions used. Specifically, no statistical difference (p>0.05) was observed at 24 hours between the amount of caffeine that permeated across the porcine skin samples regardless of the caffeine concentration or the pH of the hydrogels. The data indicated a weak positive correlation between the amount of caffeine that was permeated and the pH value of the hydrogels, but the correlation was not significant.

It was observed that under occlusive conditions, the medical articles with a hydrogel having a pH value of 9.0 were able to deliver a larger amount of caffeine than the lower pH formulations. Additionally, the formulation with a pH of 9.0 that had been loaded with a 2% (by weight) caffeine solution was found to be more efficient in delivering caffeine than the formulation with a pH of 9.0 that had been loaded with a 0.5% (by weight) caffeine solution. It was further observed that no statistical difference could be found between the pH 3.0 and pH 5.5 formulations regardless of the caffeine concentration used.

TABLE 21
Influence of pH on caffeine permeation profiles as released from hydrogel-containing medical articles according to the invention.
Each value represents the average cumulative amount of caffeine in μg (and % applied dose) recovered in the different compartments
at the end of the 24-hour test period. The average value presented was obtained from at least six samples.

| | | 2% caffeine | | 2% caffeine | | 2.0% caffeine | | 0.5% caffeine | | 0.5% caffeine | | 0.5% caffeine | |
|---|---|---|---|---|---|---|---|---|---|---|---|---|---|
| | | (pH 3.0) | | (pH 5.5) | | (pH 9.0) | | (pH 3.0) | | (pH 5.5) | | (pH 9.0) | |
| | | n = 8 | | n = 8 | | n = 6 | | n = 8 | | n = 8 | | n = 6 | |
| NON-OCCLUSIVE | | Avg | ±Sd | Avg | ±Sd | Avg | ±Sd | Avg | ±Sd | Avg | ±Sd | Avg | ±Sd |
| Receptor | μg | 25.84 | 12.43 | 44.61 | 32.06 | 86.76 | 102.27 | 29.46 | 14.91 | 28.39 | 4.18 | 32.15 | 2.49 |
| Fluid | % | 1.49 | 0.72 | 2.29 | 1.65 | 4.59 | 5.40 | 5.35 | 2.71 | 5.28 | 0.78 | 5.67 | 0.44 |
| Epidermis | μg | 4.62 | 2.90 | 5.48 | 4.14 | 5.70 | 4.55 | 1.58 | 0.95 | 0.93 | 0.53 | 0.72 | 0.21 |
| | % | 0.27 | 0.17 | 0.28 | 0.21 | 0.30 | 0.24 | 0.29 | 0.17 | 0.17 | 0.10 | 0.13 | 0.04 |
| Dennis | μg | 4.30 | 2.58 | 5.40 | 4.64 | 8.77 | 9.98 | 2.40 | 2.11 | 1.12 | 0.92 | 1.22 | 0.64 |
| | % | 0.25 | 0.15 | 0.28 | 0.24 | 0.46 | 0.53 | 0.44 | 0.38 | 0.21 | 0.17 | 0.22 | 0.11 |
| Hydrogel | μg | 1853.4 | 795.0 | 1359.2 | 347.1 | 1316.2 | 398.5 | 405.5 | 101.6 | 451.3 | 25.8 | 469.5 | 13.5 |
| | % | 106.68 | 45.76 | 69.89 | 17.85 | 69.56 | 21.06 | 73.62 | 18.45 | 84.00 | 4.80 | 82.77 | 2.38 |
| Washings | μg | 115.1 | 53.7 | 225.6 | 198.5 | 257.5 | 235.2 | 50.4 | 58.7 | 24.7 | 9.5 | 29.5 | 11.6 |
| | % | 6.63 | 3.09 | 11.60 | 10.21 | 13.61 | 12.43 | 9.15 | 10.65 | 4.59 | 1.77 | 5.19 | 2.04 |
| Mass | μg | 2003.2 | 784.5 | 1640.3 | 158.9 | 1674.9 | 88.9 | 489.3 | 34.9 | 506.4 | 23.1 | 533.1 | 219.0 |
| balance | % | 115.30 | 45.15 | 84.35 | 8.17 | 88.52 | 4.70 | 88.84 | 6.33 | 94.26 | 4.30 | 93.97 | 3.86 |

| | | | | | |
|---|---|---|---|---|---|
| 2% caffeine | 2% caffeine | 2.0% caffeine | 0.5% caffeine | 0.5% caffeine | 0.5% caffeine |
| (pH 3.0) | (pH 5.5) | (pH 9.0) | (pH 3.0) | (pH 5.5) | (pH 9.0) |
| n = 8 | n = 8 | n = 7 | n = 8 | n = 8 | n = 8 |

| OCCLUSIVE | | Avg | ±Sd | Avg | ±Sd | Avg | ±Sd | Avg | ±Sd | Avg | ±Sd | Avg | ±Sd |
|---|---|---|---|---|---|---|---|---|---|---|---|---|---|
| Receptor | μg | 46.07 | 18.33 | 41.47 | 11.28 | 86.20 | 22.84 | 31.41 | 12.05 | 30.88 | 8.74 | 45.05 | 15.37 |
| Fluid | % | 2.30 | 0.92 | 1.88 | 0.51 | 4.19 | 1.11 | 5.37 | 2.06 | 5.26 | 1.49 | 7.21 | 2.46 |
| Epidermis | μg | 61.09 | 75.73 | 31.69 | 42.41 | 22.36 | 12.59 | 4.41 | 2.31 | 6.14 | 2.01 | 7.41 | 2.76 |
| | % | 3.05 | 3.78 | 1.44 | 1.92 | 1.09 | 0.61 | 0.75 | 0.39 | 1.05 | 0.34 | 1.19 | 0.44 |
| Dermis | μg | 13.32 | 19.69 | 14.33 | 2.66 | 21.41 | 11.77 | 3.95 | 1.42 | 5.16 | 1.92 | 6.90 | 3.48 |
| | % | 0.66 | 0.98 | 0.65 | 0.12 | 1.04 | 0.57 | 0.68 | 0.24 | 0.88 | 0.33 | 1.10 | 0.56 |
| Hydrogel | μg | 1374.5 | 297.2 | 1019.5 | 5567. | 1316.4 | 269.7 | 390.9 | 36.0 | 369.5 | 70.8 | 363.9 | 114.2 |
| | % | 68.61 | 14.84 | 46.27 | 25.26 | 64.02 | 13.12 | 66.81 | 6.15 | 62.94 | 12.05 | 58.26 | 18.28 |
| Washings | μg | 394.5 | 190.9 | 540.1 | 181.5 | 539.8 | 230.6 | 52.8 | 20.5 | 80.0 | 41.1 | 136.5 | 73.2 |
| | % | 19.69 | 9.53 | 24.51 | 8.24 | 26.25 | 11.21 | 9.03 | 3.50 | 13.63 | 7.00 | 21.85 | 11.72 |
| Mass | μg | 1889.5 | 125.9 | 1647.1 | 594.1 | 1986.1 | 90.1 | 483.5 | 40.4 | 491.7 | 30.9 | 559.7 | 90.5 |
| balance | % | 94.32 | 6.28 | 74.74 | 26.96 | 96.60 | 4.38 | 82.64 | 6.90 | 83.75 | 5.27 | 89.61 | 14.49 |

From the data obtained in this series of experiments, it can be concluded that among the six formulations studied, the medical articles including a hydrogel that had been loaded with a 2% (by weight) caffeine solution with a pH value of 9.0 deliver caffeine most efficiently.

c. Influence of Hydrogel Thickness

To assess the influence of the thickness of a hydrogel on the efficiency of a hydrogel-containing medical article of the invention to deliver caffeine, hydrogel samples, prepared according to the method described in Example 7, but having a thickness of 1.45 mm, 2.9 mm, and 4.35 mm, were loaded with 0.5 wt. % and 2 wt. % caffeine solutions. Each hydrogel sample was applied to a Franz-type diffusion cell containing a porcine skin sample as described in Part B above. Receptor medium was totally removed and replaced at 2 hours, 4 hours, 6 hours, and 8 hours. The removed receptor medium was assayed to determine the amount of caffeine that was delivered to the receptor cell at a given time. Caffeine was extracted from the various other compartments of the cells at the end of the 24-hour test period. This experiment was conducted under both occlusive and non-occlusive conditions.

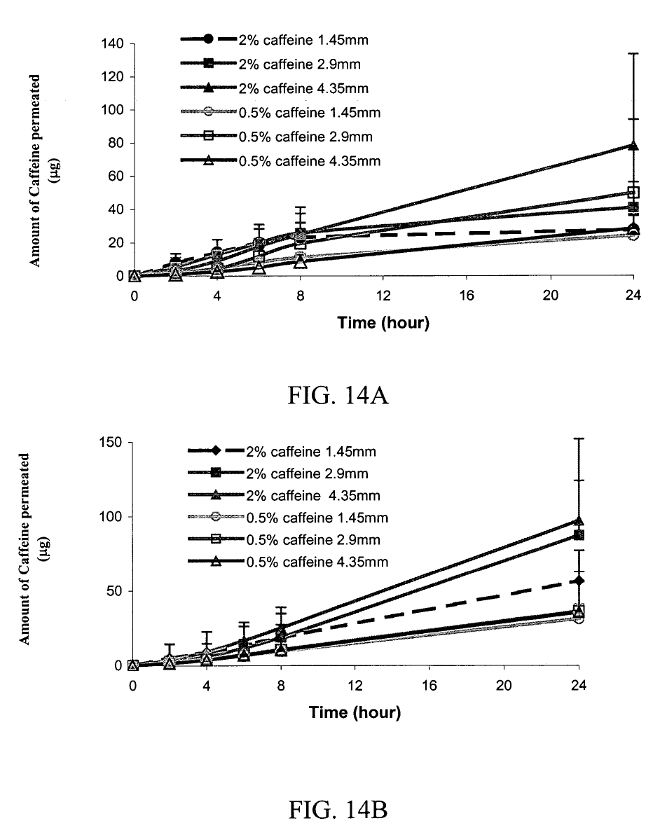

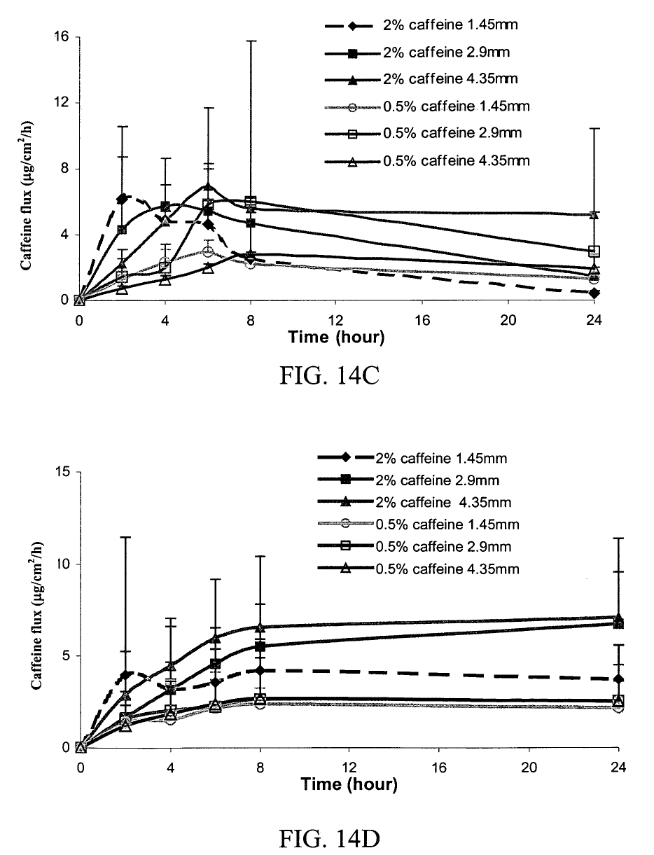

Table 22 summarizes the cumulative amount of caffeine that was recovered in the different compartments at the end of the 24-hour test period under the different experimental conditions. For each experimental condition, the experiment was conducted on at least 5 samples to obtain the average value presented in Table 22. FIGS. 14A-14D represent the corresponding caffeine permeation profiles versus time. FIGS. 14A and 14B show the cumulative amounts of caffeine permeated across the porcine skin samples (i.e., recovered from the receptor medium) over 24 hours under non-occlusive (FIG. 14A) and occlusive (FIG. 14B) conditions, respectively. FIGS. 14C and 14D show the flux of caffeine (calculated as the amount of caffeine permeated across the area of porcine skin per hour in μg/cm²/h) as a function of time under non-occlusive (FIG. 14C) and occlusive conditions (FIG. 14D), respectively.

Results

As shown in Table 22 below, under both occlusive and non-occlusive conditions, and regardless of the formulation applied, most of the caffeine remained either on the skin surface (as indicated in the amount that was recovered from the washings) or within the hydrogel. Moreover, it was observed that very little caffeine was absorbed by the epidermis and the dermis.

Referring to FIG. 14A, it was observed that, for both caffeine concentrations tested under non-occlusive conditions, the cumulative amount of caffeine that was delivered across the porcine skin during the first eight hours of the study was not statistically different (p>0.05) among the three different thicknesses. At the end of the 24-hour period, the cumulative amount of caffeine that permeated across the skin seemed to increase with the thickness of the hydrogel for the medical articles that had been loaded with 2% caffeine (by weight). However, because of large variability, no significant difference was observed between the different formulations. Furthermore, no significant difference was observed among the medical articles that had been loaded with 0.5% caffeine (by weight).

Referring to FIG. 14C, the flux profiles for the 2% caffeine group showed that as the thickness of the hydrogel increased, the flux of caffeine permeation across the skin became more sustained overtime. This could indicate that under non-occlusive conditions, thicker gels dehydrate more slowly, and, thus, they are able to maintain favorable diffusion conditions for a longer period of time.

TABLE 22
Influence of thickness on caffeine permeation profiles as released from hydrogel-containing medical articles according to the
invention. Each value represents the average cumulative amount of caffeine in μg (and % applied dose) recovered in the different
compartments at the end of the 24-hour test period. The average value presented was obtained from at least five samples.

| | | 2% caffeine | | 2% caffeine | | 2% caffeine | | 0.5% caffeine | | 0.5% caffeine | | 0.5% caffeine | |
|---|---|---|---|---|---|---|---|---|---|---|---|---|---|
| | | 1.45 mm | | 2.9 mm | | 4.35 mm | | 1.45 mm | | 2.9 mm | | 4.35 mm | |
| | | n = 5 | | n = 7 | | n = 6 | | n = 8 | | n = 8 | | n = 7 | |
| NON-OCCLUSIVE | | Avg. | ±Sd | Avg. | ±Sd | Avg. | ±Sd | Avg. | ±Sd | Avg. | ±Sd | Avg. | ±Sd |
| Receptor | μg | 27.35 | 9.41 | 40.83 | 15.30 | 77.99 | 55.13 | 24.06 | 16.60 | 49.48 | 44.24 | 28.26 | 7.81 |
| Fluid | % | 1.23 | 0.42 | 1.05 | 0.39 | 1.78 | 1.26 | 3.38 | 2.33 | 3.91 | 3.49 | 2.00 | 0.55 |
| Epidermis | μg | 3.56 | 1.51 | 3.10 | 1.81 | 8.93 | 7.61 | 0.81 | 0.63 | 6.64 | 10.12 | 4.89 | 5.42 |
| | % | 0.16 | 0.07 | 0.08 | 0.05 | 0.20 | 0.17 | 0.11 | 0.09 | 0.52 | 0.80 | 0.35 | 0.38 |
| Dermis | μg | 1.34 | 1.00 | 3.14 | 0.89 | 8.89 | 4.60 | 1.52 | 1.16 | 4.28 | 2.91 | 6.60 | 5.76 |
| | % | 0.06 | 0.05 | 0.08 | 0.02 | 0.20 | 0.10 | 0.21 | 0.16 | 0.34 | 0.23 | 0.47 | 0.41 |
| Hydrogel | μg | 1620.3 | 106.3 | 2292.6 | 71.3 | 2498.6 | 169.1 | 419.3 | 133.2 | 495.8 | 239.6 | 747.8 | 198.7 |
| | % | 72.78 | 4.77 | 59.06 | 1.84 | 56.88 | 3.85 | 58.97 | 18.73 | 39.14 | 18.91 | 52.92 | 14.07 |
| Washings | μg | 110.6 | 37.5 | 196.3 | 91.8 | 339.2 | 117.7 | 44.4 | 46.6 | 128.4 | 88.6 | 128.0 | 82.5 |
| | % | 4.97 | 1.69 | 5.06 | 2.36 | 7.72 | 2.68 | 6.25 | 6.56 | 10.14 | 6.99 | 9.06 | 5.84 |
| Mass | μg | 1763.2 | 120.1 | 2536.0 | 65.1 | 2933.7 | 66.7 | 490.1 | 78.8 | 684.6 | 156.3 | 915.6 | 128.2 |
| balance | % | 79.20 | 5.39 | 65.33 | 1.68 | 66.79 | 1.52 | 68.92 | 11.08 | 54.04 | 12.34 | 64.80 | 9.07 |

| | | | | | |
|---|---|---|---|---|---|
| 2% caffeine | 2% caffeine | 2% caffeine | 0.5% caffeine | 0.5% caffeine | 0.5% caffeine |
| 1.45 mm | 2.9 mm | 4.35 mm | 1.45 mm | 2.9 mm | 4.35 mm |
| n = 6 | n = 7 | n = 8 | n = 7 | n = 7 | n = 8 |

| OCCLUSIVE | | Avg | ±Sd | Avg | ±Sd | Avg | ±Sd | Avg | ±Sd | Avg | ±Sd | Avg | ±Sd |
|---|---|---|---|---|---|---|---|---|---|---|---|---|---|
| Receptor | μg | 56.53 | 20.57 | 87.32 | 36.67 | 97.12 | 54.86 | 31.33 | 9.62 | 36.42 | 26.48 | 35.73 | 20.27 |
| Fluid | % | 2.41 | 0.88 | 2.25 | 0.95 | 2.41 | 1.36 | 5.14 | 1.58 | 3.49 | 2.54 | 3.02 | 1.71 |
| Epidermis | μg | 14.47 | 5.61 | 23.57 | 8.55 | 18.36 | 12.18 | 5.53 | 1.99 | 8.43 | 4.14 | 7.67 | 3.98 |
| | % | 0.62 | 0.24 | 0.61 | 0.22 | 0.46 | 0.30 | 0.91 | 0.33 | 0.81 | 0.40 | 0.65 | 0.34 |
| Dermis | μg | 8.36 | 4.01 | 17.69 | 5.04 | 11.19 | 5.09 | 4.20 | 0.83 | 8.06 | 4.59 | 6.61 | 2.33 |
| | % | 0.36 | 0.17 | 0.46 | 0.13 | 0.28 | 0.13 | 0.69 | 0.14 | 0.77 | 0.44 | 0.56 | 0.20 |
| Hydrogel | μg | 1391.4 | 162.3 | 2113.9 | 170.4 | 2240.3 | 137.8 | 393.2 | 74.8 | 626.2 | 159.4 | 790.6 | 90.5 |
| | % | 59.32 | 6.92 | 54.55 | 4.40 | 55.69 | 3.42 | 64.52 | 12.27 | 59.96 | 15.26 | 66.83 | 7.65 |
| Washings | μg | 315.9 | 142.7 | 563.6 | 217.2 | 736.3 | 164.4 | 63.5 | 58.8 | 94.3 | 104.1 | 123.3 | 52.8 |
| | % | 13.47 | 6.08 | 14.54 | 5.61 | 18.30 | 4.09 | 10.42 | 9.64 | 9.03 | 9.97 | 10.43 | 4.46 |
| Mass | μg | 1786.7 | 114.6 | 2806.1 | 115.3 | 3103.3 | 143.3 | 497.8 | 26.6 | 773.4 | 68.0 | 963.9 | 75.3 |
| balance | % | 76.18 | 4.89 | 72.41 | 2.98 | 77.14 | 3.56 | 81.68 | 4.37 | 74.06 | 6.51 | 81.49 | 6.37 |

The results of this experiment suggested that under the experimental conditions used, the influence of the thickness of the hydrogel on caffeine permeation was minimal when the hydrogel was loaded with a 0.5% (by weight) caffeine solution. On the other hand, with respect to the 2% caffeine group, the amount of caffeine that was released and delivered across skin seemed to increase with gel thickness. However, because of the large variability in the data, no significant difference could be found between the various formulations in terms of their ability to deliver caffeine.

Results obtained under occlusive conditions were similar to those obtained under non-occlusive conditions. For both of the caffeine concentrations tested, the cumulative amount of caffeine that permeated across porcine skin after 8 and 24 hours was not statistically different (p>0.05) for the three different thicknesses tested (see FIGS. 14B and 14D).

From the data obtained in this experiment, it can be concluded that hydrogel thicknesses do not significantly affect how caffeine permeates across porcine skin over a 24-hour period under the experimental conditions used.

d. Influence of Protein Composition

To assess how the protein composition of a hydrogel may influence the efficiency of a hydrogel-containing medical article in delivering caffeine, hydrogel samples were prepared with six different types of proteins similar to the methods described in Examples 4 to 8. The hydrogel samples were then loaded with either a 2 wt. % or a 0.5 wt. % caffeine solution and applied to Franz-type diffusion cells containing porcine skin samples as described in Part B, Section 1, of this example, above. Receptor medium was totally removed and replaced at 2 hours, 4 hours, 6 hours, and 8 hours. The removed receptor medium was assayed to determine the amount of caffeine that was delivered to the receptor medium at a given time. Caffeine was extracted from the various compartments of the cells (i.e., hydrogel, receptor medium, epidermis, dermis, and washings) at the end of the 24-hour period. The six protein formulations tested in this study include hydrolyzed soy protein, native soy protein, bovine serum albumin, casein, pea albumin, and a casein/pea albumin mixture. The experiment was conducted under both occlusive and non-occlusive conditions. For the occlusive studies, only five protein formulations were tested (i.e., no data were obtained with regard to the pea albumin formulation).

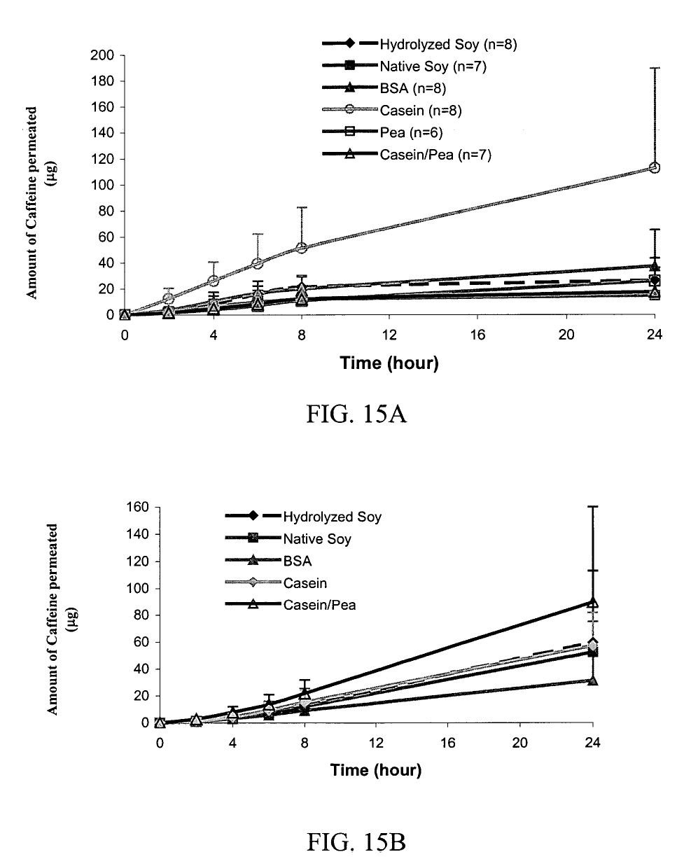

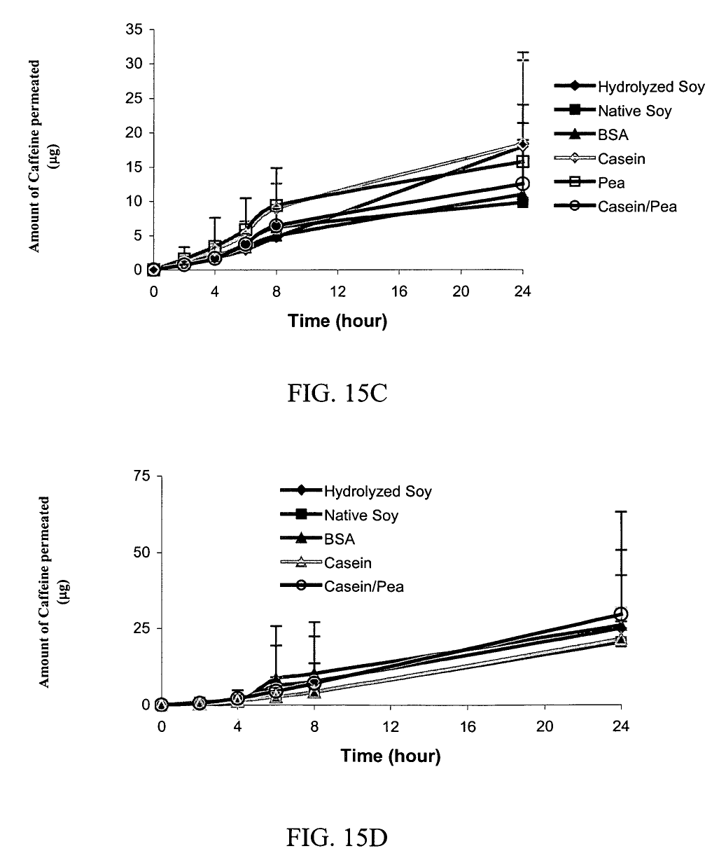

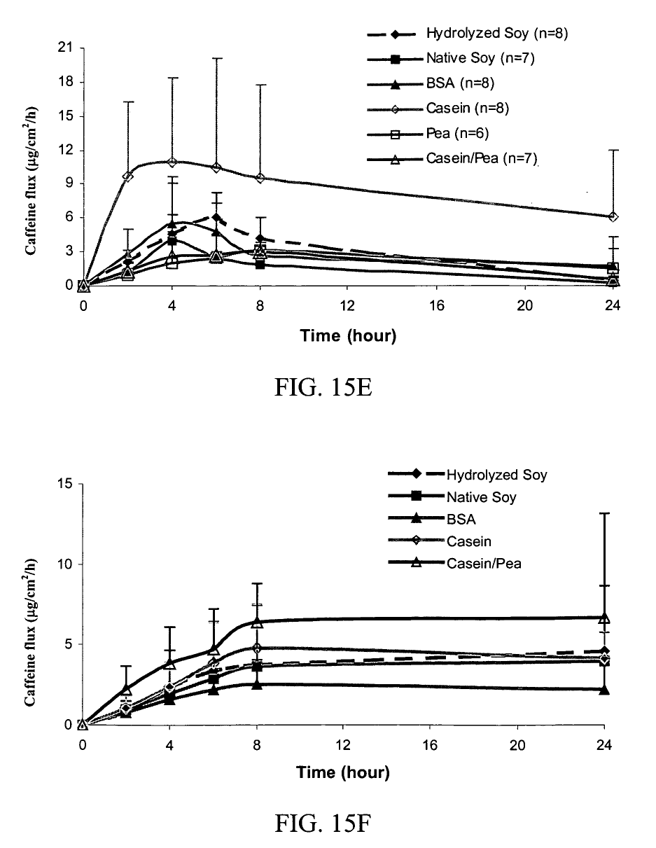

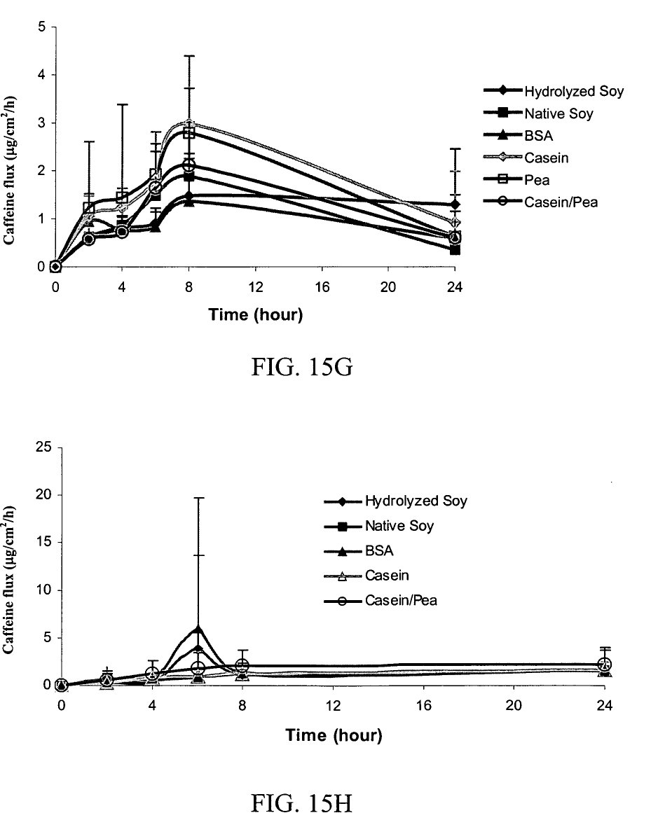

Tables 23 to 26 summarize the cumulative amount of caffeine that was recovered in the different compartments at the end of the 24-hour test period under the different experimental conditions. For each experimental condition, the experiment was conducted on at least 6 samples to obtain the average value presented in Tables 23 to 26. FIGS. 15A to 15H represent the corresponding caffeine permeation profiles versus time. FIGS. 15A to 15D show the cumulative amounts of caffeine permeated across the porcine skin samples (i.e., recovered from the receptor fluid) over a 24-hour period under non-occlusive (FIG. 15A, 2% formulations, and FIG. 15C, 0.5% formulations) and occlusive (FIG. 15B, 2% formulations, and FIG. 15D, 0.5% formulations) conditions. The data presented in FIGS. 15A to 15D are expressed in micrograms. FIGS. 15E to 15H show the flux of caffeine (calculated as the amount of caffeine permeated across the area of porcine skin per hour in μg/cm²/h) as a function of time under non-occlusive (FIG. 15E, 2% formulations, and FIG. 15G, 0.5% formulations) and occlusive (FIG. 15F, 2% formulations, and FIG. 15H, 0.5% formulations) conditions, respectively.

TABLE 23
Influence of protein composition on caffeine permeation profiles as released from hydrogel-containing medical
articles that had been loaded with a 2% (by weight) caffeine solution under non-occlusive conditions. Each
value represents the average cumulative amount of caffeine in μg (and % applied dose) recovered in the
different compartments at the end of the 24-hour test period as obtained from at least six samples.

| | | Hydrolyzed Soy | | Native Soy | | | | | | Pea | | Casein/Pea | |
|---|---|---|---|---|---|---|---|---|---|---|---|---|---|
| | | Protein | | Protein | | BSA | | Casein | | Albumin | | Albumin | |
| | | (n = 8) | | (n = 7) | | (n = 8) | | (n = 8) | | (n = 6) | | (n = 7) | |
| NON-OCCLUSIVE | | Avg | ±Sd | Avg | ±Sd | Avg | ±Sd | Avg | ±Sd | Avg | ±Sd | Avg | ±Sd |
| Receptor | μg | 26.43 | 9.77 | 14.67 | 12.22 | 37.28 | 28.25 | 112.55 | 76.59 | 25.90 | 17.62 | 17.52 | 8.43 |
| Fluid | % | 1.13 | 0.42 | 0.67 | 0.56 | 1.82 | 1.38 | 3.98 | 2.71 | 1.12 | 0.76 | 0.68 | 0.33 |
| Epidermis | μg | 3.38 | 1.65 | 6.12 | 4.64 | 4.33 | 5.78 | 9.65 | 5.95 | 10.70 | 6.20 | 3.93 | 2.41 |
| | % | 0.14 | 0.07 | 0.28 | 0.21 | 0.21 | 0.28 | 0.34 | 0.21 | 0.46 | 0.27 | 0.15 | 0.09 |
| Dermis | μg | 2.23 | 0.79 | 2.92 | 1.44 | 4.23 | 5.01 | 9.34 | 7.98 | 4.80 | 2.55 | 3.07 | 2.37 |
| | % | 0.10 | 0.03 | 0.13 | 0.07 | 0.21 | 0.25 | 0.33 | 0.28 | 0.21 | 0.11 | 0.12 | 0.09 |
| Hydrogel | μg | 2040.8 | 85.1 | 1901.6 | 73.5 | 1621.7 | 202.2 | 1613.8 | 384.8 | 1520.7 | 675.0 | 2184.6 | 60.1 |
| | % | 86.90 | 3.62 | 87.23 | 3.37 | 79.27 | 9.88 | 57.09 | 13.61 | 65.71 | 29.17 | 84.99 | 2.34 |
| Washings | μg | 153.9 | 30.8 | 94.2 | 62.6 | 192.6 | 173.7 | 365.6 | 210.1 | 328.1 | 427.6 | 80.2 | 41.2 |
| | % | 6.55 | 1.31 | 4.32 | 2.87 | 9.41 | 8.49 | 12.93 | 7.43 | 14.18 | 18.48 | 3.12 | 1.60 |
| Mass | μg | 2226.7 | 70.7 | 2019.5 | 29.0 | 1860.1 | 64.3 | 2110.9 | 137.5 | 1890.3 | 228.8 | 2289.3 | 50.2 |
| balance | % | 94.82 | 3.01 | 92.63 | 1.33 | 90.92 | 3.14 | 74.68 | 4.86 | 81.68 | 9.89 | 89.07 | 1.95 |

TABLE 24
Influence of protein composition on caffeine permeation profiles as released from hydrogel-containing medical
articles that had been loaded with a 2% (by weight) caffeine solution under occlusive conditions. Each value
represents the average cumulative amount of caffeine in μg (and % applied dose) recovered in the different
compartments at the end of the 24-hour test period as obtained from at least six samples.

| | | Hydrolyzed Soy | | Native Soy | | | | | | Casein/Pea | |
|---|---|---|---|---|---|---|---|---|---|---|---|
| | | Protein | | Protein | | BSA | | Casein | | Albumin | |
| OCCLUSIVE | | Avg | ±Sd | Avg | ±Sd | Avg | ±Sd | Avg | ±Sd | Avg | ±Sd |
| Receptor | μg | 59.46 | 53.46 | 52.17 | 22.96 | 31.30 | 19.86 | 57.21 | 24.54 | 89.46 | 70.66 |
| Fluid | % | 2.48 | 2.23 | 3.08 | 1.36 | 1.92 | 1.22 | 3.03 | 1.30 | 4.89 | 3.87 |
| Epidermis | μg | 15.91 | 8.07 | 17.58 | 8.38 | 11.79 | 8.02 | 13.37 | 2.49 | 13.32 | 9.76 |
| | % | 0.66 | 0.34 | 1.04 | 0.50 | 0.72 | 0.49 | 0.71 | 0.13 | 0.73 | 0.53 |
| Dermis | μg | 7.33 | 1.42 | 10.38 | 5.91 | 8.79 | 6.72 | 7.68 | 2.20 | 9.44 | 4.41 |
| | % | 0.31 | 0.06 | 0.61 | 0.35 | 0.54 | 0.41 | 0.41 | 0.12 | 0.52 | 0.24 |
| Hydrogel | μg | 1995.3 | 302.2 | 1606.3 | 262.7 | 1139.5 | 467.3 | 1425.7 | 164.4 | 1232.7 | 570. |
| | % | 83.38 | 12.63 | 94.95 | 15.53 | 69.89 | 28.66 | 75.52 | 8.71 | 67.42 | 30.95 |
| Washings | μg | 429.9 | 183.1 | 380.2 | 159.4 | 488.9 | 307.7 | 461.0 | 226.9 | 663.9 | 200. |
| | % | 17.96 | 7.65 | 22.47 | 9.42 | 29.99 | 18.87 | 24.42 | 12.02 | 36.31 | 11.12 |
| Mass | μg | 2507.9 | 203.2 | 2066.6 | 156.5 | 1680.3 | 217.2 | 1965.0 | 150.2 | 2008.8 | 340. |
| balance | % | 104.80 | 8.49 | 122.16 | 9.25 | 103.05 | 13.32 | 104.09 | 7.96 | 109.88 | 18.41 |

TABLE 25
Influence of protein composition on caffeine permeation profiles as released from hydrogel-containing medical
articles that had been loaded with a 0.5% (by weight) caffeine solution under non-occlusive conditions. Each
value represents the average cumulative amount of caffeine in μg (and % applied dose) recovered in the
different compartments at the end of the 24-hour test period as obtained from at least six samples.

| | | Hydrolyzed | | Native Soy | | | | | | Pea | | Casein/Pea | |
|---|---|---|---|---|---|---|---|---|---|---|---|---|---|
| NON- | | Soy Protein | | Protein | | BSA | | Casein | | Albumin | | Albumin | |
| OCCLUSIVE | | Avg | ±Sd | Avg | ±Sd | Avg | ±Sd | Avg | ±Sd | Avg | ±Sd | Avg | ±Sd |
| Receptor | μg | 17.92 | 12.49 | 9.76 | 5.16 | 10.97 | 10.37 | 18.27 | 13.26 | 15.74 | 8.25 | 12.51 | 6.36 |
| Fluid | % | 2.65 | 1.85 | 1.52 | 0.80 | 1.92 | 1.81 | 2.59 | 1.88 | 2.31 | 1.21 | 1.93 | 0.98 |
| Epidermis | μg | 4.41 | 3.12 | 2.36 | 1.65 | 2.88 | 2.51 | 2.00 | 0.75 | 2.70 | 0.65 | 2.13 | 0.53 |
| | % | 0.65 | 0.46 | 0.37 | 0.26 | 0.50 | 0.44 | 0.28 | 0.11 | 0.40 | 0.10 | 0.33 | 0.08 |
| Dermis | μg | 1.67 | 1.33 | 0.88 | 0.21 | 1.83 | 0.94 | 1.47 | 0.58 | 3.21 | 4.66 | 1.04 | 0.21 |
| | % | 0.25 | 0.20 | 0.14 | 0.03 | 0.32 | 0.16 | 0.21 | 0.08 | 0.47 | 0.68 | 0.16 | 0.03 |
| Hydrogel | μg | 411.2 | 124.5 | 461.6 | 37.5 | 260.8 | 40.4 | 305.3 | 30.2 | 548.9 | 20.1 | 290.6 | 23.5 |
| | % | 60.91 | 18.44 | 71.88 | 5.84 | 45.57 | 7.07 | 43.19 | 4.28 | 80.60 | 2.95 | 44.88 | 3.63 |
| Washings | μg | 36.1 | 33.8 | 15.0 | 10.1 | 28.6 | 15.4 | 26.5 | 9.0 | 32.8 | 10.3 | 20.3 | 6.4 |
| | % | 5.34 | 5.00 | 2.34 | 1.58 | 5.00 | 2.69 | 3.74 | 1.28 | 4.82 | 1.51 | 3.13 | 0.99 |
| Mass | μg | 471.2 | 87.1 | 489.6 | 24.5 | 305.0 | 15.8 | 353.5 | 22.8 | 603.4 | 17.5 | 326.6 | 15.0 |
| balance | % | 69.81 | 12.90 | 76.25 | 3.82 | 53.31 | 2.76 | 50.01 | 3.23 | 88.60 | 2.58 | 50.43 | 2.31 |

TABLE 26
Influence of protein composition on caffeine permeation profiles as released from hydrogel-containing
medical articles that had been loaded with a 0.5% (by weight) caffeine solution under
occlusive conditions. Each value represents the average cumulative amount of caffeine
in μg (and % applied dose) recovered in the different compartments at the end of
the 24-hour test period as obtained from at least six samples.

| | | Hydrolyzed | | Native Soy | | | | | | | |
|---|---|---|---|---|---|---|---|---|---|---|---|
| | | Soy Protein | | Protein | | BSA | | Casein | | Casein/Pea | |
| | | (u = 8) | | (n = 8) | | (n = 7) | | (n = 8) | | (n = 8) | |
| OCCLUSIVE | | Avg | ±Sd | Avg | ±Sd | Avg | ±Sd | Avg | ±Sd | Avg | ±Sd |
| Receptor | μg | 24.97 | 38.28 | 20.57 | 6.64 | 26.07 | 16.31 | 21.59 | 10.17 | 29.54 | 21.31 |
| Fluid | % | 4.06 | 6.22 | 5.36 | 1.73 | 6.18 | 3.86 | 4.71 | 2.22 | 6.54 | 4.72 |
| Epidermis | μg | 4.46 | 1.99 | 4.07 | 2.49 | 5.07 | 2.09 | 6.29 | 2.27 | 5.27 | 1.27 |
| | % | 0.73 | 0.32 | 1.06 | 0.65 | 1.20 | 0.50 | 1.37 | 0.49 | 1.17 | 0.28 |
| Dermis | μg | 2.30 | 1.45 | 3.55 | 1.71 | 2.29 | 1.77 | 3.45 | 0.87 | 3.39 | 0.88 |
| | % | 0.37 | 0.24 | 0.93 | 0.44 | 0.54 | 0.42 | 0.75 | 0.19 | 0.75 | 0.19 |
| Hydrogel | μg | 536.9 | 164.4 | 395.0 | 66.7 | 399.0 | 50.8 | 436.5 | 44.3 | 444.7 | 57.9 |
| | % | 87.27 | 26.73 | 102.91 | 17.37 | 94.51 | 12.02 | 95.28 | 9.66 | 98.46 | 12.83 |
| Washings | μg | 124.3 | 52.7 | 68.9 | 52.0 | 64.7 | 38.0 | 71.6 | 46.2 | 52.3 | 43.7 |
| | % | 20.20 | 8.57 | 17.94 | 13.55 | 15.32 | 9.01 | 15.63 | 10.08 | 11.58 | 9.68 |
| Mass | μg | 692.9 | 86.2 | 492.1 | 31.2 | 497.1 | 14.8 | 539.5 | 17.1 | 535.2 | 25.2 |
| balance | % | 112.63 | 14.01 | 128.20 | 8.12 | 117.75 | 3.50 | 117.75 | 3.73 | 118.50 | 5.57 |

Results

As shown in Tables 23 to 26, under both non-occlusive and occlusive conditions, and regardless of the formulation applied, most of the caffeine remained either on the skin surface (as indicated in the amount of caffeine recovered from the washings) or within the hydrogel. Further, it was observed that very little caffeine actually was absorbed in the epidermis or the dermis.

Referring to FIGS. 15A and 15E, it was observed that the casein formulation was the most effective in percutaneously delivering caffeine among the six formulations that had been loaded with a 2 wt. % caffeine solution and tested under non-occlusive conditions. However, it was also observed that hydrogels prepared with casein were soft and fragile. Because of their mechanical limitations, these casein-containing hydrogels were excluded from the discussion below.

With continued reference to FIGS. 15A and 15E, although no statistical difference was found between the different medical articles tested that had been loaded with a 2 wt. % caffeine solution, the bovine serum albumin (BSA) and pea albumin formulations exhibited a sustained release of the second highest amount of caffeine (after the casein formulation) over the duration of the experiment. Without being bound by the theory, it is believed that hydrogels prepared with BSA and pea albumin may be more resistant to dehydration and therefore were able to maintain favorable conditions for the delivery of caffeine across porcine skin over the course of the experiments, as compared to the other formulations that had dried up more rapidly.

Referring to FIGS. 15B and 15F, it was observed that the casein/pea albumin mixture formulation was the most effective in percutaneously delivering caffeine among the five formulations that had been loaded with a 2 wt. % caffeine solution and tested under occlusive conditions. No significant difference was found between the soy (hydrolyzed or native) and the casein formulations with regard to their effectiveness in delivering caffeine across porcine skin. It was further observed that the caffeine fluxes stabilized after 8 hours regardless of which type(s) of protein was used to prepare the hydrogels. At the end of the 24-hour period, no significant difference was observed among the different formulations with respect to the cumulative amount of caffeine that was delivered across porcine skin, with perhaps the exception of the BSA formulation, which, under these experimental conditions, seemed to have delivered significantly less drug (p<0.05) than the casein/pea albumin mixture and casein formulations.

Thus, under occlusive conditions, it was found that in the case of the hydrogel-containing medical articles of the invention that had been loaded with a 2% caffeine solution, the type(s) of protein used to prepare the hydrogels may significantly affect the physical properties of the hydrogels, as observed with the casein and BSA formulations. Nevertheless, because of the large variability in the amount of drug permeated across the skin within each group, no significant difference could be found between the different formulations tested.

Referring to FIGS. 15C and 15G, the kinetic profiles therein showed that in most cases the caffeine flux increased within the first 8 hours then decreased to reach a minimum at the 24-hour time point. One exception to this observation is the hydrolyzed soy formulations for which caffeine delivery was sustained between the eighth and twenty-fourth hours. It was also observed that, under occlusive condition, sustained delivery of caffeine was achieved by each of the five formulations over a 24-hour period.

Therefore, under both non-occlusive and occlusive conditions, it was shown that the type(s) of protein used to prepare the hydrogels included in the medical article embodiments tested in this experiment did not have any significant influence on the caffeine delivery profiles of the medical articles.

e. Influence of Application Time

To assess the influence of application time on caffeine delivery by hydrogel-containing medical articles according to the invention, hydrogel samples were prepared according to the method described in Example 7 above, and loaded with 2% and 0.5% (by weight) caffeine (SigmaUltra grade from Sigma Aldrich Chemical Co., Milwaukee, Wis.) solutions. The medical articles including the loading hydrogels were applied under non-occlusive and occlusive condition to Franz-type diffusion cells containing porcine skin samples as described in Section B, Part 1, of this example, above. Receptor medium was removed after 30 minutes and assayed. In a second set of experiments, receptor medium was removed and assayed at 30 minutes and 1 hour, and caffeine was extracted from the various compartments of the cells (i.e., hydrogel, washings, epidermis, dermis, and receptor medium) at the end of the 1-hour test period. Each set of experiments was carried out in duplicates.

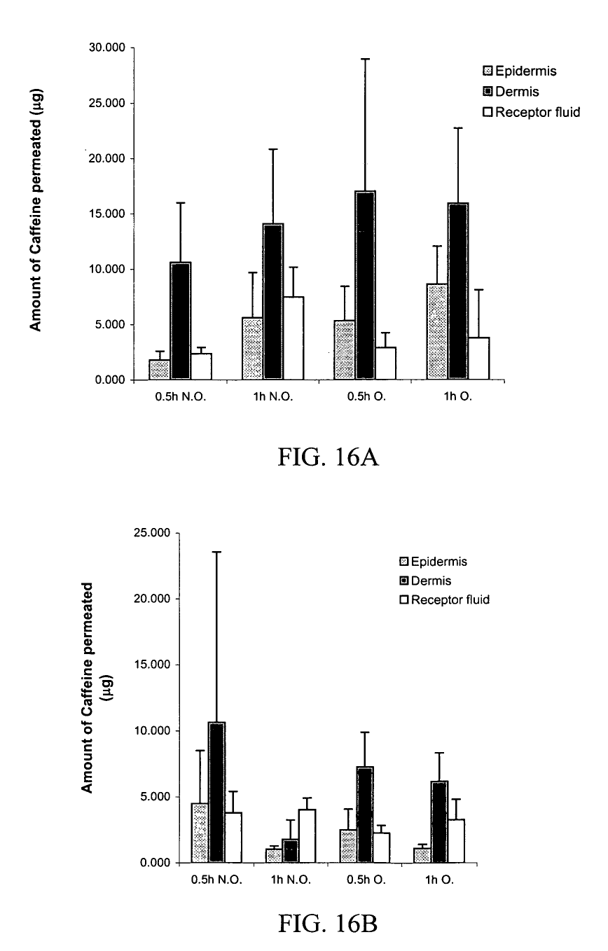

Results are summarized in Table 27 below and graphically presented in FIGS. 16A and 16B. FIGS. 16A and 16B show the total amount of caffeine that was recovered in the epidermis, the dermis, and the receptor fluid, at 30 minutes and 1 hour under both non-occlusive and occlusive conditions for the 2% (FIG. 16A) and 0.5% (FIG. 16B) caffeine formulations, respectively. Table 27 summarizes the cumulative amounts of caffeine that were recovered in the different compartments at the end of the 30-minute and 1-hour periods under the different experimental conditions. For each experimental condition, the experiment was conducted on at least 5 samples to obtain the average values presented in Table 27.

Results

As shown in FIGS. 16A and 16B, caffeine was readily released from the fully hydrated hydrogel-containing medical articles tested, regardless of their drug loading, under both non-occlusive and occlusive conditions over a 1-hour period. Transdermal delivery of caffeine was observed as early as 30 minutes after the medical articles had been applied, confirming that the medical articles of the invention are good candidates for short-term delivery of caffeine.

Additionally, when the 2% caffeine formulation was applied under non-occlusive conditions, there was no statistical difference (p>0.05) between the amount of caffeine that permeated across the skin (i.e., into the receptor fluid) after 30 minutes of application regardless of the total exposure time. Additionally, no significant difference was observed between the amount of caffeine that penetrated into and resided in the epidermis and the amount found in the dermis.

Similar results were observed with the 0.5% caffeine formulations applied under the same conditions. No statistical difference (p>0.05) was observed between the amount of caffeine that permeated across the skin (i.e., into the receptor fluid) after 30 minutes of application regardless of the total exposure time. However, it was observed that a higher amount of caffeine (p<0.05) permeated into the receptor medium at 30 minutes when the cell was treated for only 30 minutes than when the cell was treated for an hour. There were no significant difference (p>0.05) in the amount of caffeine recovered from the epidermis, dermis, and receptor fluid when the medical articles were applied under occlusion.

TABLE 27
Influence of application time on caffeine permeation profiles as released from hydrogel-containing medical articles
according to the invention. Each value represents the average cumulative amount of caffeine in μg (and % applied
dose) recovered in the different compartments at the end of the test period as obtained from at least six samples.

| Experimental | | RECEPTOR | | | | | | MASS |
|---|---|---|---|---|---|---|---|---|
| Conditions | | 30 min. | 1 hour | EPIDERMIS | DERMIS | WASHING | HYDROGEL | BALANCE |
| 2% Caffeine | μg | 2.38 ± 0.57 | — | 1.81 ± 0.79 | 10.62 ± 5.38 | 50.30 ± 20.95 | 3204.15 ± 143.70 | 3269.25 ± 145.86 |
| Unoccluded | % | 0.08 ± 0.02 | — | 0.06 ± 0.03 | 0.34 ± 0.17 | 1.62 ± 0.67 | 103.04 ± 4.62 | 105.13 ± 4.69 |
| n = 7 | | | | | | | | |
| 2% Caffeine | μg | 2.98 ± 1.32 | 7.50 ± 2.67 | 5.63 ± 4.06 | 14.08 ± 6.77 | 47.84 ± 13.74 | 3175.38 ± 51.01 | 3250.42 ± 60.32 |
| Unoccluded | % | 0.10 ± 0.04 | 0.24 ± 0.09 | 0.18 ± 0.13 | 0.45 ± 0.22 | 1.54 ± 0.44 | 102.11 ± 1.64 | 104.53 ± 1.94 |
| n = 7 | | | | | | | | |
| 2% Caffeine | μg | 2.90 ± 1.35 | — | 5.33 ± 3.09 | 17.01 ± 11.89 | 89.40 ± 91.06 | 3134.19 ± 351.63 | 3248.83 ± 266.60 |
| Occluded | % | 0.09 ± 0.04 | — | 0.17 ± 0.10 | 0.55 ± 0.38 | 2.87 ± 2.93 | 100.79 ± 11.31 | 104.48 ± 8.57 |
| n = 8 | | | | | | | | |
| 2% Caffeine | μg | 4.83 ± 2.59 | 10.14 ± 4.33 | 8.61 ± 3.41 | 15.90 ± 6.78 | 84.06 ± 45.54 | 3112.73 ± 164.19 | 3231.44 ± 163.33 |
| Occluded | % | 0.16 ± 0.08 | 0.33 ± 0.14 | 0.28 ± 0.11 | 0.51 ± 0.22 | 2.70 ± 1.46 | 100.10 ± 5.28 | 103.92 ± 5.25 |
| n = 5 | | | | | | | | |
| 0.5% Caffeine | μg | 3.78 ± 1.63 | — | 4.49 ± 4.03 | 10.66 ± 12.91 | 7.67 ± 0.04 | 804.59 ± 70.98 | 831.20 ± 67.37 |
| Unoccluded | % | 0.44 ± 0.19 | — | 0.52 ± 0.47 | 1.23 ± 1.49 | 0.89 ± 0.00 | 92.93 ± 8.20 | 96.00 ± 7.78 |
| n = 7 | | | | | | | | |
| 0.5% Caffeine | μg | 1.69 ± 0.59 | 4.02 ± 0.90 | 1.04 ± 0.23 | 1.78 ± 1.46 | 8.26 ± 1.12 | 789.23 ± 36.53 | 804.33 ± 36.08 |
| Unoccluded | % | 0.20 ± 0.07 | 0.46 ± 0.10 | 0.12 ± 0.03 | 0.21 ± 0.17 | 0.95 ± 0.13 | 91.15 ± 4.22 | 92.90 ± 4.17 |
| n = 7 | | | | | | | | |
| 0.5% Caffeine | μg | 2.26 ± 0.58 | — | 2.49 ± 1.57 | 7.28 ± 2.61 | 21.56 ± 24.36 | 753.69 ± 45.13 | 787.29 ± 29.87 |
| Occluded | % | 0.26 ± 0.07 | — | 0.29 ± 0.18 | 0.84 ± 0.30 | 2.49 ± 2.81 | 87.05 ± 5.21 | 90.93 ± 3.45 |
| n = 5 | | | | | | | | |
| 0.5% Caffeine | μg | 2.27 ± 0.70 | 3.27 ± 1.55 | 1.12 ± 0.30 | 6.18 ± 2.18 | 25.75 ± 6.64 | 810.41 ± 14.53 | 846.74 ± 9.90 |
| Occluded | % | 0.26 ± 0.08 | 0.38 ± 0.18 | 0.13 ± 0.04 | 0.71 ± 0.25 | 2.97 ± 0.77 | 93.60 ± 1.68 | 97.80 ± 1.14 |
| n = 7 | | | | | | | | |

For all of the analyzed compartments, there were no statistical difference (p>0.05) between the results obtained under non-occlusive conditions and those obtained under occlusive conditions, regardless of the concentration of caffeine inside the hydrogel or the duration of the application of the medical articles.

The data obtained in this experiment showed that caffeine was readily available for release when incorporated into hydrogel-containing medical articles of the invention, and its permeation across porcine skin was observed as early as 30 minutes after the medical article had been applied. Occlusion of the donor compartment did not seem to have a significant effect on the permeation profile of caffeine under the experimental conditions used.

2. Lidocaine Delivery Via Hydrogel-Containing Medical Articles

a. Influence of Drug Loading

Hydrogels prepared by the method described in Example 7 were soaked in the appropriate lidocaine solution (described below) for 1 hour at room temperature under gentle agitation. A second impregnation was performed in the same solution overnight. The lidocaine solutions, in addition to the amount of lidocaine described below, further contained EDTA (0.2 wt. %) and NaH₂PO₄ (0.16 wt. %). The loaded hydrogels were then cut into 9 mm-round pieces and kept in solution until their application onto porcine skin. The integration volume represented 10 times the volume of the dehydrated hydrogels. The hydrogels had a pH of 5.5.

After cleaning with cold tap water, porcine skin was shaved and then stored frozen in aluminum foil at −20° C. Before use, the skin was thawed and then dermatomed to a thickness of 510 μm with a Padgett Electro-Dermatome (Padgett Instrument Inc, Kansas City, Mo.). Percutaneous absorption was measured using 0.9 cm-diameter horizontal glass diffusion cells consisting of a donor (where the tested sample is applied) and a receptor (where a tested active might diffuse to) compartment (OECD guidelines, 2000). Such cells, known as Franz-type diffusion cells, or static cells, were supplied by Logan Instrument Corp (Somerset, N.J.). Dermatomed porcine skin samples were cut with surgical scissors and placed between the two halves of a diffusion cell, with stratum corneum facing the donor chamber. The area available for diffusion was 0.635 cm² and the receptor phase was 4.5 ml.

The receptor chamber was filled with 0.22 μm-filtered phosphate saline buffer (pH 7.4) containing 20% (v/v) ethanol and allowed to equilibrate to the needed temperature. Temperature of the skin surface was maintained at 37° C. throughout the experiment by placing diffusion cells into a dry block heater set to 37° C. The receptor compartment contents were continuously agitated by small PTFE-coated magnetic stirring bars.

Skin samples were allowed to equilibrate with receptor medium at 37° C. for at least one hour before application of test formulations. Groups were randomized, and hydrogel samples that had been loaded with 1 wt. %, 2 wt. %, and 5 wt. % lidocaine (SigmaUltra grade from Sigma-Aldrich Chemical Co., Milwaukee, Wis.) solution were applied to each individual cell under occlusive conditions for 24 hours. Receptor fluid was removed at predetermined times (2 hours, 4 hours, 6 hours and 8 hours) and replaced with fresh temperature-equilibrated buffer. The removed receptor fluid was assayed to determine the amount of lidocaine delivered to the receptor medium at a given time.

At the end of the experiment, the hydrogel-containing medical articles were removed from the skin surface and were placed in methanol for 48 hours at room temperature to allow lidocaine extraction. The donor cells were washed exhaustively with a methanol/water mixture (20/80; v/v). The exposed skin was excised, and the epidermis was separated from the dermis. The two skin strata respectively were placed in a methanol/water mixture (80/20; v/v) for 48 hours at room temperature. All samples (receptor medium, epidermis, dermis, hydrogels and washings) were assayed by high performance liquid chromatography (HPLC) for mass balance verification.

The parameters for the HPLC setup were as follows. The HPLC instrumentation consisted of an HP1050 quaternary solvent delivery system, a variable wavelength detector, a column, and an automated sample injector. The column (ACE 3 C4, 5.0 cm×4.6 mm i.d.) was used at room temperature. The flow rate was 1.5 ml/min, and the effluent was monitored at 254 nm. The run time was 3.5 minutes, and the injected volume was 25 μl.

The lidocaine concentration in each sample was determined, individually, against a 9-point linear calibration curve. Standard lidocaine solutions with concentrations of 5 μg/ml, 10 μg/ml, 50 μg/ml, 100 μg/ml, 500 μg/ml, 1000 μg/ml, 2500 μg/ml, 5000 μg/ml, and 7500 μg/ml were prepared by successive dilutions of a 10 mg/ml lidocaine stock solution with mobile phase. Each standard lidocaine solution was injected in triplicate.

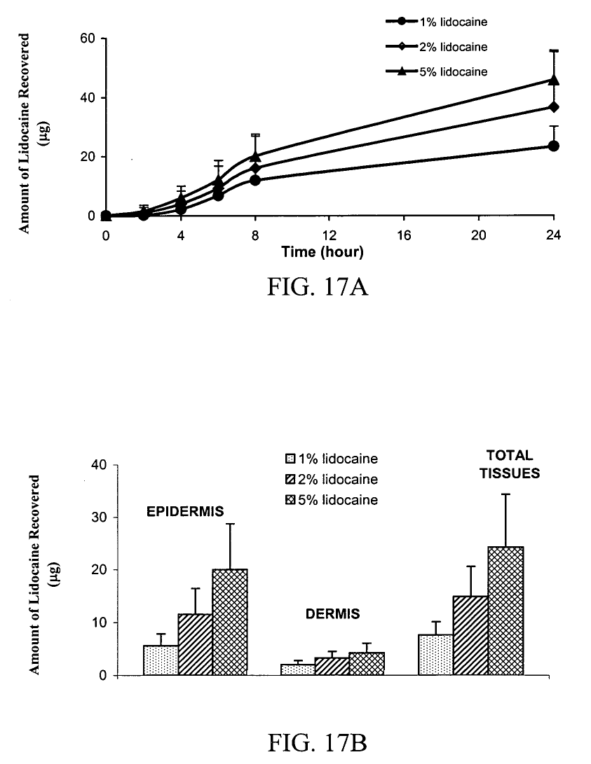

The chromatograms obtained were used to calculate the total cumulative amount of lidocaine recovered in each compartment (hydrogel, washing, epidermis, dermis, and receptor fluid). Results were presented in Table 28 and FIGS. 17A and 17B. FIG. 17A shows the total amount of lidocaine permeated across porcine skin over a 24-hour period for each of the three tested formulations. FIG. 17B shows the amount of lidocaine extracted from the epidermis and dermis, alone and combined, over a 24-hour period with respect to the same three formulations. Table 28 summarizes the cumulative amount of lidocaine that was recovered in each of the compartments at the end of the 24-hour period under the different experimental conditions. For each experimental condition, the experiment was conducted on eight samples to obtain the average value presented in Table 28.

Results

The data collected in this part of the study show that lidocaine was readily released from fully-hydrated hydrogel-containing medical articles of the invention at each of the concentrations tested under occlusive conditions within a 24-hour period. Thus, it was concluded that the medical articles of the invention did not represent a limiting factor for lidocaine delivery.

The data also showed that most of the lidocaine applied on the skin sample remained in the hydrogel as indicated in Table 28. Additionally, the amount of lidocaine that permeated across the skin (as indicated by the amount of lidocaine recovered from the receptor fluid) increased with increasing lidocaine concentrations. It was observed that with an increase in concentration of 1% to 5%, the dose-response curve obtained was not linear (R²=0.86), suggesting that lidocaine permeation rate decreases when drug concentration increases.

It was also observed that the amount of lidocaine recovered from the epidermis was much higher than the amount recovered from the dermis. This is expected as the target sites of lidocaine are located at the nerve ends in the basal epidermis. The epidermal retention of lidocaine appeared to be concentration-dependent, although the dose-response curve was also not linear.

It may be concluded from these results that drug loading seems to have an influence on the transdermal delivery and epidermal retention of lidocaine under the experimental conditions used.

TABLE 28
Influence of drug loading on lidocaine permeation profiles as released
from hydrogels according to the invention. Each value represents the average
cumulative amount of lidocaine in μg (and % applied dose) recovered
in the different compartments at the end of the 24-hour test period. The
average value presented was obtained from eight samples.

| | | 1% lidocaine | | 2% lidocaine | | 5% lidocaine | |
|---|---|---|---|---|---|---|---|
| | | Average | ±Sd | Average | ±Sd | Average | ±Sd |
| Receptor | Amt (μg) | 23.29 | 6.81 | 36.56 | 18.67 | 45.77 | 9.98 |
| Fluid | % Dose | 1.74 | 0.51 | 1.32 | 0.67 | 0.62 | 0.13 |
| Epidermis | Amt (μg) | 5.60 | 2.28 | 11.57 | 4.88 | 20.05 | 8.71 |
| | % Dose | 0.42 | 0.17 | 0.42 | 0.18 | 0.27 | 0.12 |
| Dermis | Amt (μg) | 2.04 | 0.75 | 3.30 | 1.22 | 4.24 | 1.83 |
| | % Dose | 0.15 | 0.06 | 0.12 | 0.04 | 0.06 | 0.02 |
| Hydrogel | Amt (μg) | 1218.7 | 107.7 | 2587.5 | 240.5 | 6493.6 | 430.7 |
| | % Dose | 91.13 | 8.05 | 93.40 | 8.68 | 87.48 | 5.80 |
| Washings | Amt (μg) | 59.7 | 34.8 | 114.5 | 38.2 | 355.6 | 22.64 |
| | % Dose | 4.46 | 2.61 | 4.13 | 1.38 | 4.79 | 3.05 |
| Mass | Amt (μg) | 1309.9 | 109.3 | 2753.4 | 251.7 | 6919.3 | 310.8 |
| Balance | % Dose | 97.91 | 8.17 | 99.38 | 9.08 | 93.21 | 4.19 |

b. Influence of pH

To assess the influence of the pH on lidocaine delivery via hydrogel-containing medical articles of the invention, hydrogel samples prepared according to the method described in Example 7 were loaded with lidocaine and buffered. Specifically, a first set of the medical articles tested in this experiment were loaded with a 1 wt. % lidocaine solution and buffered to adjust their pH to 3.0, 5.5, and 7.0. A second set of the medical articles were loaded with a 5 wt. % lidocaine solution and buffered to adjust their pH to 3.0 and 5.5. The lidocaine used in this experiment was SigmaUltra grade purchased from Sigma Aldrich Chemical Co. (Milwaukee, Wis.). The two sets of medical articles were applied to Franz-type diffusion cells containing porcine skin samples as described previously under occlusive condition for a 24-hour period. Receptor medium was removed at 2 hours, 4 hours, 6 hours and 8 hours and replaced with fresh temperature-equilibrated buffer. The removed receptor medium was assayed to determined the amount of lidocaine delivered to the receptor cell at a given time. Lidocaine was extracted from the various compartments of the cells (epidermis, dermis, washings, hydrogel, and receptor medium) at the end of the 24-hour test period.

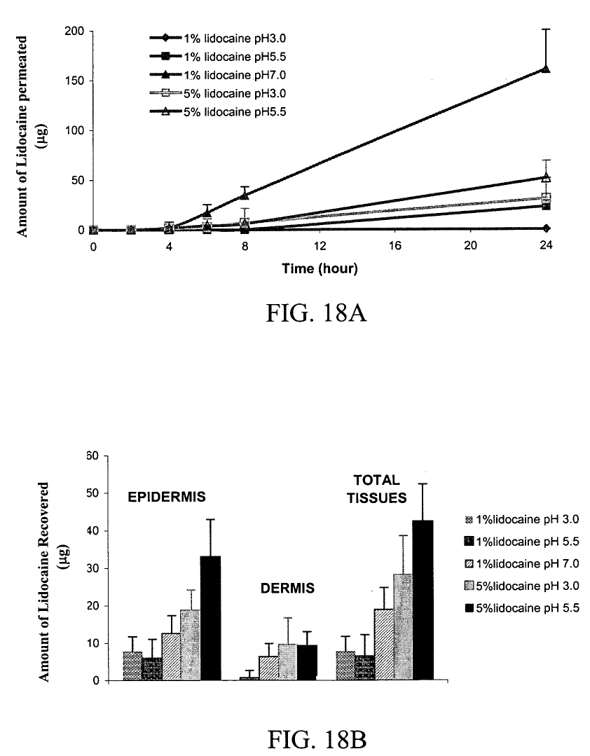

Results are presented in Table 29 and in FIGS. 18A and 18B. Table 29 summarizes the cumulative amounts of lidocaine that were recovered in the different compartments at the end of the 24-hour period under the different experimental conditions. For each experimental condition, the experiment was conducted on eight samples to obtain the average value presented in Table 29. FIG. 18A shows the cumulative amount of lidocaine permeated across porcine skin (i.e., recovered from the receptor medium) over a 24-hour period with regard to each of the five formulations tested. FIG. 18B shows the amount of lidocaine extracted from the epidermis and dermis, alone and combined, over a 24-hour period by the same five formulations.

Results

Results showed that, regardless of the formulation tested, most of the lidocaine applied on the skin remained in the hydrogel as indicated in Table 29. Additionally, as shown in FIG. 18A and Table 29, virtually no lidocaine was delivered by the 1% formulation with a pH of 3.0. With the 1% formulations, it was observed that the amount of lidocaine delivered across the skin significantly increased when the pH increased from 3.0 to 7.0.

With respect to the 5% formulations, delivery of lidocaine was observed with the formulation having a pH of 3.0, and the actual amount delivered was smaller than the formulation having a pH of 5.5. These observations are consistent with the results obtained with the 1% formulations.

Referring to FIG. 18B and Table 29, no lidocaine was recovered from the dermis at pH 3.0. This suggests that lidocaine was not transdermally delivered under this experimental condition. Increasing the pH from 3.0 to 7.0 led to a significant increase in the amount of lidocaine delivered to the dermis, indicating that transdermal delivery of lidocaine is possible and quite effective at pH 7.0 with a 1% formulation. When the 5% formulations were tested, dermal absorption of lidocaine was observed both at pH 3.0 and at pH 5.5; however, there was no significant difference between these two formulations in the amount of caffeine that was transdermally delivered.

From the data obtained, it can be concluded that among the five formulations tested, the 1% formulation with a pH of 7.0 was capable of the most efficient transdermal lidocaine delivery.

With continued reference to FIG. 18B, epidermal retention of lidocaine was observed in each of the five formulations tested. As mentioned in the description of the drug loading experiment above, receptors for lidocaine are present in the epidermis but not in the dermis. As such, lidocaine can only be retained in the epidermis, although the dermis may absorb a small amount of lidocaine. The data presented in Table 29 and in FIG. 18B are consistent with these known facts. In the case of the 1% formulations, the formulation with a pH of 7.0 exhibited the highest amount of lidocaine epidermal retention. An even larger amount of lidocaine was retained in the epidermis when the 5% formulations were applied. From the data obtained in this experiment, it can be concluded that among the five formulations tested, the largest amount of lidocaine was retained in the epidermis when the 5% formulation with a pH of 5.5 was applied.

The results from this experiment suggest that both the transdermal delivery and the epidermal retention of lidocaine may be pH-dependent.

TABLE 29
Influence of pH on lidocaine permeation profiles as released from hydrogel-containing
medical articles of the invention. Each value represents the average cumulative amount
of lidocaine in μg (and % applied dose) recovered in the different compartments
at the end of the 24-hour test period as obtained from eight samples.

| | | 1% lidocaine | | 1% lidocaine | | 1% lidocaine | | 5% lidocaine | | 5% lidocaine | |
|---|---|---|---|---|---|---|---|---|---|---|---|
| | | pH 3.0 | | pH 5.5 | | pH 7.0 | | pH 3.0 | | pH 5.5 | |
| | | Avg. | ±Sd | Avg. | ±Sd | Avg. | ±Sd | Avg. | ±Sd | Avg. | ±Sd |
| Receptor | μg | 0.00 | 0.00 | 22.76 | 5.79 | 160.27 | 39.73 | 30.69 | 37.80 | 51.19 | 27.10 |
| Fluid | % | 0.00 | 0.00 | 1.68 | 0.43 | 11.00 | 2.73 | 0.48 | 0.60 | 0.83 | 0.44 |
| Epidermis | μg | 7.81 | 3.93 | 5.79 | 5.27 | 12.60 | 4.82 | 18.67 | 5.56 | 33.09 | 9.89 |
| | % | 0.74 | 0.37 | 0.43 | 0.39 | 0.86 | 0.33 | 0.29 | 0.09 | 0.53 | 0.16 |
| Dermis | μg | 0.00 | 0.00 | 0.70 | 1.97 | 6.33 | 3.50 | 9.54 | 7.16 | 9.26 | 3.77 |
| | % | 0.00 | 0.00 | 0.05 | 0.15 | 0.43 | 0.24 | 0.15 | 0.11 | 0.15 | 0.06 |
| Hydrogel | μg | 739.57 | 134.25 | 944.06 | 97.36 | 916.25 | 69.23 | 4592.74 | 348.06 | 4673.28 | 670.13 |
| | % | 69.86 | 12.68 | 69.57 | 7.18 | 62.90 | 4.75 | 72.44 | 5.49 | 75.40 | 10.81 |
| Washings | μg | 225.42 | 111.09 | 256.39 | 81.97 | 193.20 | 61.83 | 809.71 | 400.07 | 944.86 | 379.93 |
| | % | 21.29 | 10.49 | 18.89 | 6.04 | 13.26 | 4.24 | 12.77 | 6.31 | 15.24 | 6.13 |
| Mass | μg | 972.81 | 79.34 | 1229.68 | 88.39 | 1288.64 | 118.96 | 5461.36 | 512.45 | 5711.68 | 435.32 |
| Balance | % | 91.89 | 7.49 | 90.62 | 6.51 | 88.46 | 8.17 | 86.13 | 8.08 | 92.15 | 7.02 |

c. Influence of Application Time

To assess the influence of application time on lidocaine delivery by hydrogel-containing medical articles according to the invention, hydrogel samples were prepared according to the method described in Example 7 above, and loaded with 1 wt. % and 2 wt. % lidocaine solutions and further buffered to obtain a pH of 3.0, 5.5, or 7.0. The medical articles were then applied to Franz-type diffusion cells containing porcine skin samples as described above for a 24-hour period under occlusive condition. Receptor medium was removed at a given time, and lidocaine was extracted from the various compartments of the cells at the end of the study. Four sets of experiments were conducted to evaluate the influence of application time on lidocaine delivery profiles. The four sets of experiments were carried out for 15 minutes, 30 minutes, 1 hour, and 2 hours, respectively.

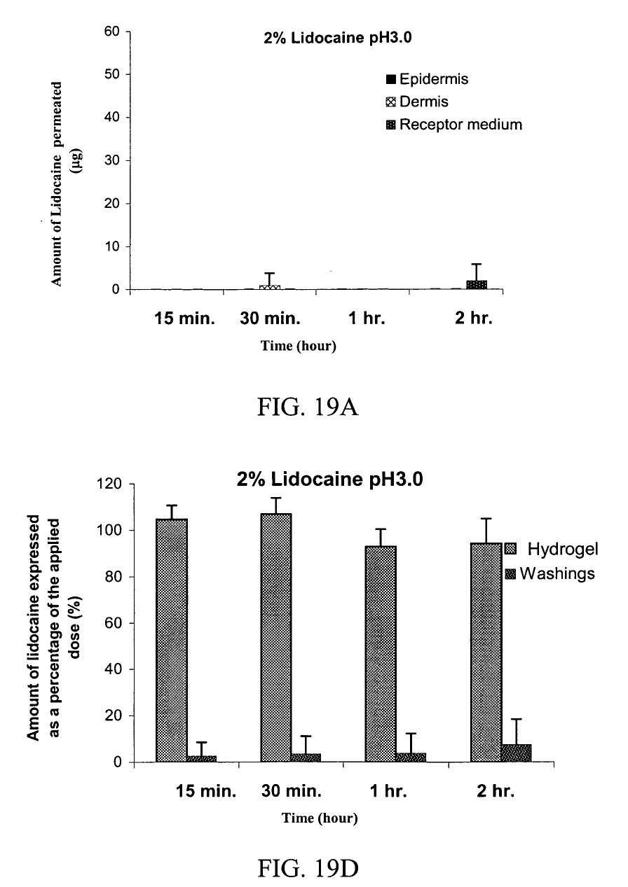

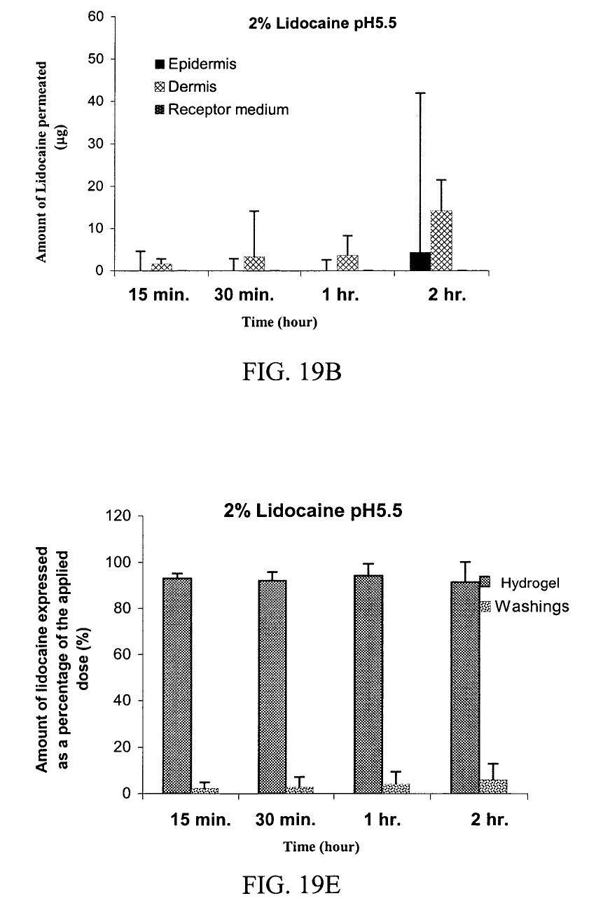

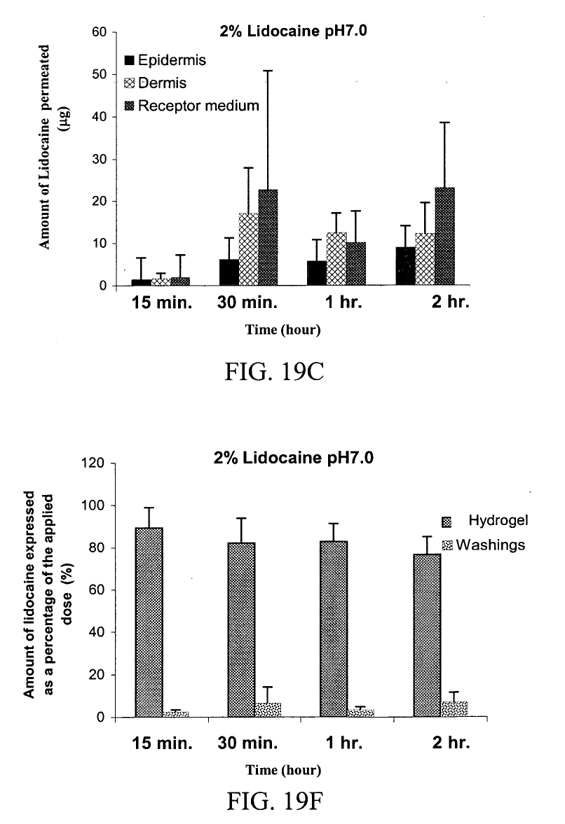

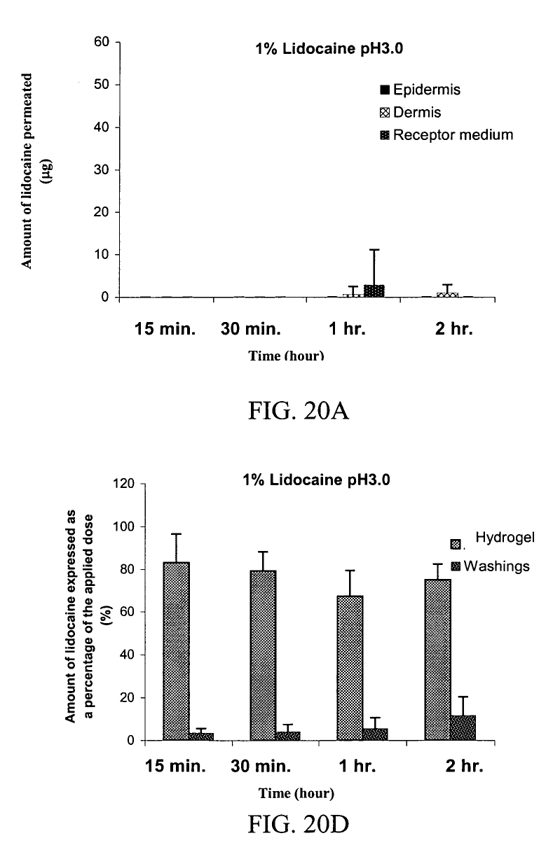

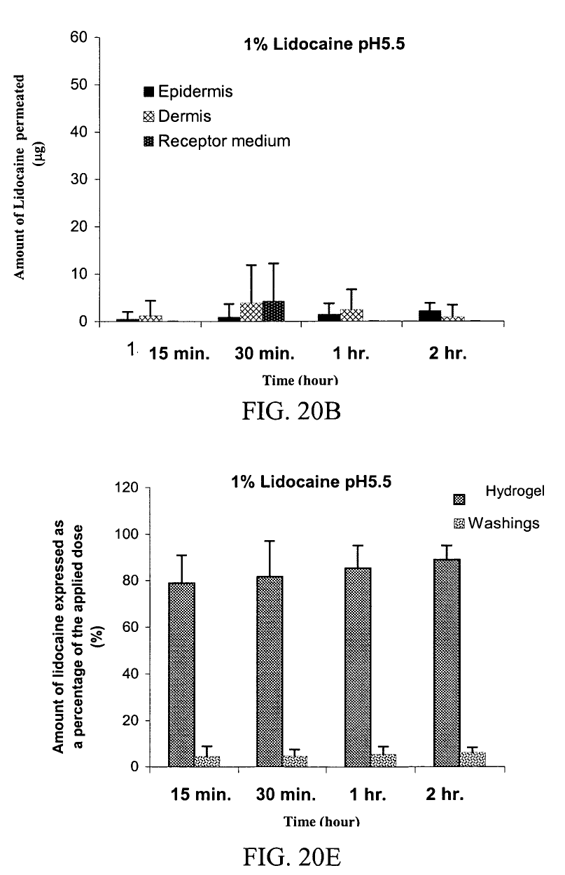

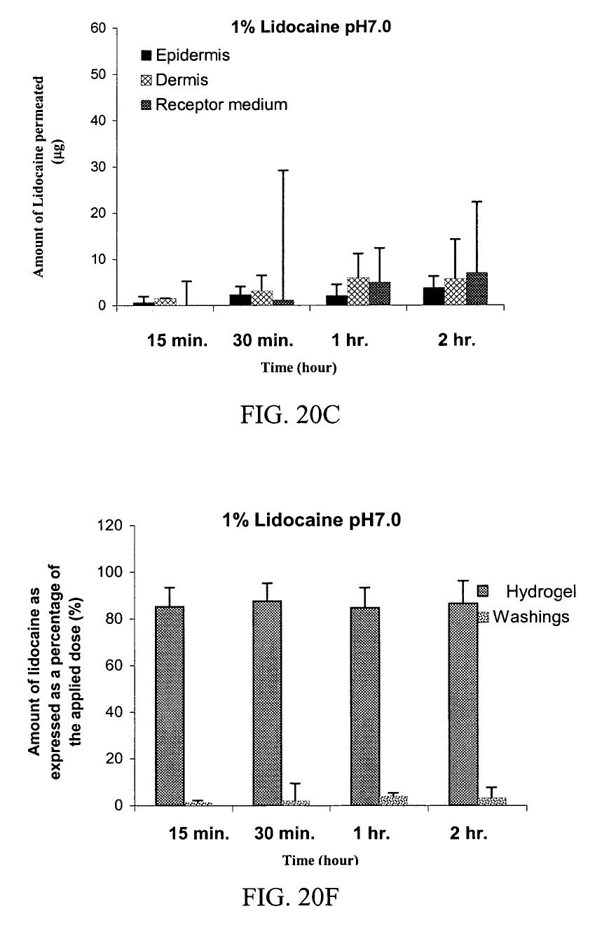

Results are summarized in Tables 30 to 33 and in FIGS. 19A to 19F and 20A to 20F. FIGS. 19A, 19B, and 19C show the amount of lidocaine (expressed in micrograms) released and delivered to the receptor cell, epidermis and dermis as a function of time by medical articles including hydrogels that had been loaded with a 2% lidocaine solution (by weight) buffered to a pH of 3.0 (FIG. 19A), 5.5 (FIG. 19B) and 7.0 (FIG. 19C), respectively. FIGS. 19D, 19E, 19F show the amount of lidocaine (expressed as a percentage of the applied dose) that was extracted from the hydrogels and the washings as a function of time, as delivered by medical articles including hydrogels that had been loaded with a 2% lidocaine solution (by weight) buffered to a pH of 3.0 (FIG. 19D), 5.5 (FIG. 19E) and 7.0 (FIG. 19F), respectively. FIGS. 20A, 20B, 20C show the amount of lidocaine (expressed in micrograms) released and delivered to the receptor cell, epidermis and dermis as a function of time, by medical articles including hydrogels that had been loaded with a 1% lidocaine solution (by weight) buffered to a pH of 3.0 (FIG. 20A), 5.5 (FIG. 20B) and 7.0 (FIG. 20C), respectively. FIGS. 20D, 20E, 20F show the amount of lidocaine (expressed as a percentage of the applied dose) that was extracted from the hydrogels and the washings as a function of time, as delivered by medical articles including hydrogels that had been loaded with a 1% lidocaine solution (by weight) buffered to a pH of 3.0 (FIG. 20D), 5.5 (FIG. 20E) and 7.0 (FIG. 20F), respectively. Tables 30 to 33 summarize the cumulative amount of lidocaine that was recovered in the different compartments with respect to the six formulations at the end of the 15-minute (Table 30), 30-minute (Table 31), 1-hour (Table 32) and 2-hour (Table 33) application periods, respectively. For each experimental condition, the experiment was conducted on eight samples to obtain the average values presented in Tables 30 to 33.

Results

As shown in Tables 30 to 33 and in FIGS. 19A to 10F and 20A to 20F, regardless of the formulations and the duration of the application, most of the lidocaine applied on the skin remained in the hydrogels and the washings. Moreover, lidocaine percutaneous absorption was observed to be dependent on both the drug loading and the pH of the hydrogel included in the medical articles, when the medical articles were applied for a short period of time (e.g., up to 2 hours).

Data presented in FIGS. 19A to 19F indicate that, with the 2% formulations having a pH of either 3.0 or 5.5, only a very limited amount of lidocaine was delivered across the skin. Increasing the pH to 7.0 was observed to have led to a significant increase in the amount of lidocaine recovered from the epidermis, the dermis and the receptor fluid. A small amount of lidocaine was detectable in the three compartments as soon as 15 minutes after application. Increasing the duration of the application also led to an increase in the amount of lidocaine that permeated across the skin. From the data obtained, and as best shown in FIGS. 19A to 19C, it was observed that lidocaine was not epidermally retained when the application period was 2 hours or less, since the amount of lidocaine recovered from the dermis was greater than the amount recovered from the epidermis under these experimental conditions.

Data presented in FIGS. 20A to 20F indicate that, in the case of the 1% formulations, no delivery of lidocaine was observed at pH 3.0 and 5.5. It was only at a pH of 7.0 that drug permeation and absorption were observed. It was further observed that with the 1% formulations, the amount of lidocaine that could be extracted from the epidermis, dermis and receptor medium was significantly lower when compared to the 2% formulations. An application of 1 hour or longer was found to be necessary to observe any significant amount of lidocaine delivery.

TABLE 30
Influence of application time on lidocaine permeation profiles
as released from hydrogel-containing medical articles according
to the invention that had been loaded with either a 2% or 1%
caffeine solution by weight. Each value represents the average
cumulative amount of lidocaine in μg (and % applied dose)
recovered in the different compartments at the end of a 15-minute
period as obtained from eight samples.

| | | 2% lidocaine | | 2% lidocaine | | 2% lidocaine | |
|---|---|---|---|---|---|---|---|
| | | pH 3.0 | | pH 5.5 | | pH 7.0 | |
| 15 MINUTES | | Avg. | ±Sd | Avg. | ±Sd | Avg. | ±Sd |
| Receptor | μg | 0.00 | 0.00 | 0.00 | 0.00 | 1.99 | 5.27 |
| Fluid | % | 0.00 | 0.00 | 0.00 | 0.00 | 0.00 | 0.00 |
| Epidermis | μg | 0.00 | 0.00 | 0.00 | 0.00 | 1.52 | 2.36 |
| | % | 0.00 | 0.00 | 0.00 | 0.00 | 0.05 | 0.08 |
| Dermis | μg | 0.00 | 0.00 | 1.63 | 4.61 | 1.72 | 1.20 |
| | % | 0.00 | 0.00 | 0.07 | 0.20 | 0.06 | 0.04 |
| Hydrogel | μg | 2361.41 | 136.78 | 2174.86 | 52.00 | 2488.89 | 270.84 |
| | % | 104.64 | 6.06 | 93.08 | 2.23 | 89.34 | 9.72 |
| Washings | μg | 60.31 | 16.33 | 56.95 | 28.64 | 69.89 | 26.50 |
| | % | 2.67 | 0.72 | 2.44 | 1.23 | 2.51 | 0.95 |
| Mass | μg | 2421.71 | 130.82 | 2233.44 | 59.34 | 2564.01 | 254.59 |
| Balance | % | 107.32 | 5.80 | 95.59 | 2.54 | 92.04 | 9.14 |

| | | 1% lidocaine | | 1% lidocaine | | 1% lidocaine | |
|---|---|---|---|---|---|---|---|
| | | pH 3.0 | | pH 5.5 | | pH 7.0 | |
| 15 MINUTES | | Avg. | ±Sd | Avg. | ±Sd | Avg. | Sd |
| Receptor | μg | 0.00 | 0.00 | 0.00 | 0.00 | 0.00 | 0.00 |
| Fluid | % | 0.00 | 0.00 | 0.00 | 0.00 | 0.00 | 0.00 |
| Epidermis | μg | 0.00 | 0.00 | 0.56 | 1.47 | 0.69 | 1.28 |
| | % | 0.00 | 0.00 | 0.05 | 0.12 | 0.05 | 0.09 |
| Dermis | μg | 0.00 | 0.00 | 1.20 | 3.17 | 1.60 | 4.52 |
| | % | 0.00 | 0.00 | 0.10 | 0.26 | 0.11 | 0.32 |
| Hydrogel | μg | 1035.24 | 166.86 | 949.75 | 144.72 | 1195.83 | 113.97 |
| | % | 83.16 | 13.40 | 78.87 | 12.02 | 85.12 | 8.11 |
| Washings | μg | 44.88 | 24.60 | 54.72 | 52.10 | 18.62 | 12.55 |
| | % | 3.61 | 1.98 | 4.54 | 4.33 | 1.33 | 0.89 |
| Mass | μg | 1080.12 | 172.56 | 1006.23 | 108.95 | 1216.73 | 112.98 |
| Balance | % | 86.77 | 13.86 | 83.56 | 9.05 | 86.61 | 8.04 |

TABLE 31
Influence of application time on lidocaine permeation profiles
as released from hydrogel-containing medical articles according
to the invention that had been loaded with either a 2% or 1%
caffeine solution by weight. Each value represents the average
cumulative amount of lidocaine in μg (and % applied dose)
recovered in the different compartments at the end of a 30-minute
period as obtained from eight samples.

| | | 2% lidocaine | | 2% lidocaine | | 2% lidocaine | |
|---|---|---|---|---|---|---|---|
| | | pH 3.0 | | pH 5.5 | | pH 7.0 | |
| 30 MINUTES | | Avg. | ±Sd | Avg. | ±Sd | Avg. | ±Sd |
| Receptor | μg | 0.00 | 0.00 | 0.00 | 0.00 | 22.82 | 28.00 |
| Fluid | % | 0.00 | 0.00 | 0.00 | 0.00 | 0.82 | 1.00 |
| Epidermis | μg | 0.00 | 0.00 | 0.00 | 0.00 | 6.20 | 3.04 |
| | % | 0.00 | 0.00 | 0.00 | 0.00 | 0.22 | 0.11 |
| Dermis | μg | 1.00 | 2.82 | 3.32 | 2.82 | 17.06 | 10.81 |
| | % | 0.04 | 0.13 | 0.14 | 0.12 | 0.61 | 0.39 |
| Hydrogel | μg | 2410.58 | 161.32 | 2153.49 | 88.16 | 2287.35 | 328.48 |
| | % | 106.82 | 7.15 | 92.17 | 3.77 | 82.11 | 11.79 |
| Washings | μg | 80.82 | 61.99 | 68.13 | 19.23 | 185.63 | 207.33 |
| | % | 3.58 | 2.75 | 2.92 | 0.82 | 6.66 | 7.44 |
| Mass | μg | 2492.40 | 169.65 | 2224.93 | 100.30 | 2518.27 | 200.24 |
| Balance | % | 110.45 | 7.52 | 95.22 | 4.29 | 90.40 | 7.19 |
| | | 1% lidocaine | | 1% lidocaine | | 1% lidocaine | |
| | | pH 3.0 | | pH 5.5 | | pH 7.0 | |
| 30 MINUTES | | Avg. | ±Sd | Avg. | ±Sd | Avg. | ±Sd |
| Receptor | μg | 0.00 | 0.00 | 4.27 | 7.96 | 1.18 | 3.34 |
| Fluid | % | 0.00 | 0.00 | 0.35 | 0.66 | 0.08 | 0.24 |
| Epidermis | μg | 0.00 | 0.00 | 0.95 | 2.70 | 2.35 | 1.79 |
| | % | 0.00 | 0.00 | 0.08 | 0.22 | 0.17 | 0.13 |
| Dermis | μg | 0.00 | 0.00 | 3.93 | 7.95 | 3.14 | 3.36 |
| | % | 0.00 | 0.00 | 0.33 | 0.66 | 0.22 | 0.24 |
| Hydrogel | μg | 986.89 | 112.75 | 981.75 | 186.46 | 1228.03 | 107.07 |
| | % | 79.28 | 9.06 | 81.52 | 15.48 | 87.41 | 7.62 |
| Washings | μg | 52.94 | 41.23 | 58.56 | 32.69 | 27.78 | 13.64 |
| | % | 4.25 | 3.31 | 4.86 | 2.71 | 1.98 | 0.97 |
| Mass | μg | 1039.82 | 104.21 | 1049.47 | 155.21 | 1262.48 | 110.75 |
| Balance | % | 83.53 | 8.37 | 87.15 | 12.89 | 89.86 | 7.88 |

TABLE 32
Influence of application time on lidocaine permeation profiles
as released from hydrogel-containing medical articles according
to the invention that had been loaded with either a 1% or 2%
caffeine solution by weight. Each value represents the average
cumulative amount of lidocaine in μg (and % applied dose)
recovered in the different compartments at the end of a 1-hour
period as obtained from eight samples.

| | | 2% lidocaine | | 2% lidocaine | | 2% lidocaine | |
|---|---|---|---|---|---|---|---|
| | | pH 3.0 | | pH 5.5 | | pH 7.0 | |
| ONE HOUR | | Avg. | ±Sd | Avg. | ±Sd | Avg. | ±Sd |
| Receptor | μg | 0.00 | 0.00 | 0.00 | 0.00 | 10.11 | 7.36 |
| Fluid | % | 0.00 | 0.00 | 0.00 | 0.00 | 0.36 | 0.26 |
| Epidermis | μg | 0.00 | 0.00 | 0.00 | 0.00 | 5.78 | 3.13 |
| | % | 0.00 | 0.00 | 0.00 | 0.00 | 0.21 | 0.11 |
| Dermis | μg | 0.00 | 0.00 | 3.67 | 2.56 | 12.44 | 4.61 |
| | % | 0.00 | 0.00 | 0.16 | 0.11 | 0.45 | 0.17 |
| Hydrogel | μg | 2099.71 | 166.23 | 2202.34 | 121.79 | 2306.20 | 237.53 |
| | % | 93.05 | 7.37 | 94.26 | 5.21 | 82.78 | 8.53 |
| Washings | μg | 89.79 | 80.77 | 98.84 | 16.04 | 96.72 | 38.28 |
| | % | 3.98 | 3.58 | 4.23 | 0.69 | 3.47 | 1.37 |
| Mass | μg | 2189.50 | 189.31 | 2304.85 | 124.44 | 2431.25 | 245.53 |
| Balance | % | 97.03 | 8.39 | 98.65 | 5.33 | 87.27 | 8.81 |
| | | 1% lidocaine | | 1% lidocaine | | 1% lidocaine | |
| | | pH 3.0 | | pH 5.5 | | pH 7.0 | |
| ONE HOUR | | Avg. | ±Sd | Avg. | ±Sd | Avg. | ±Sd |
| Receptor | μg | 2.91 | 8.23 | 0.00 | 0.00 | 4.98 | 5.15 |
| Fluid | % | 0.23 | 0.66 | 0.00 | 0.00 | 0.35 | 0.37 |
| Epidermis | μg | 0.00 | 0.00 | 1.50 | 2.23 | 2.12 | 2.41 |
| | % | 0.00 | 0.00 | 0.12 | 0.19 | 0.15 | 0.17 |
| Dermis | μg | 0.65 | 1.85 | 2.46 | 4.22 | 6.01 | 4.03 |
| | % | 0.05 | 0.15 | 0.20 | 0.35 | 0.43 | 0.29 |
| Hydrogel | μg | 837.12 | 152.68 | 1025.83 | 119.06 | 1188.13 | 121.23 |
| | % | 67.25 | 12.26 | 85.18 | 9.89 | 84.57 | 8.63 |
| Washings | μg | 71.69 | 61.67 | 65.50 | 40.32 | 56.47 | 40.30 |
| | % | 5.76 | 4.95 | 5.44 | 3.35 | 4.02 | 2.87 |
| Mass | μg | 912.37 | 167.08 | 1095.29 | 121.22 | 1257.69 | 101.73 |
| Balance | % | 73.29 | 13.42 | 90.95 | 10.07 | 89.52 | 7.24 |

TABLE 33
Influence of application time on lidocaine permeation profiles
as released from hydrogel-containing medical articles according
to the invention that had been loaded with either a 1% or 2%
caffeine solution by weight. Each value represents the average
cumulative amount of lidocaine in μg (and % applied dose)
recovered in the different compartments at the end of a 2-hour
period as obtained from eight samples.

| | | 2% lidocaine | | 2% lidocaine | | 2% lidocaine | |
|---|---|---|---|---|---|---|---|
| | | pH 3.0 | | pH 5.5 | | pH 7.0 | |
| TWO HOURS | | Avg. | ±Sd | Avg. | ±Sd | Avg. | ±Sd |
| Receptor | μg | 2.02 | 3.78 | 0.00 | 0.00 | 23.15 | 15.20 |
| Fluid | % | 0.09 | 0.17 | 0.00 | 0.00 | 0.83 | 0.55 |
| Epidermis | μg | 0.00 | 0.00 | 4.37 | 6.66 | 8.98 | 4.52 |
| | % | 0.00 | 0.00 | 0.19 | 0.29 | 0.32 | 0.16 |
| Dermis | μg | 0.00 | 0.00 | 14.20 | 37.52 | 12.19 | 7.25 |
| | % | 0.00 | 0.00 | 0.61 | 1.61 | 0.44 | 0.26 |
| Hydrogel | μg | 2124.55 | 245.04 | 2137.68 | 205.46 | 2131.29 | 240.81 |
| | % | 94.15 | 10.86 | 91.49 | 8.79 | 76.50 | 8.64 |
| Washings | μg | 172.26 | 35.68 | 140.80 | 95.03 | 197.32 | 125.84 |
| | % | 7.63 | 1.58 | 6.03 | 4.07 | 7.08 | 4.52 |
| Mass | μg | 2298.83 | 246.53 | 2297.04 | 144.92 | 2372.93 | 216.67 |
| Balance | % | 101.87 | 10.92 | 98.31 | 6.20 | 85.18 | 7.78 |

| | | 1% lidocaine | | 1% lidocaine | | 1% lidocaine | |
|---|---|---|---|---|---|---|---|
| | | pH 3.0 | | pH 5.5 | | pH 7.0 | |
| TWO HOURS | | Avg. | ±Sd | Avg. | ±Sd | Avg. | Sd |
| Receptor | μg | 0.00 | 0.00 | 0.00 | 0.00 | 7.06 | 8.51 |
| Fluid | % | 0.00 | 0.00 | 0.00 | 0.00 | 0.50 | 0.61 |
| Epidermis | μg | 0.00 | 0.00 | 2.23 | 1.62 | 3.84 | 2.44 |
| | % | 0.00 | 0.00 | 0.19 | 0.13 | 0.27 | 0.17 |
| Dermis | μg | 1.02 | 1.89 | 0.89 | 2.53 | 5.73 | 2.90 |
| | % | 0.08 | 0.15 | 0.07 | 0.21 | 0.41 | 0.21 |
| Hydrogel | μg | 933.58 | 94.18 | 1069.88 | 73.77 | 1213.46 | 136.06 |
| | % | 74.99 | 7.57 | 88.84 | 6.13 | 86.37 | 9.68 |
| Washings | μg | 146.40 | 107.61 | 75.04 | 24.15 | 45.20 | 14.85 |
| | % | 11.76 | 8.64 | 6.23 | 2.01 | 3.22 | 1.06 |
| Mass | μg | 1081.00 | 117.37 | 1148.05 | 91.49 | 1275.28 | 118.95 |
| Balance | % | 86.84 | 9.43 | 95.33 | 7.60 | 90.77 | 8.47 |

Data obtained from this experiment suggest that the medical articles of the invention are good candidates for short-term release of lidocaine. The data also suggest that the absorption profile of lidocaine is dependent on the drug loading of the medical articles, the pH of the hydrogel included in the medical article, and the amount of time that the medical article is applied on the skin.

3. Conclusion

The percutaneous absorption studies demonstrate that the hydrogel-containing medical articles of the invention can effectively deliver hydrophilic active ingredients across intact skin. Depending on the physico-chemical properties of the active ingredients, the release of the drug may be modulatedd at least by the drug loading, pH, and protein composition of the hydrogels, as well as the application time. Moreover, this release may be percutaneous or exclusively cutaneous. As a result, the formulation of the hydrogel-containing medical articles of the invention may be designed by taking into account the balance between the desirable biological effects and the toxicity of the drug (if any).

### EXAMPLE 18

### Wound Healing Effects of Hydrogel-Containing Medical Articles

This series of studies evaluated the wound healing effects of wound dressings including the hydrogel of Example 7 in vivo. Specifically, the tested wound dressings contain hydrogels prepared by crosslinking PEG 8 kDa with hydrolyzed soy protein as described in Example 7 that were then loaded with an aqueous solution having a pH of 5.5 and containing NaCl (0.9 wt. %), LIQUID GERMALL® PLUS (0.5 wt. %), EDTA (0.2 wt. %), and NaH₂PO₄.2H₂O (0.16 wt. %). Such wound dressings will be referred to as “PEG-soy hydrogel wound dressings” throughout this example.

A. Wound Healing Effects on Rats

Full Thickness Wounds

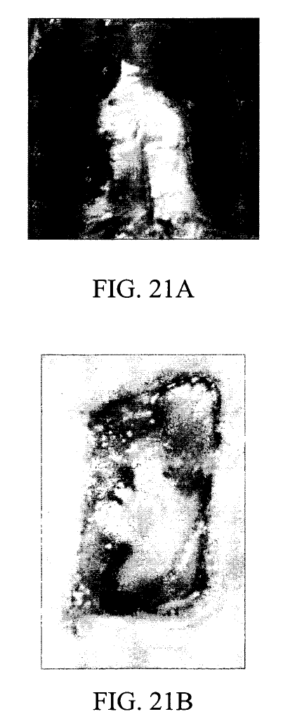

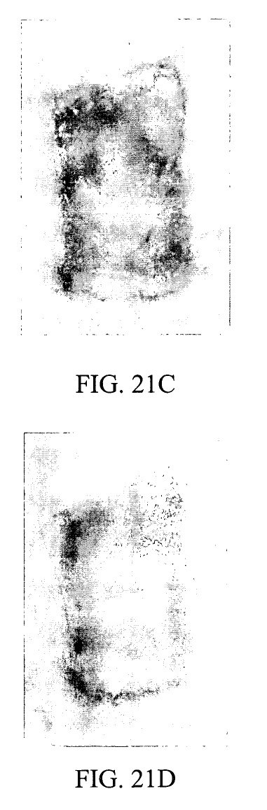

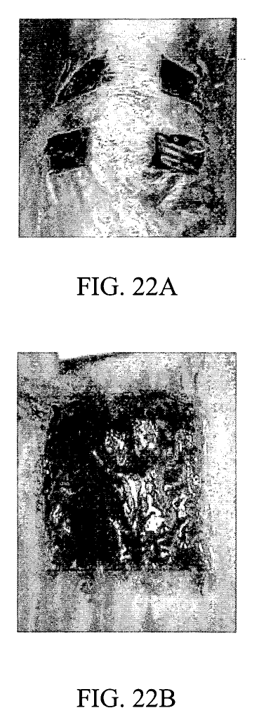

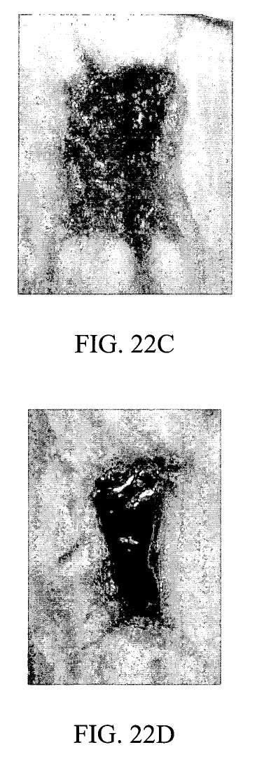

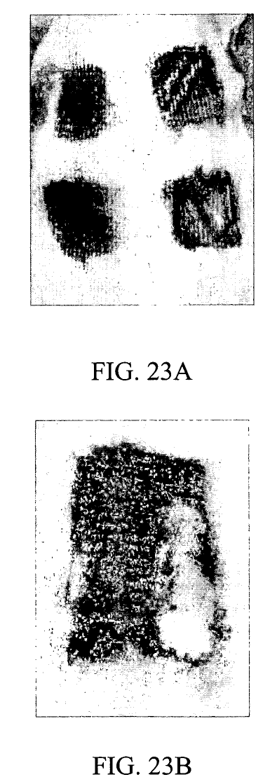

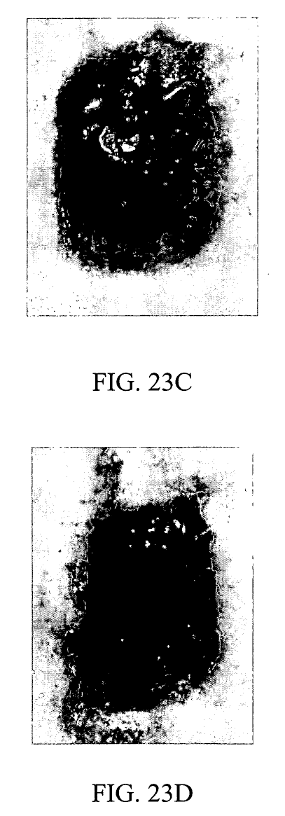

Rats were subjected to full thickness wounds on their back, the wounds having a size of 1.5 cm×1.5 cm. The following wound dressings were applied topically to the region of the wound: i) an ADAPTIC® non-adhering dressing (marketed by Johnson & Johnson), ii) an TEGADERM™ semi-permeable adhesive dressing (as described above, and marketed by 3M), or iii) a PEG-soy hydrogel wound dressing. Animals were then bandaged identically, and the dressings were changed three times over a 6-day period. From Day 6 to Day 12, all the wounds were kept at ambient air conditions. FIGS. 21A to 21D, 22A to 22D, and 23A to 23D are photographic representations of the wounds before treatment (FIGS. 21A, 22A, and 23A) and after 2 days (FIGS. 21B, 22B, and 23B), 4 days (FIGS. 21C, 22C, and 23C) and 6 days (FIGS. 21D, 22D, and 23D) of treatment with the PEG-soy hydrogel wound dressing, TEGADERM™ semi-permeable adhesive dressing, and ADAPTIC® non-adhering dressing, respectively.

Results

As shown in FIGS. 21A to 21D, 22A to 22D, and 23A to 23D, wounds stopped bleeding after the first 48 hours when they were treated with the PEG-soy hydrogel wound dressing, whereas bleeding was observed at every bandage renewal for both the TEGADERM™ semi-permeable adhesive dressing and the ADAPTIC® non-adhering dressing. Most of this bleeding was due to destruction of the weak, newly synthetized granulation tissue by the comparison bandages themselves. It also was observed that the PEG-soy hydrogel wound dressing placed onto the wound surface prevented contraction of the wound that took place from the fourth day for the wounds treated with the TEGADERM™ semi-permeable adhesive dressing. As a consequence, the PEG-soy hydrogel wound dressing provided a greater healed surface.

Despite this observation, wounds treated with the PEG-soy hydrogel wound dressing, as soon as Day 2, were colonized by a thick granulation tissue. Reepithelialization was complete after 6 days of treatment with the PEG-soy hydrogel wound dressing. Wounds treated with the PEG-soy hydrogel wound dressing were highly vascularized until Day 12. On the other hand, wounds treated with TEGADERM™ semi-permeable adhesive dressing presented granulation tissue at Day 4 and were not closed at Day 6. Although some granulation tissue was observed at Day 2, wounds treated with ADAPTIC® non-adhering dressing presented a slight contraction and were not closed at Day 12. Also, as wounds were kept in the air environment, the formation of a slight crust, which disappeared on Day 12, was observed for wounds treated with the PEG-soy hydrogel wound dressing.

From the data obtained, it can be concluded that the PEG-soy hydrogel wound dressing enhances wound healing in rats by (i) preventing infection of the wound, (ii) providing a moist environment that facilitates cell growth, and (iii) offering an adhesive but non-sticky wound care that can be easily removed from the wound without destroying the neo-synthesized tissues.

B. Wound Healing Effects on Pigs

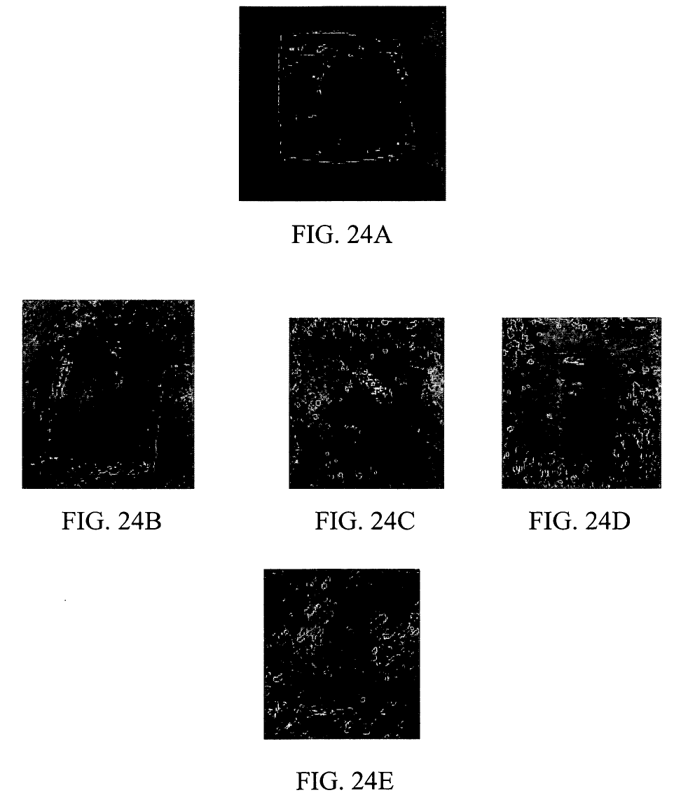

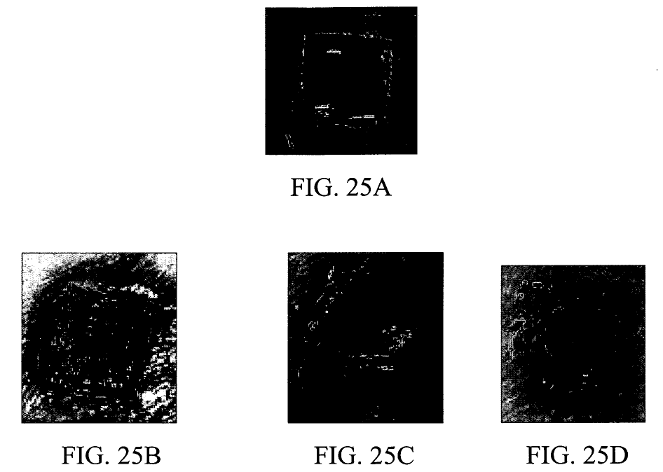

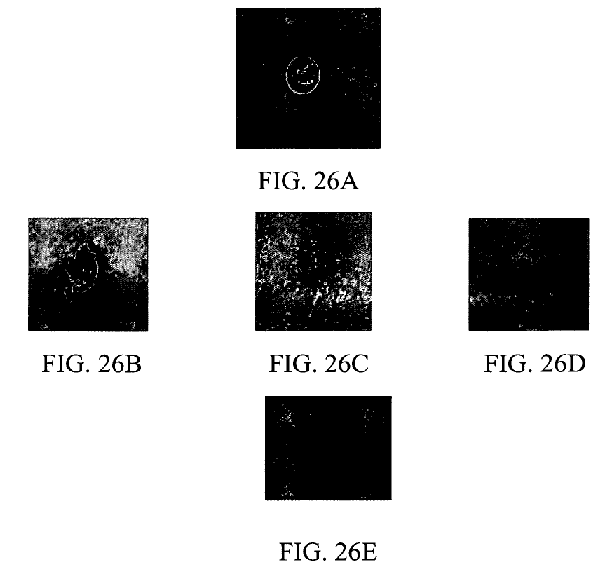

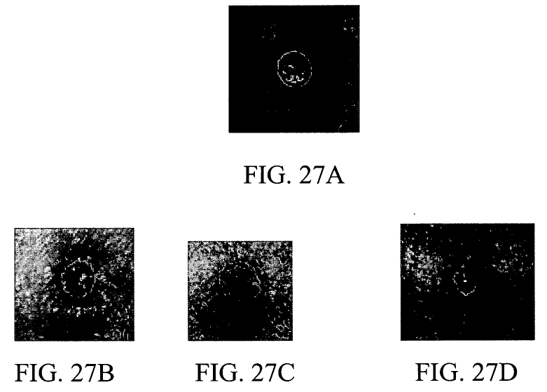

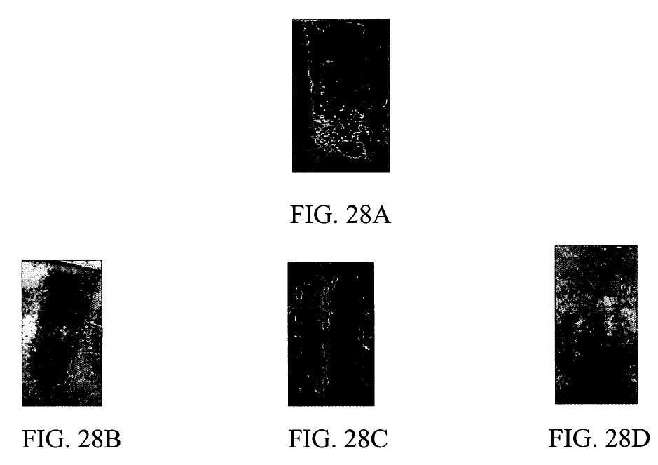

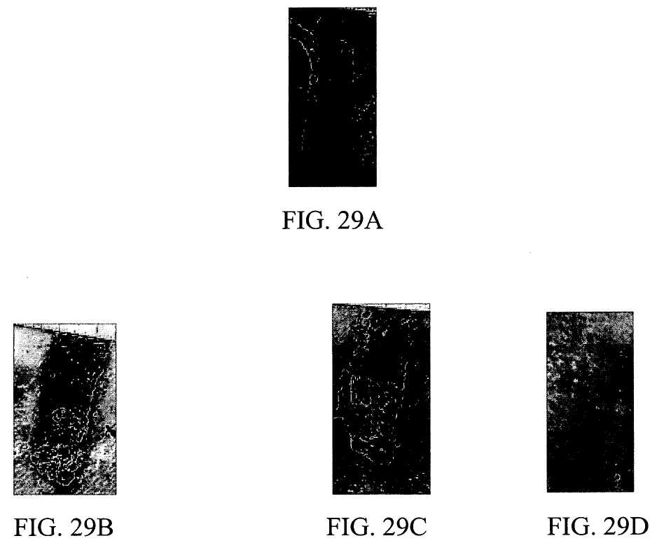

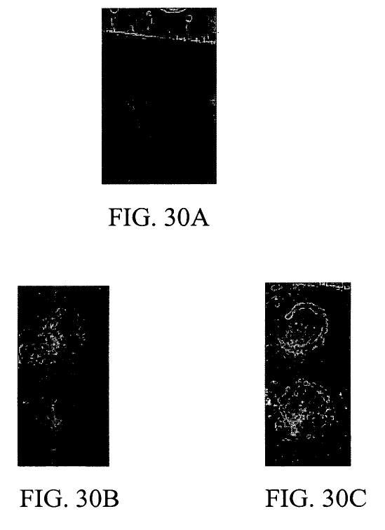

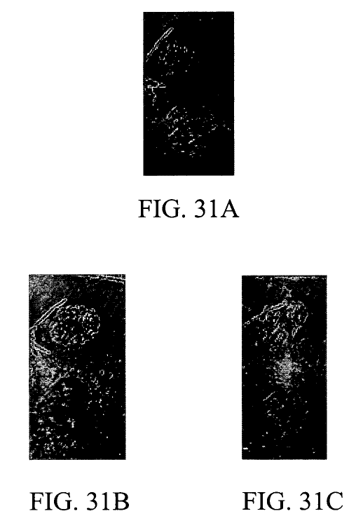

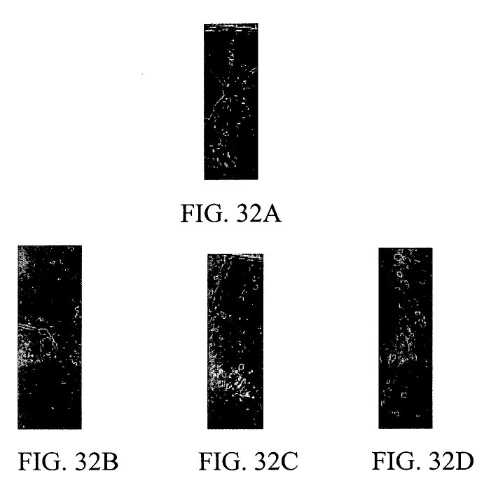

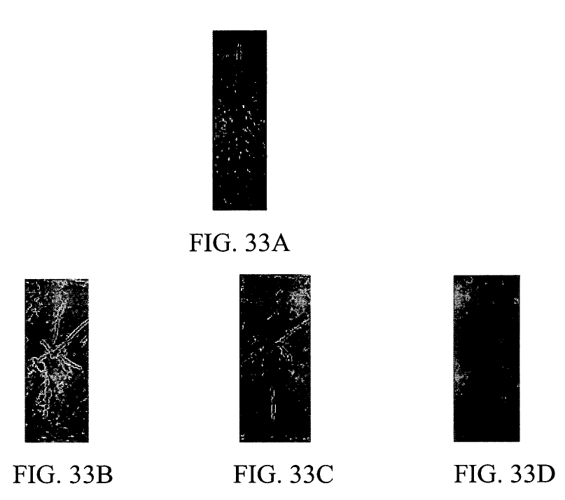

Four pigs were studied to assess the efficacy of hydrogel-containing medical articles of the invention in healing different types of wounds. On the back of each pig, the following wounds were created: i) a full thickness wound having a size of 2 cm×2 cm, ii) a full thickness wound having a size of 1 cm diameter, iii) a partial thickness wound having a thickness of 300 μm and a size of 3 cm×1 cm, iv) a 1 cm diameter chemical burn, v) a 1 cm diameter thermal burn, and vi) a 3 cm surgical incision. FIGS. 24A and 25A show the initial appearance of an exemplary 2 cm×2 cm full thickness wound on a pig, and FIGS. 26A and 27A show the initial appearance of an exemplary 1 cm diameter full thickness wound on a pig. FIGS. 28A and 29A show the initial appearance of an exemplary 1 cm×3 cm partial thickness wound on a pig. FIGS. 30A and 31A show the initial appearance of an exemplary 1 cm diameter chemical burn and an exemplary 1 cm diameter thermal burn on a pig. FIGS. 32A and 33A show the initial appearance of an exemplary surgical incision on a pig. The following wound dressings were applied topically to the region of the wound: i) a TEGADERM™ semi-permeable adhesive dressing (as described above, marketed by 3M) or ii) a PEG-soy hydrogel wound dressing. Whenever a PEG-soy hydrogel wound dressing was applied in this experiment, a secondary dressing (the TEGADERM™ adhesive dressing described above) was used to cover the PEG-soy hydrogel wound dressing to prevent water depletion. Animals were then bandaged identically, and the dressings were changed three times every week over a 21-day period.

1. Full Thickness Wounds

FIGS. 24B-24E are photographic representations of the 2 cm×2 cm wounds after 4, 7, 10 and 21 days of treatment with the PEG-soy hydrogel wound dressing, respectively. FIGS. 25B-25D are photographic representations of the 2 cm×2 cm wounds after 4, 7, and 10 days of treatment with the TEGADERM™ semi-permeable adhesive dressing, respectively. FIGS. 26B-26E are photographic representations of the 1 cm diameter wounds after 4, 7, 10 and 21 days of treatment with the PEG-soy hydrogel wound dressing, respectively. FIGS. 27B-27D are photographic representations of the 1 cm diameter wounds after 4, 7 and 10 days of treatment with the TEGADERM™ semi-permeable adhesive dressing, respectively.

Results

As shown in FIGS. 24B-24E and in Table 34, at Day 4, granulation tissue that covered the surface of the wound was observed on the 2 cm×2 cm full thickness wounds treated with the PEG-soy hydrogel wound dressing. The PEG-soy hydrogel wound dressing appeared clean with no signs of infection. Moreover, an absence of inflammatory signs was also observed. Neither erythema nor edema were found after 4 days of treatment with the PEG-soy hydrogel wound dressing. Additionally, it was observed that the neo-synthesized epidermis had colonized almost 50% of the surface wound as early as Day 4. Complete wound closure without visible scar was observed after 21 days of treatment with the PEG-soy hydrogel wound dressing. Normal hair also had started growing around and covering part of the wound site.

On the other hand and as shown in FIGS. 25B-25D and in Table 34, the 2 cm×2 cm full thickness wound treated with the TEGADERM™ semi-permeable adhesive dressing presented a high amount of wound fluid, leaving the wound partially infected (as indicated by its appearance and a foul odor) after 4 days of treatment. Moreover, less granulation tissue and high inflammatory signs, such as erythema and edema, were found after 4 days of treatment. Minimal epidermis (24%) had colonized the wound, leaving it fairly open at Day 4. Epithelialization almost took place at Day 7. Unfortunately, observation of the wound after Day 12 was impossible due to the death of the animals that were treated with the TEGADERM™ wound dressing.

As shown in FIGS. 26B-26E and FIGS. 27B-27D and in Table 34, when the full thickness wound size is 1 cm in diameter, similar results to those described for FIGS. 24B-24E and FIGS. 25B-25D are observed except that the inflammatory phase appeared to be less important for the wounds treated with the TEGADERM™ semi-permeable adhesive dressing.

It can be concluded from this study that the PEG-soy hydrogel wound dressing promotes wound healing by (i) reducing both the intensity and the duration of the inflammatory phase, (ii) promoting epithelialization via its moist environment, and (iii) preventing the formation of a scar.

TABLE 34
Percentage of wound closure as a function of time. Each value presented below is
an average number collected from 4 wounds and is associated with its standard deviation.
“Hydrogel” refers to the PEG-soy hydrogel wound dressing.

| | | | |
|---|---|---|---|
| DAY 4 | DAY 7 | DAY 10 | DAY 21 |

Full thickness wound (2 cm × 2 cm)

| | | | | |
|---|---|---|---|---|
| Hydrogel | 51.55 ± 7.61 | 52.06 ± 7.53 | 74.13 ± 1.59 | 96.18 ± 1.25 |
| TEGADERM ™ | 24.21 ± 3.46 | 51.55 ± 7.61 | 78.69 ± 3.35 | nd |

Full thickness wound (1 cm diameter)

| | | | | |
|---|---|---|---|---|
| Hydrogel | 65.55 ± 0.00 | 84.85 ± 0.00 | 85.60 ± 0.00 | 99.22 ± 0.00 |
| TEGADERM ™ | 29.10 ± 11.76 | 52.15 ± 18.95 | 82.36 ± 8.54 | nd |

Partial thickness wound (1 cm × 3 cm)

| | | | | |
|---|---|---|---|---|
| Hydrogel | 56.94 ± 0.00 | 100.00 ± 0.00 | 100.00 ± 0.00 | 100 ± 0.00 |
| TEGADERM ™ | 2.20 ± 0.00 | 70.65 ± 3.61 | 100 ± 0.00 | nd |

2. Partial Thickness Wounds

FIGS. 28B-28D and FIGS. 29B-29D are photographic representations of the 1 cm×3 cm partial thickness wound on a pig after 4 days (FIGS. 28B and 29B), 7 days (FIGS. 28C and 29C) and 12 days (FIGS. 28D and 29D) of treatment with the PEG-soy hydrogel wound dressing and the TEGADERM™ semi-permeable adhesive dressing, respectively.

Results

As shown in FIGS. 28B-28D and Table 34, after 4 days of treatment, the wound treated by the PEG-soy hydrogel wound dressing presented no signs of inflammation (no edema or erythema) or infection and was more than 50% colonized by a neo-synthesized epidermis. The wound was clean with no sign of infection. Wound closure was completed by Day 7 without scar tissue, and the color of the wound site was very similar to the surrounding normal tissue.

However, as shown in FIGS. 29B-29D, after 4 days of treatment, the wound treated by the TEGADERM™ dressing presented large amounts of wound fluid, leaving the wound quite dirty with visible edema and erythema. After 7 days of treatment with the TEGADERM™ dressing, the wound was mainly scar tissue with a color considerably different from the surrounding normal tissue. Complete closure of the wound took place after 10 days of treatment with the TEGADERM™ dressing.

It can be concluded from this study that the PEG-soy hydrogel wound dressing promotes wound healing of partial thickness wounds by (i) reducing both the intensity and the duration of the inflammatory phase, (ii) enhancing epithelialization rate, (iii) accelerating wound closure, and (iv) preventing the formation of a scar.

3. Other Wounds

FIGS. 30B and 30C and FIGS. 31B and 31C are photographic representations of the thermal and chemical burns on the pigs after 4 days (FIGS. 30B and 31B) and 7 days (FIGS. 30C and 31C) of treatment with the PEG-soy hydrogel wound dressing and the TEGADERM™ semi-permeable adhesive dressing, respectively. FIGS. 32B-32D and FIGS. 33B-33D are photographic representations of the surgical incision on the pigs after 4 days (FIGS. 32B and 33B), 7 days (FIGS. 32C and 33C), and 10 days (FIGS. 32D and 33D) of treatment with the PEG-soy hydrogel wound dressing and the TEGADERM™ semi-permeable adhesive dressing, respectively.

Results

As shown in FIGS. 30B and 30C, 31B and 31C, 32B to 32D, and 33B to 33D, regardless of the wound type and the treatment, all the wounds were healed after 4 days of treatment with both the PEG-soy hydrogel wound dressing and the TEGADERM™ semi-permeable adhesive dressing.

Together, these three studies demonstrated that the PEG-soy hydrogel wound dressings were very effective in promoting wound healing compared to the commercially available wound dressings tested, both in terms of the rate of healing and the improvement in wound appearance.

C. Wound Healing in Humans

1. Acute Wounds

a. Lacerations and Traumatic Wounds

In one case, a woman received an injury from a door that fell on her right wrist. The trauma caused several deep lacerations (FIG. 34A). A PEG-soy hydrogel wound dressing was applied immediately after injury and renewed every day. A TEGADERM™ secondary dressing (a transparent and self-adhesive film as described above) was used to cover the PEG-soy hydrogel wound dressing. FIGS. 34B and 34C are photographic representations of the lacerations after 24 hours (FIG. 34B) and 48 hours (FIG. 34C) of treatment with the PEG-soy hydrogel wound dressing, respectively.

As shown in FIGS. 34B and 34C, after 24 hours of treatment with the PEG-soy hydrogel wound dressing, the inflammation signs disappeared and the wound started to heal. Complete re-epithelialization was obtained in 48 hours without local complications, such as infections, and with a sensation of comfort and freshness. An application of the PEG-soy hydrogel wound dressing eliminated the initial signs of inflammation (pain, itching, heat, and redness).

It can be concluded that the PEG-soy hydrogel wound dressing provided a beneficial healing environment. In fact, acceleration of wound healing and improvement of scarring from deep wounds are important clinical goals in emergency medicine.

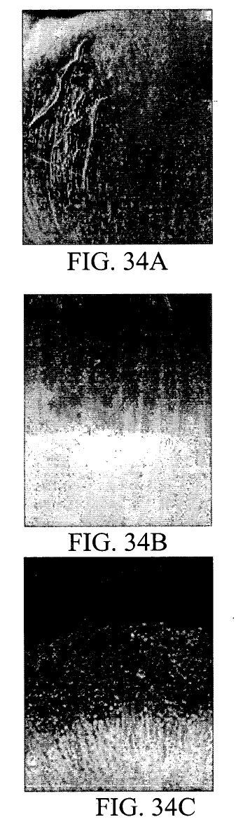

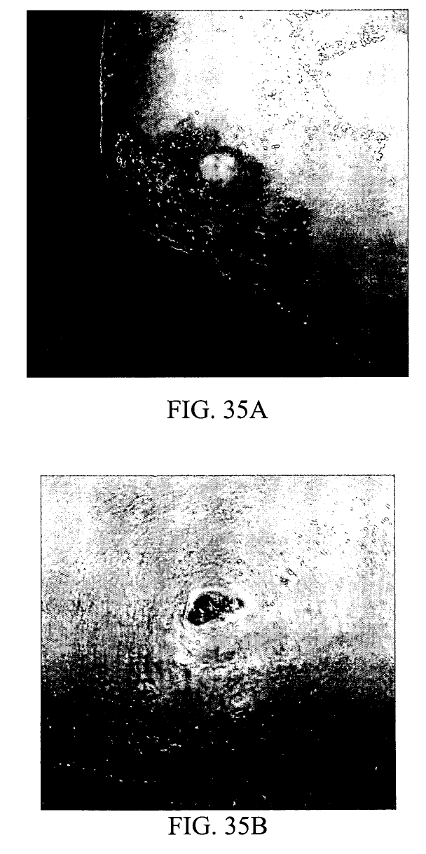

In a second case, a 10 year-old boy was injured by striking a wall, leading to several deep lacerations and severe bleeding on his right arm (FIG. 35A). The patient had to wait 5 hours before being treated in hospital. A PEG-soy hydrogel wound dressing was applied after cleaning the wound and renewed every day. A TEGADERM™ secondary dressing (a transparent and self-adhesive film as described above) was used to cover the PEG-soy hydrogel wound dressing. FIG. 35B is a photographic representation of the lacerations after 72 hours of treatment with the PEG-soy hydrogel wound dressing.

It was observed that after 24 hours of treatment with the PEG-soy hydrogel wound dressing the inflammation signs disappeared and the wound started to heal. As shown in FIG. 35B, complete re-epithelialization was obtained in 72 hours without local complications, such as infections, and with a sensation of comfort and freshness. Additionally, application of the PEG-soy hydrogel wound dressing calmed the initial signs of inflammation (pain, itching, heat, and redness).

It can be concluded that the PEG-soy hydrogel wound dressing provided a beneficial healing environment. Retention of biologic fluids over the wound prevents desiccation of denuded dermis or deeper tissues and allowed faster and unimpeded migration of keratinocytes onto the wound surface.

b. Burns

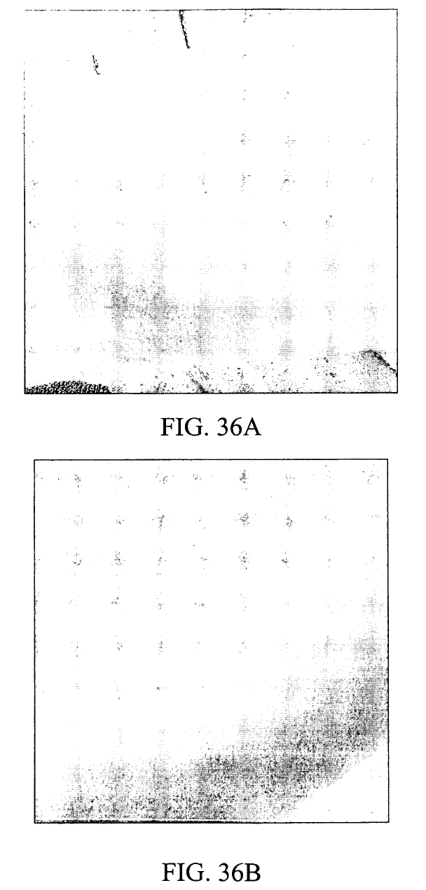

A 23 year-old woman had a first degree burn on her left arm caused by boiling water. The woman displayed signs of the early stages of blister formation, felt a lot of pain, displayed edema, and felt a sensation of discomfort (FIG. 36A). A PEG-soy hydrogel wound dressing was applied immediately after injury and renewed every day. A TEGADERM™ secondary dressing (a transparent and self-adhesive film as described above) was used to cover the PEG-soy hydrogel wound dressing. FIG. 36B is a photographic representation of the burn after 48 hours of treatment with the PEG-soy hydrogel wound dressing.

After 24 hours of treatment with the PEG-soy hydrogel wound dressing, the inflammation reaction disappeared. Additionally, blister formation was ceased, and pain was relieved and replaced with a good sensation. As shown in FIG. 36B, after 48 hours of treatment, the inflammation signs completely disappeared and the burn started to heal. Complete re-epithelialization was obtained in 72 hours without local complications, such as infection, and with a great sensation of comfort and freshness.

It can be concluded that the PEG-soy hydrogel wound dressing relieved the initial signs of inflammation (pain, itching, heat, and redness) very well. The PEG-soy hydrogel wound dressing provided a beneficial healing environment which was moist and which allowed a faster and better epithelialization without leaving any scar.

c. Radiodermatitis

Ten irradiated patients were studied to demostrate the efficacy the PEG-soy hydrogel wound dressing in preventing and treating radio-dermatitis in neoadjuvant skin areas that were irradiated by doses greater than 45-50 Gray. The areas that are most susceptible to irradiation-mediated skin disorders, when irradiated with doses exceeding 50 Gray, are cervical, breast, inguinal, perianal, and perineum areas, and also any skin areas.

This study showed that no redness or sores appeared after 24 hours of treatment. The PEG-soy hydrogel wound dressing relieved the signs of inflammation immediately after the radiotherapy (pain, itching, heat, and redness). It can be concluded that the PEG-soy hydrogel wound dressing delayed appearance of dermatitis or showed dermatitis of only a minor degree.

2. Chronic Wounds

Ehlers-Danlos syndrome (EDS) is a heterogeneous group of heritable connective tissue disorders, characterized by articular point) hypermobility, skin extensibility, and tissue fragility.

a. Infected Wound

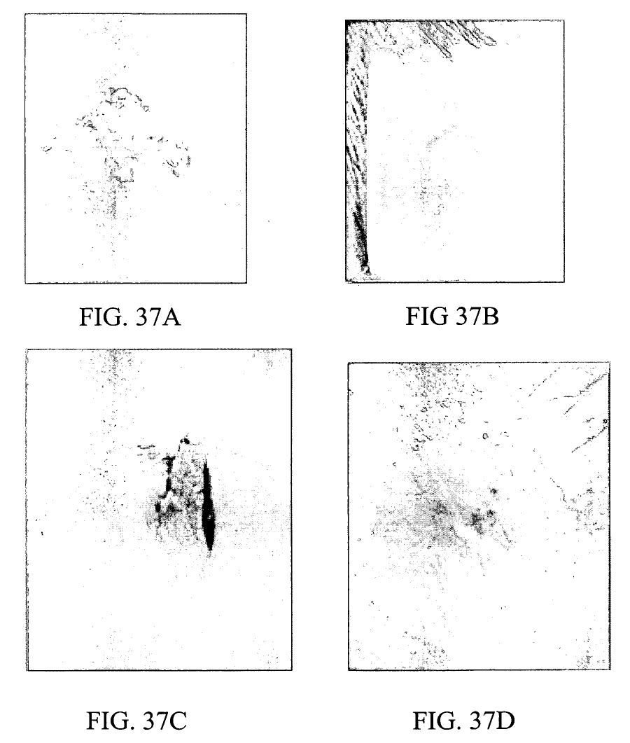

A 22 year-old woman, with type V Ehlers-Danlos Syndrome, who had an infected wound on her right forearm just over a recent scar area, was studied. The woman reported that her wounds typically took between 2 and 3 months to completely close. The injury was caused by trauma due to a nail. The wound was cleaned and covered with an ordinary dressing. Two days later, she requested the use of the PEG-soy hydrogel wound dressing because her wound had changed. The wound had infection signs such as pain, increasing local temperature and erythema, and a yellow purulent exudate, as shown in FIG. 37A. The PEG-soy hydrogel wound dressing was applied after cleaning the wound and was changed every two days. A TEGADERM™ secondary dressing (a transparent and self-adhesive film as described above) was used to cover the PEG-soy hydrogel wound dressing. The treatment lasted 13 days, until a total closure of the wound without any infections was obtained. FIGS. 37B and 37C show the appearance of the wound after 48 hours of treatment with the PEG-soy hydrogel wound dressing. FIG. 37B shows the wound being covered by the PEG-soy hydrogel wound dressing. FIG. 37C shows the wound by itself with the PEG-soy hydrogel wound dressing having been removed. FIG. 37D shows the appearance of the wound after 13 days of treatment with the PEG-soy hydrogel wound dressing.

After 48 hours of treatment with the PEG-soy hydrogel wound dressing, the signs of infection were eliminated (FIGS. 37B and 37C). The treatment was fast and efficient as was judged by complete re-epithelialization and wound closure in 13 days (FIG. 37D). It can be concluded that the PEG-soy hydrogel wound dressing was effective in removing the infection and provided a moist environment, which had a favorable effect on epithelialization and wound closure, as well as producing minimal scarring.

b. Acute Infected Wound

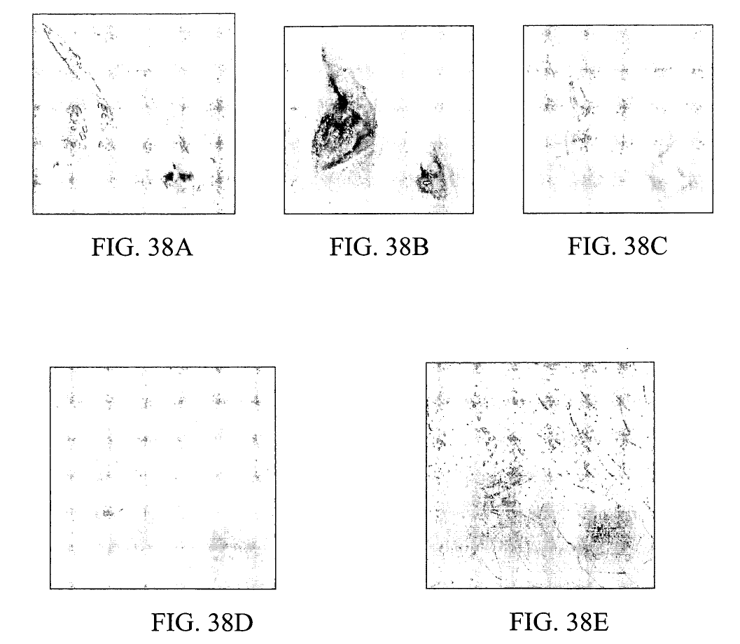

The same 22 year-old woman with Ehlers-Danlos Syndrome described above was hit by a dog over her left knee. She presented with three different wounds in form and size: (i) an irregular V-shaped wound measuring 2 cm on the long side and 1.5 cm on the short side; (ii) a second small wound of 0.5 cm in diameter close to the first wound; and (iii) another small wound of 0.4 cm in diameter on the left knee area (FIG. 38A). All the wounds were treated with the PEG-soy hydrogel wound dressing and covered by a TEGADERM™ secondary dressing as previously described. FIGS. 38B to 38E are photographic representations of the wounds after 10 days (FIG. 38B), 20 days (FIG. 38C), 28 days (FIG. 38D), and 38 days (FIG. 38E) of treatment with the PEG-soy hydrogel wound dressing, respectively.

As shown in FIGS. 38B-38E, after 24 hours of treatment with the PEG-soy hydrogel wound dressing, the signs of initial inflammation were decreased, and the wounds started to heal without any local infection episode (a frequent event where the wound healing is very slow and where there is a considerable gap). Complete re-epithelialization (wound closure) of the biggest wound was obtained in 38 days.

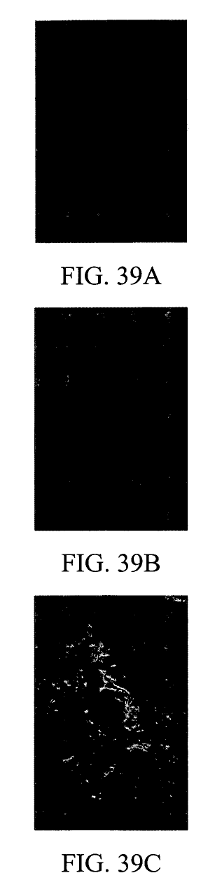

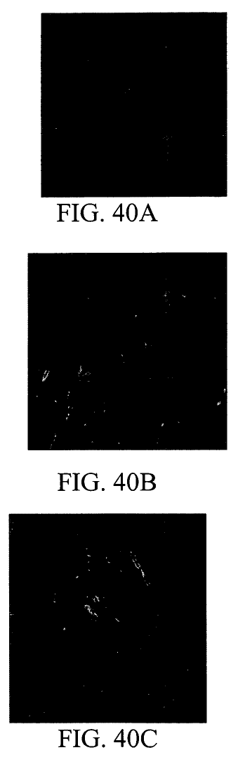

Eighteen days later, the same patient had another new wound due to a pressure shock accident. The wound was a flap of tissue in the shape of a V and measured 1.2 cm×1.2 cm (FIG. 39A). The wound was on her right heel, and it was closed by a medical professional with 4 mononylon points, but without closure of the wound border. She also had another small wound measuring 0.4 cm in diameter on the right knee area (FIG. 40A). The wounds were treated with the PEG-soy hydrogel wound dressing and covered by a TEGADERM™ secondary dressing as previously described. FIGS. 39B-39C and FIGS. 40B-40C are photographic representations of the wounds on her heel and her right knee and after 10 days (FIG. 39B and FIG. 40B) and 20 days (FIG. 39C and FIG. 40C) of treatment with the PEG-soy hydrogel wound dressing, respectively.

As shown in FIGS. 39A-39C and 40A-40C, after a 20-day treatment with the PEG-soy hydrogel wound dressing, all signs of initial inflammation were relieved (pain, itch, heat, and redness), and the wounds were closed without any local complication and with a sensation of comfort, freshness, and absence of pain as reported by the patient.

It can be concluded that the PEG-soy hydrogel wound dressing prevented infection of the wound and hypertrophic scar and promoted wound healing in patients having a genetic skin disorder. With conventional treatment of the chronic full thickness wounds (which are potentially infected), comparable results are normally obtained after a longer period of time.

Incorporation by Reference

The disclosures of each of the patent documents and scientific articles identified herein are expressly incorporated by reference herein.

### Other Embodiments

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

Other embodiments of the invention are within the following claims

1. A medical article comprising:
a hydrophilic water-swellable hydrogel comprising a crosslinked mixture of a biocompatible polymer and a protein, and
at least one of diazolidinyl urea and iodopropynyl butylcarbamate dispersed within the hydrophilic water-swellable hydrogel.

2. The medical article of claim 1, wherein the biocompatible polymer comprises polyethylene glycol.

3. The medical article of claim 1, wherein the protein comprises albumin.

4. The medical article of claim 3, wherein the albumin is obtained from a vegetal source.

5. The medical article of claim 4, wherein the vegetal source comprises a soybean.

6. The medical article of claim 1, wherein the medical article further comprises a support comprising a polymeric surface, wherein the hydrophilic water-swellable hydrogel is attached to the polymeric surface of the support.

7. The medical article of claim 1, wherein the medical article further comprises an in-dwelling member, the in-dwelling member comprising a first portion adapted to be inserted into the body of a patient and a second portion adapted to be exposed outside the body of a patient, wherein the hydrophilic water-swellable hydrogel is disposed about the in-dwelling member at a point along the second portion of the in-dwelling member.

8. A method for treating a wound, the method comprising
administering a first medical article to a wound, the first medical article comprising
a hydrophilic water-swellable hydrogel comprising a crosslinked mixture of a biocompatible polymer and a protein, and
at least one of diazolidinyl urea and iodopropynyl butylcarbamate dispersed within the hydrophilic water-swellable hydrogel;
such that wound healing occurs faster as compared to a wound being treated in an identical manner by a second medical article comprising a polyurethane membrane coated with a layer of an acrylic adhesive.

9. The method of claim 8, wherein the rate of wound healing is determined by measuring at least one criterion selected from a group consisting of reduction of wound size, amount of time to achieve wound closure, contrast between wound color and normal tissue color, signs of infection, and duration of the inflammatory phase.

10. A method for treating a wound, the method comprising
applying a medical article to an anatomical site of a patient, the medical article comprising
a hydrophilic water-swellable hydrogel comprising a crosslinked mixture of a biocompatible polymer and a protein; and
at least one of diazolidinyl urea and iodopropynyl butylcarbamate dispersed within the hydrophilic water-swellable hydrogel.

11. The method of claim 10, wherein the anatomical site comprises a topical site.

12. A method for treating a wound, the method comprising:
applying a medical article to an infected wound, the medical article comprising
a hydrating component comprising a hydrophilic water-swellable hydrogel comprising a crosslinked mixture of a biocompatible polymer and a protein, and
an oxidizing agent dispersed within said hydrogel, the oxidizing agent being in a therapeutically effective amount to generate an antimicrobial effect.

13. A method for preparing a medical article, the method comprising loading a hydrophilic water-swellable hydrogel comprising a crosslinked mixture of a biocompatible polymer and a protein with a solution comprising at least one of diazolidinyl urea and iodopropynyl butylcarbamate.

14. The method of claim 13, wherein the solution further comprises at least one of an acid, a base, or a buffer sufficient to adjust the pH of the solution to a range of about 3.0 to about 9.0.

15. A method for delivering an agent to a wound, the method comprising applying, to a wound, a medical article comprising
a hydrophilic water-swellable hydrogel comprising a crosslinked mixture of a biocompatible polymer and a protein from a source selected from a vegetal source or a marine source, and
an agent.

16. The method of claim 15, wherein the agent is transportably present in the hydrogel.

17. The method of claim 15, wherein the agent comprises a therapeutically effective amount of a physiologically active compound to be delivered to the patient.

18. The method of claim 15, wherein the agent comprises a preservative.

19. The method of claim 15, wherein the agent comprises at least one of diazolidinyl urea and iodopropynyl butylcarbamate.

20. The method of claim 15, wherein the agent comprises lidocaine and pharmaceutically acceptable variants thereof.

21. The method of claim 15, wherein the protein comprises a soy protein.

22. The method of claim 15, wherein the hydrogel has been loaded with a solution having a pH value between about 3.0 and about 9.0.

23. A method for delivering an agent to a patient, the method comprising applying, to at least one region of a patient, a medical article comprising
a hydrophilic water-swellable hydrogel comprising a crosslinked mixture of a biocompatible polymer and a protein from a source selected from a vegetal source or a marine source, and
an agent comprising lidocaine and pharmaceutically acceptable variants thereof.

24. The method of claim 23, wherein the at least one region comprises epidermis.

25. The method of claim 23, wherein the epidermis is physically intact.
